(19) United States
(12) Patent Application Publication
(10) Pub. No.: US 2013/0236986 A1
(43) Pub. Date: Sep. 12, 2013

(54) METHODS OF SEPARATING NUCLEIC ACID POLYMER CONJUGATES

(71) Applicant: CERULEAN PHARMA INC., Cambridge, MA (US)

(72) Inventor: Roy I. Case, Westborough, MA (US)

(73) Assignee: CERULEAN PHARMA INC., Cambridge, MA (US)

(21) Appl. No.: 13/770,377

(22) Filed: Feb. 19, 2013

Related U.S. Application Data
(60) Provisional application No. 61/600,810, filed on Feb. 20, 2012.
(60) Provisional application No. 61/601,932, filed on Feb. 22, 2012.
(60) Provisional application No. 61/648,738, filed on May 18, 2012.

(51) Int. Cl. C08G 63/91; G01N 33/53

(52) U.S. Cl. CPC C08G 63/912; G01N 33/5308
USPC 436/501; 525/450

(57) ABSTRACT

Described herein are nucleic acid-polymer conjugates and methods of separating these conjugates from a mixture, such as a reaction mixture.

### CLAIM OF PRIORITY

This application claims priority to U.S. Ser. No. 61/600,810, filed Feb. 20, 2012; U.S. Ser. No. 61/601,932, filed Feb. 22, 2012; and U.S. Ser. No. 61/648,738, filed May 18, 2012, the contents of each of which are incorporated herein by reference.

### BACKGROUND

The delivery of RNA, such as silencing RNA (siRNA), using nucleic acid-hydrophobic polymer conjugates increases the efficacy and/or overall stability of the RNA. Separation systems are therefore needed that can efficiently separate these conjugates from a mixture for further processing, e.g., for incorporation into nanoparticles.

### SUMMARY

The disclosure provides, inter alia, nucleic acid-hydrophobic polymer conjugates, preparations of nucleic acid-polymer conjugates, and related methods, e.g., methods of separating nucleic acid-polymer conjugates, from a mixture, such as a reaction mixture. The nucleic acid-polymer conjugate can be made by reacting a nucleic acid (e.g., an activated nucleic acid) with a polymer (e.g., an activated polymer, e.g., an activated hydrophobic polymer), for example in a reaction mixture. The nucleic acid-polymer conjugates and related preparations, for example, a separated or purified preparation, are provided herein. Methods of evaluating the purity of the preparations are also disclosed herein. Methods of monitoring a reaction, e.g., the progression of the conjugation of a nucleic acid (e.g., an activated nucleic acid) with a polymer (e.g., an activated polymer, e.g., an activated hydrophobic polymer) are also provided herein.

The nucleic acid-polymer conjugates and related preparations can be further processed. In some embodiments, a nucleic acid-polymer conjugate or related preparation can be incorporated into a particle (e.g., a nanoparticle). The resulting particle can be formulated into a pharmaceutical composition or dosage form, which can be administered to a subject (e.g., a subject in need thereof), for example in the treatment of a disorder, e.g. a proliferative disorder, an inflammatory/autoimmune disorder, cardiovascular disorder, a metabolic disorder, a central nervous system disorder, or neurological deficit disorder.

Accordingly, in a first aspect, the disclosure provides a method of separating a nucleic acid-hydrophobic polymer conjugate from at least one other component, e.g., a contaminant, e.g., an unreacted starting material, of a mixture, the method comprising: contacting the mixture with a first phase, e.g., a stationary phase, comprising a hydrogen bond donor or acceptor; and applying a second phase, e.g., a mobile phase, comprising a polar solvent to selectively elute the nucleic acid-hydrophobic polymer conjugate from the stationary phase, thereby separating the nucleic acid-hydrophobic polymer conjugate from at least one other component of the mixture.

In some embodiments, the second phase, e.g., the mobile phase comprises a de-aggregating agent, e.g., an agent that disrupts hydrogen bonding of a nucleic acid molecule.

In some embodiments, the second phase, e.g., the mobile phase comprises a salt.

In some embodiments, the components comprise a contaminant, such as an unconjugated nucleic acid, unconjugated polymer, solvents, pyridinethiol, etc.

In some embodiments, the stationary phase can be a normal phase.

In some embodiments, the hydrogen bond donor or acceptor can be attached to a support, e.g., a solid support.

In some embodiments, the hydrogen bond donor or acceptor comprises hydroxyl moieties.

In some embodiments, the hydroxyl moieties comprise hydroxyalkyl moieties.

In some embodiments, the hydroxyalkyl moieties comprise dihydroxypropyl moieties.

In some embodiments, the support can have a diameter of 3 micrometers to 10 micrometers.

In some embodiments, the support comprises silica, e.g. porous silica, silica gel.

In some embodiments, the polar solvent comprises a polar aprotic solvent.

In some embodiments, the polar solvent comprises a plurality of polar aprotic solvents.

In some embodiments, the polar aprotic solvent can be selected from acetonitrile, N,N-dimethylformamide, acetone, cyclohexanone, methyl ethyl ketone, methyl tert-butyl ether, diethyl ether, dimethyl ether, methyl acetate, ethyl acetate and nitromethane.

In some embodiments, the polar solvent comprises a polar protic solvent.

In some embodiments, the polar protic solvent is selected from an alcohol and water.

In some embodiments, the alcohol is selected from a C₁₋₆ hydroxyalkyl, e.g., methanol, ethanol, n-propanol, isopropanol, n-butanol and isobutanol.

In some embodiments, the polar solvent comprises acetonitrile or N,N-dimethylformamide.

In some embodiments, the polar protic solvent is water.

In some embodiments, the second phase, e.g., the mobile phase can be applied as a gradient elution wherein the gradient elution increases in the % by volume of a first polar solvent and decreases in the % by volume of a second polar solvent, to elute the nucleic acid-hydrophobic polymer conjugate from the stationary phase.

In some embodiments, the % by volume of the first polar solvent increases, e.g., from 60% to at least 85%, from 60% to at least 90%, from 60% to at least 95%, or from 60% to at least 100%.

In some embodiments, the % by volume of the second polar solvent decreases from, e.g., 40% to 15% or less, 40% to 10% or less, 40% to 5% or less, or 40% to 0%.

In some embodiments, the first polar solvent comprises a solution of a salt, e.g., a lithium salt, and N,N-dimethylformamide.

In some embodiments, the second polar solvent comprises acetonitrile.

In some embodiments, the lithium salt can be lithium bromide.

In some embodiments, the first polar solvent comprises a 20 millimolar solution of lithium bromide in N,N-dimethylformamide.

In some embodiments, the method further comprises conditioning the first phase, e.g., the stationary phase with the second phase, e.g., the mobile phase.

In some embodiments, the conditioning occurs prior to contact of the mixture with the first phase, e.g., the stationary phase.

In some embodiments, the conditioning occurs after elution of the components of the mixture.

In some embodiments, the conditioning occurs prior to and again after elution of the components of the mixture.

In some embodiments, the salt can be an organic salt.

In some embodiments, the salt can be an inorganic salt.

In some embodiments, the salt can be an alkali metal halide, e.g., a lithium salt.

In some embodiments, the lithium salt can be lithium bromide.

In some embodiments, the nucleic acid comprises RNA.

In some embodiments, the nucleic acid can be an siRNA.

In some embodiments, the nucleic acid comprises DNA.

In some embodiments, the hydrophobic polymer comprises poly(lactic-co-glycolic acid) (PLGA).

In some embodiments, the PLGA is 50:50 PLGA having a weight average molecular weight ranging from about 6 kDa to about 20 kDa.

In some embodiments, the component comprises an unconjugated nucleic acid, an unconjugated hydrophobic polymer, a conjugation reaction side product, and a solvent.

In some embodiments, the mixture comprises a plurality of components selected from an unconjugated nucleic acid, an unconjugated hydrophobic polymer, a conjugation reaction side product, and a solvent.

In some embodiments, the component can be pyridinethiol. In some embodiments, the component can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the reactive moiety can be selected from an ether, thiol, amine, aldehyde, disulfanylpyridine, ester, e.g., activated ester, an azide, and an alkyne. In some embodiments, the quenched reactive moiety can be selected from a thiol, an amine, or a carboxylic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the component can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the component can be any of the reagents used in the conjugation reaction using “click chemistry”, e.g., copper catalyst or ruthenium catalyst.

In some embodiments, the component can be a solvent such as water, dimethylsulfoxide (DMSO), N,N-dimethylformamide (DMF), or acetonitrile.

In some embodiments, the component comprises a quenched unconjugated hydrophobic polymer.

In some embodiments, the quenched unconjugated hydrophobic polymer can be a hydrolyzed or reduced unconjugated hydrophobic polymer. For example, the hydrolyzed or reduced unconjugated hydrophobic polymer can comprise a thiol moiety, a carboxylic acid moiety, or an amine moiety.

In some embodiments, the component comprises an activated hydrophobic polymer, and a hydrolyzed or reduced activated hydrophobic polymer.

In some embodiments, the activated hydrophobic polymer can include a disulfanylpyridine moiety, an azide moiety, an alkyne moiety, a thiol moiety, an activated ester moiety, an amine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the hydrolyzed or reduced activated hydrophobic polymer can include a hydrophobic polymer comprising a thiol moiety, an amine moiety, or a carboxylic acid moiety.

In some embodiments, the component comprises a quenched reactive moiety.

In some embodiments, the quenched, e.g., hydrolyzed or reduced, reactive moiety, e.g., a reactive moiety that has been quenched or reduced in the conjugation reaction, can include a thiol moiety, an amine moiety, or a carboxylic acid moiety.

In some embodiments, the method further comprises providing a sample comprising eluted nucleic acid-hydrophobic polymer conjugate, e.g., eluant, e.g., a fraction of eluant.

In some embodiments, the sample comprises nucleic acid-hydrophobic polymer conjugate and a solvent.

In some embodiments, the ratio of nucleic acid-hydrophobic polymer conjugate to another component in the sample is higher than the ratio of nucleic acid-hydrophobic polymer conjugate to a component in the mixture.

In some embodiments, the ratio is at least 2-fold, 4-fold, 5-fold, 10-fold, or 50-fold higher.

In some embodiments, the sample is substantially free of another component.

In an embodiment, the component, if present, amounts to less than 5, 2, 1, 0.1, or 0.01% by weight, of the sample.

In some embodiments, the component is one or more of an unconjugated nucleic acid, an unconjugated hydrophobic polymer, a conjugation reaction side product, and a solvent.

In some embodiments, the component can be pyridinethiol. In some embodiments, the component can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the reactive moiety can be selected from an ether, thiol, amine, aldehyde, disulfanylpyridine, ester, e.g., activated ester, an azide, and an alkyne. In some embodiments, the quenched reactive moiety can be selected from a thiol, an amine, or a carboxylic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the component can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the component can be any of the reagents used in the conjugation reaction using “click chemistry”, e.g., copper catalyst or ruthenium catalyst.

In some embodiments, the component is a solvent such as water, dimethylsulfoxide (DMSO), N,N-dimethylformamide (DMF), or acetonitrile.

In some embodiments, the sample comprises less than 20, less than 15, less than 10, less than 5, less than 1, less than 0.1, or less than 0.01% by weight, or is substantially free, of an activated hydrophobic polymer.

In some embodiments, the activated hydrophobic polymer can be a hydrophobic polymer comprising a disulfanylpyridine moiety, an activated ester moiety, an azide moiety, an amine moiety, or a thiol moiety.

In some embodiments, the sample comprises less than 20, less than 15, less than 10, less than 5, less than 1, less than 0.1, or less than 0.01% by weight, or is substantially free, of a quenched activated hydrophobic polymer.

In some embodiments, the activated hydrophobic polymer can be hydrolyzed or reduced.

In some embodiments, the quenched activated hydrophobic polymer can include a thiol moiety, an amine moiety, or a carboxylic acid moiety.

In some embodiments, the sample comprises less than 20, less than 15, less than 10, less than 5, less than 1, less than 0.1, less than 0.01% by weight, or is substantially free of, an activated hydrophobic polymer and a quenched activated hydrophobic polymer.

In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the hydrophobic polymer can comprise PLGA.

In some embodiments, the method further comprises analyzing the sample for nucleic acid-hydrophobic polymer conjugate.

In another aspect, the disclosure features a method of analyzing a mixture comprising a nucleic acid-hydrophobic polymer conjugate, e.g., a preparation made by the methods described herein, for the presence of a component comprising: providing a mixture, e.g., a conjugation reaction mixture, comprising a nucleic acid or nucleic acid agent-hydrophobic polymer conjugate; and evaluating the mixture for the presence of one or more of an unconjugated nucleic acid, unconjugated hydrophobic polymer, or other conjugation reaction side product, thereby analyzing the mixture.

In some embodiments, the component can be pyridinethiol. In some embodiments, the component can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the reactive moiety can be selected from an ether, thiol, amine, aldehyde, disulfanylpyridine, ester, e.g., activated ester, an azide, and an alkyne. In some embodiments, the quenched reactive moiety can be selected from a thiol, an amine, or a carboxylic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the component can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the component can be any of the reagents used in the conjugation reaction using “click chemistry”, e.g., copper catalyst or ruthenium catalyst.

In some embodiments, the component can be a solvent such as water, dimethylsulfoxide (DMSO), N,N-dimethylformamide (DMF), or acetonitrile.

In some embodiments, the analyzing comprises applying spectrometric analysis.

In some embodiments, the spectrometric analysis can be selected from ultraviolet spectrometry, infrared spectrometry, proton nuclear magnetic resonance spectrometry (¹H-NMR), carbon-13 nuclear magnetic resonance spectrometry (¹³C-NMR), correlation nuclear magnetic resonance spectrometry (2-D NMR), ultraviolet-visible spectrometry (UV-Vis), and mass spectrometry (MS).

In some embodiments, the method further comprises reducing the volume of the sample, e.g., eluant, e.g., a fraction of eluant.

In some embodiments, the reducing can be performed prior to or after analyzing the fraction by spectrometry.

In some embodiments, the method further comprises collecting a plurality of fractions of eluant, optionally, reducing the volume of the collected eluant, and repeating the steps of the methods described above on the collected eluant, or optionally, the volume reduced collected eluant.

In some embodiments, the method further comprises providing a value, which value can be a qualitative value (e.g., present, absent, high, low, acceptable, non-acceptable) or a quantitative value (e.g., a numerical value, e.g., a single value or a range of values) for one or more components.

In some embodiments, the method further comprises comparing a value for a component with a preselected value, e.g., a reference value, a quality control value, or a release specification.

In some embodiments, the value obtained for a component can be compared to a preselected value, e.g., a reference value, a quality control value, or a release specification. In some embodiments, the reference value is 20, 15, 10, 5, 1, 0.1, 0.01%, and the observed, or obtained value, must be “less than”, or “less than or equal to”, the reference value, e.g., less than, e.g., 5%.

In some embodiments, the method further comprises, if the value of the component has a predetermined relationship with the preselected value, e.g., the reference value, e.g., if it is “less than”, “less than or equal to”, “equal to”, “greater than”, “greater than or equal to” or, e.g., in the case of a value that is a range, within the value, then the nucleic acid-hydrophobic polymer conjugate is selected for further processing, e.g., selected for incorporation into particles, e.g., nanoparticles.

In some embodiments, if the value of the component is, e.g., 5%, and the preselected value is, e.g., 20%, and the predetermined relationship is “less than or equal to”, then the sample, e.g., a sample of a batch comprising the nucleic acid-hydrophobic polymer conjugate, is selected for further processing, e.g., selected for incorporation into particles, e.g., nanoparticles.

In some embodiments, the first phase, e.g., the stationary phase, can be contacted with between 0.5 grams and 8 grams of the mixture per 100 grams of the stationary phase.

In some embodiments, the first phase, e.g., stationary phase can be contacted with between 5 grams and 100 grams of the mixture per 1 kilogram of the first phase, e.g., stationary phase.

In some embodiments, the mixture comprises at least 1 gram of the nucleic acid-hydrophobic polymer conjugate.

In some embodiments, the mixture comprises at least about 10 grams, at least about 100 grams, at least about 500 grams, or at least about 1 kilogram of the nucleic acid-hydrophobic polymer conjugate.

In some embodiments, the first phase, e.g., the stationary phase comprises silica of a diameter of between 3 micrometers and 10 micrometers covalently bonded to hydroxypropyl moieties; and the second phase, e.g., the mobile phase comprises acetonitrile, N,N-dimethylformamide, and lithium bromide.

In another aspect, the disclosure features a method of monitoring a conjugation reaction. In some embodiments, the conjugation reaction can be monitored for the progression of the conjugation of a nucleic acid (e.g., an activated nucleic acid) with a polymer (e.g., an activated polymer, e.g., an activated hydrophobic polymer), to form a nucleic acid-hydrophobic polymer conjugate. In some embodiments, the method can comprise: providing a reaction mixture, e.g., a conjugation reaction mixture comprising a nucleic acid (e.g., an activated nucleic acid) and a polymer (e.g., an activated polymer, e.g., an activated hydrophobic polymer), and analyzing the reaction mixture at one or more time intervals (e.g., for the presence, absence, and/or change in the amount of the nucleic acid (e.g., an activated nucleic acid), the polymer (e.g., an activated polymer, e.g., an activated hydrophobic polymer), and/or the nucleic acid-hydrophobic polymer conjugate), thereby monitoring the reaction mixture.

In some embodiments, the monitoring comprises removing an aliquot from the reaction mixture comprising a nucleic acid (e.g., an activated nucleic acid) and a polymer (e.g., an activated polymer, e.g., an activated hydrophobic polymer) at one or more time intervals; contacting the aliquot with a separation system described herein; and applying a detection method described herein (e.g., to detect the presence, absence, and/or change in the amount of the nucleic acid (e.g., an activated nucleic acid), the polymer (e.g., an activated polymer, e.g., an activated hydrophobic polymer), and/or the nucleic acid-hydrophobic polymer conjugate), thereby monitoring the reaction mixture.

In some embodiments, the separation system described herein comprises a first phase, e.g., a stationary phase, comprising a hydrogen bond donor or acceptor; and a second phase, e.g., a mobile phase, comprising a polar solvent. In some embodiments, the second phase, e.g., the mobile phase comprises a de-aggregating agent, e.g., an agent that disrupts hydrogen bonding of a nucleic acid molecule. In some embodiments, the second phase, e.g., the mobile phase comprises a salt. In some embodiments, the salt can be an organic salt. In some embodiments, the salt can be an inorganic salt. In some embodiments, the salt can be an alkali metal halide, e.g., a lithium salt. In some embodiments, the lithium salt can be lithium bromide.

In some embodiments, the nucleic acid comprises RNA. In some embodiments, the nucleic acid can be an siRNA. In some embodiments, the nucleic acid comprises DNA. In some embodiments, the nucleic acid comprises a reactive moiety. In some embodiments, the reactive moiety can be on either the 3′ or 5′ hydroxyl group of the nucleic acid. In some embodiments, the reactive moiety can be selected from an ether, thiol, amine, aldehyde, disulfanylpyridine, ester, e.g., activated ester, an azide, and an alkyne. In some embodiments, the reactive moiety can be a quenched reactive moiety, e.g., resulting from the quenching of the aliquot of the reaction mixture prior to contacting with the separation system, comprising a thiol, an amine, or a carboxylic acid.

In some embodiments, the hydrophobic polymer comprises poly(lactic-co-glycolic acid) (PLGA). In some embodiments, the PLGA is 50:50 PLGA having a weight average molecular weight ranging from about 6 kDa to about 20 kDa. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the activated hydrophobic polymer can be a quenched activated hydrophobic polymer, e.g., resulting from the quenching of the aliquot of the reaction mixture prior to contacting with the separation system, comprising a thiol moiety, an amine moiety, or a carboxylic acid moiety.

In some embodiments, the detection method can be selected from ultraviolet spectrometry, infrared spectrometry, proton nuclear magnetic resonance spectrometry (¹H-NMR), carbon-13 nuclear magnetic resonance spectrometry (¹³C-NMR), correlation nuclear magnetic resonance spectrometry (2-D NMR), ultraviolet-visible spectrometry (UV-Vis), and mass spectrometry (MS).

In some embodiments, the aliquot can be injected onto the column of a high performance liquid chromatography (HPLC) instrument, and the presence of the nucleic acid (e.g., an activated nucleic acid), the polymer (e.g., an activated polymer, e.g., an activated hydrophobic polymer), and the nucleic acid-hydrophobic polymer conjugate can be detected by ultraviolet (UV) spectrometry that can be coupled to the HPLC instrument.

In some embodiments, the UV spectrometry, e.g., UV detector, can comprise software that can produce a UV chromatogram. In some embodiments, the UV chromatogram can be evaluated for the presence of the nucleic acid (e.g., an activated nucleic acid), the polymer (e.g., an activated polymer, e.g., an activated hydrophobic polymer), and the nucleic acid-hydrophobic polymer conjugate using standard methods.

In some embodiments, the analyzing is performed at time 0 hours, 0.1 hour or less, 0.25 hour or less, 0.5 hour or less, 1 hour or less, 1.75 hours or less, 2 hours or less, 2.25 hours or less, 3 hours or less, 4.75 hours or less, 5 hours or less, 5.5 hours or less, 6 hours or less, 12 hours or less, and/or 24 hours or less.

In another aspect, the disclosure features a method of forming a particle comprising a nucleic acid-hydrophobic polymer conjugate, e.g., a preparation made by the methods described herein, the method comprising: combining the nucleic acid-hydrophobic polymer conjugate with a first element, thereby forming the particle.

In some embodiments, the particle can be a nanoparticle.

In some embodiments, the first element comprises a polymeric component.

In some embodiments, the first element comprises a hydrophobic polymer.

In some embodiments, the first element comprises a hydrophilic-hydrophobic polymer.

In some embodiments, the method further comprises combining the nucleic acid-hydrophobic polymer conjugate with a second element.

In some embodiments, the first element is a hydrophilic-hydrophobic polymer conjugate and the second element is a surfactant.

In some embodiments, the surfactant can be PVA.

In some embodiments, the nucleic acid-hydrophobic polymer conjugate and the first element are combined prior to generating a particle.

In some embodiments, the hydrophobic polymer can be a hydrophilic-hydrophobic polymer.

In some embodiments, the particle can be a nanoparticle.

In some embodiments, the nucleic acid-hydrophobic polymer conjugate, first element, and the second element, are combined prior to generating a particle.

In some embodiments, the particle can be generated by lyophilizing the nucleic acid-hydrophobic polymer conjugate, the first component, and the second component.

In some embodiments, the hydrophobic polymer can be a hydrophilic-hydrophobic polymer.

In some embodiments, the second component can be a surfactant, e.g., PVA.

In some embodiments, the particle can be a nanoparticle.

In some embodiments, the particle is generated by lyophilizing the nucleic acid-hydrophobic polymer conjugate, the first component, and the second component.

In another aspect, the disclosure features nucleic acid-hydrophobic polymer conjugates prepared by the methods described herein.

In another aspect, the disclosure features preparations of nucleic acid-hydrophobic polymer conjugates prepared by the methods described herein.

In another aspect, the disclosure features preparations of nucleic acid-hydrophobic polymer conjugates, made, e.g., by the methods described herein, comprising less than 30% by weight of another component.

In some embodiments, the preparation comprises less than 20%, less than 15%, less than 10%, less than 5, less than 1, less than 0.1, or less than 0.01% by weight of another component.

In some embodiments, the other component can be selected from one or more of an unconjugated nucleic acid, an unconjugated hydrophobic polymer, a conjugation reaction side product, and a solvent.

In some embodiments, the component can be pyridinethiol. In some embodiments, the component can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the component can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the component can be any of the reagents used in the conjugation reaction using “click chemistry”, e.g., copper catalyst or ruthenium catalyst.

In some embodiments, the component is a solvent such as water, dimethylsulfoxide (DMSO), N,N-dimethylformamide (DMF), or acetonitrile.

In some embodiments, the component can be an activated hydrophobic polymer.

In some embodiments, the activated hydrophobic polymer can have a reactive moiety that is still active such as an azide moiety, an activated carboxylic acid moiety, or a disulfanylpyridine moiety.

In some embodiments, the hydrophobic polymer can comprise PLGA.

In some embodiments, the component can be a quenched activated hydrophobic polymer.

In some embodiments, the component can be an activated hydrophobic polymer, and a quenched activated hydrophobic polymer.

In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the activated and/or quenched activated hydrophobic polymer can include PLGA.

In some embodiments, the component comprises one or more of an unconjugated nucleic acid agent, an unconjugated hydrophobic polymer, and a solvent, e.g., water, dimethylsulfoxide (DMSO), N,N-dimethylformamide (DMF), acetonitrile.

In another aspect, the disclosure features particles comprising the nucleic acid-hydrophobic polymer conjugate prepared by the methods described herein, a hydrophobic-hydrophilic polymer, and a surfactant.

In some embodiments, the particle further comprises a cationic moiety.

In some embodiments, the surfactant is PVA.

In another aspect, the disclosure features pharmaceutical preparations comprising particles as described herein.

In another aspect, the disclosure features methods of treating a subject in need thereof, the method comprising administering to the subject a therapeutically effective amount of the pharmaceutical preparation described herein.

In another aspect, the disclosure features methods of analyzing a preparation made by the methods described herein, for the presence of a component comprising: providing the preparation; and evaluating the preparation for the presence of one or more of an unconjugated nucleic acid, unconjugated hydrophobic polymer, or other conjugation reaction side product, thereby analyzing the preparation.

In some embodiments, the component can be pyridinethiol. In some embodiments, the component can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the component can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the component can be any of the reagents used in the conjugation reaction using “click chemistry”, e.g., copper catalyst or ruthenium catalyst.

In some embodiments, the component can be a solvent such as water, dimethylsulfoxide (DMSO), N,N-dimethylformamide (DMF), or acetonitrile.

In some embodiments, analyzing comprises applying spectrometric analysis.

In some embodiments, the spectrometric analysis can be selected from ultraviolet spectrometry, infrared spectrometry, proton nuclear magnetic resonance spectrometry (¹H-NMR), carbon-13 nuclear magnetic resonance spectrometry (¹³C-NMR), correlation nuclear magnetic resonance spectrometry (2-D NMR), ultraviolet-visible spectrometry (UV-Vis), and mass spectrometry (MS).

In some embodiments, the method further comprises providing a value, which value can be a qualitative value (e.g., present, absent, high, low, acceptable, non-acceptable) or a quantitative value (e.g., an numerical value, e.g., a single value or a range of values) for one or more components.

In some embodiments, the method further comprises comparing a value for a component with a preselected value, e.g., a reference value, a quality control value, or a release specification.

In some embodiments, the value obtained for a component can be compared to a preselected value, e.g., a reference value, a quality control value, or a release specification. In some embodiments, the reference value is 20, 15, 10, 5, 1, 0.1, 0.01%, and the observed, or obtained value, must be “less than”, or “less than or equal to”, the reference value, e.g., less than, e.g., 5%.

In some embodiments, the method further comprises, if the value of the component has a predetermined relationship with the preselected value, e.g., the reference value, e.g., if it is “less than”, “less than or equal to”, “equal to”, “greater than”, “greater than or equal to” or, e.g., in the case of a value that is a range, within the value, then the preparation, e.g., a batch comprising the preparation, is selected for further processing, e.g., selected for incorporation into particles, e.g., nanoparticles.

In some embodiments, if the value of the component is, e.g., 5%, and the preselected value is, e.g., 20%, and the predetermined relationship is “less than or equal to”, then the preparation, e.g., a batch comprising the preparation, is selected for further processing, e.g., selected for incorporation into particles, e.g., nanoparticles.

In another aspect, the disclosure features methods of analyzing a particle comprising a nucleic acid-hydrophobic polymer conjugate, e.g., a nucleic acid-hydrophobic polymer conjugate prepared by the methods described herein, the method comprising: providing the particle and analyzing the particle for the presence of a component, e.g., one or more of an unconjugated nucleic acid, an unconjugated hydrophobic polymer, or other conjugation reaction side product, thereby analyzing the particle.

In some embodiments, the analyzing comprises applying spectrometric analysis.

In some embodiments, the spectrometric analysis can be selected from cryo-scanning electron microscopy (Cryo-SEM) and transmission electron microscopy (TEM).

In some embodiments, the method further comprises providing a value, which value can be a qualitative value (e.g., present, absent, high, low, acceptable, non-acceptable) or a quantitative value (e.g., a numerical value, e.g., a single value or a range of values) for one or more components.

In some embodiments, the method further comprises comparing a value for a component with a preselected value, e.g., a reference value, a quality control value, or a release specification.

In some embodiments, the value obtained for a component can be compared to a preselected value, e.g., a reference value, a quality control value, or a release specification. In some embodiments, the reference value is 20, 15, 10, 5, 1, 0.1, 0.01%, and the observed, or obtained value, must be “less than”, or “less than or equal to”, the reference value, e.g., less than, e.g., 5%.

In some embodiments, the method further comprises, if the value of the component has a predetermined relationship with the preselected value, e.g., the reference value, e.g., if it is “less than”, “less than or equal to”, “equal to”, “greater than”, “greater than or equal to” or, e.g., in the case of a value that is a range, within the value, then the particle, e.g., a batch comprising the particle, is selected for further processing, e.g., selected for preparation of a pharmaceutical preparation.

In some embodiments, the method further comprises if the value of the component is, e.g., 5%, and the preselected value is, e.g., 20%, and the predetermined relationship is “less than or equal to”, then the particle, e.g., a batch comprising the particle, is selected for further processing, e.g., selected for preparation of a pharmaceutical preparation.

In some embodiments, the particle can be evaluated for a property as described herein. In one embodiment, the property is a physical property, e.g., average diameter. In another embodiment, the property is a functional property, e.g., the ability to mediate knockdown of a target gene, e.g., as measured in an assay described herein

In some embodiments, the component can be pyridinethiol. In some embodiments, the component can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the unconjugated nucleic acid, can include nucleic acids that do not have an reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the component can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the component can be any of the reagents used in the conjugation reaction using “click chemistry”, e.g., copper catalyst or ruthenium catalyst.

In some embodiments, the component can be a solvent such as water, dimethylsulfoxide (DMSO), N,N-dimethylformamide (DMF), or acetonitrile.

Unless otherwise defined, all technical and scientific terms used herein have the same meaning as commonly understood by one of ordinary skill in the art to which this disclosure belongs. Methods and materials are described herein for use in the disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the specification, including definitions, will control.

Other features and advantages of the disclosure will be apparent from the following detailed description and figures, and from the claims.

### BRIEF DESCRIPTION OF THE FIGURES

FIG. 1 is a line graph depicting the mobile phase gradient.

FIGS. 2A-C describe exemplary linkers which may be used to attach moieties described herein.

FIG. 3 is an ultraviolet (UV) chromatogram of an aliquot of a conjugation reaction mixture taken at a reaction time 24 hours. Integration of the nucleic acid-hydrophobic polymer conjugate peaks and free siRNA peak are shown.

FIG. 4 is a line graph showing the progression of the conjugation reaction and the formation of nucleic acid-hydrophobic polymer conjugate.

FIG. 5 is a UV chromatogram of the siRNA and siRNA dimer after 24 hours at room temperature, 40° C. and 50° C.

### DETAILED DESCRIPTION

Described herein are nucleic acid-hydrophobic polymer conjugates and methods of separating these conjugates from a mixture, such as a reaction mixture. The conjugate can be made by reacting a nucleic acid (e.g., an activated nucleic acid) with a hydrophobic polymer (e.g., an activated hydrophobic polymer). The hydrophobic polymer can be present in the reaction mixture in an excess amount relative to the amount of nucleic acid. The hydrophobic polymer comprises a reactive group, e.g., a reactive moiety that is a first component of a conjugation reaction. The nucleic acid comprises a reactive group, e.g. a reactive moiety that is a second component of a conjugation reaction. The first and second components react in the reaction mixture to form a covalent bond between the nucleic acid, and the hydrophobic polymer to form the nucleic acid-polymer conjugates. In addition to the desired product, i.e., the nucleic acid-polymer conjugate, the reaction mixture also comprises excess unconjugated hydrophobic polymer, unconjugated nucleic acid, and other conjugation reaction side products, such as pyridinethiol.

The nucleic acid-hydrophobic polymer conjugates can be separated or purified from the reaction mixture using a separation system as described herein. For example, the separation system can comprise a chromatography unit comprising a stationary phase, e.g., a solid support comprising diol moieties, and a mobile phase, e.g. a solvent system. The solvent system can comprise polar solvents and a salt, e.g. a lithium salt. The reaction mixture comprising the nucleic acid-hydrophobic polymer conjugate can be contacted with the stationary phase of the chromatographic unit. For example, the stationary phase may or may not be conditioned using the mobile phase prior to contact of the nucleic acid-hydrophobic polymer conjugate with the stationary phase. The polarity of the mobile phase is increased using a gradient solvent system, e.g., gradient elution, thereby eluting the unreacted polymer, the nucleic acid-hydrophobic polymer conjugate, and the unreacted nucleic acid. The nucleic acid-hydrophobic polymer conjugate can be separated such that the amount of the nucleic acid-hydrophobic polymer conjugate is increased relative to contaminants that are present in the reaction mixture, e.g. unreacted nucleic acid, unreacted polymer, and other conjugation reaction side products, e.g. pyridinethiol. The purity of the separated nucleic acid-hydrophobic polymer conjugate can be determined using standard analytical methods. Methods for evaluating preparations of nucleic acid-hydrophobic polymer conjugate are also described herein.

The nucleic acid-hydrophobic polymer conjugate and related preparations, which are separated by the methods described herein, can be further processed. For example, nucleic acid-hydrophobic polymer conjugates described herein can be incorporated into a particle (e.g., a nanoparticle). The resulting particle can be formulated into a pharmaceutical composition or dosage form, which can be administered to a subject (e.g., a subject in need thereof), for example in the treatment of a disorder as described herein.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particles, nucleic acid-polymer conjugates, and compositions are described herein. Also disclosed are dosage forms containing the nucleic acid-polymer conjugates, particles and compositions; methods of using the nucleic acid-polymer conjugates, particles and compositions (e.g., to treat a disorder); kits including the nucleic acid-polymer conjugates, particles and compositions; methods of making the nucleic acid-polymer conjugates, particles and compositions; methods of storing the nucleic acid-polymer conjugates, particles and compositions; and methods of analyzing the particles and compositions comprising the particles.

Headings, and other identifiers, e.g., (a), (b), (i) etc, are presented merely for ease of reading the specification and claims. The use of headings or other identifiers in the specification or claims does not require the steps or elements be performed in alphabetical or numerical order or the order in which they are presented.

### Definitions

The phrase “mobile phase” as used herein refers to the fluid phase that can or is used to move a sample, e.g., the reaction mixture, through or over a stationary phase in an analytical protocol such as a chromatography protocol.

The phrase “stationary phase” as used herein refers to a first phase, such as an immobile (relative to the second phase, e.g., mobile phase) phase involved in the separation process, e.g., a chromatography protocol. The first phase, e.g., stationary phase, can include a support, e.g., solid support, with a bonded phase, e.g., a phase that interacts with the mixture involved in the separation process.

The term “purified” as used herein refers to a preparation in which the relative amount of a nucleic acid-hydrophobic polymer conjugate, compared with at least one other component, is higher than in a reference preparation, e.g., a reaction mixture or other mixture, present, e.g., in the process of synthesizing the nucleic acid-hydrophobic polymer conjugate. In embodiments the nucleic acid-hydrophobic polymer conjugate is the only component present in post-separation mixture, sometimes referred to as the second mixture. In embodiments a purified preparation contains less than 20, 10, 5, 1, or 0.1%, by dry weight, of a component, e.g., contaminant, e.g., an unreacted starting material, e.g., unconjugated nucleic acid, unconjugated polymer, and other conjugation reaction side products, e.g., pyridinethiol. In embodiments a purified preparation is substantially free, by dry weight analysis, of a component, e.g., contaminant, e.g., an unreacted starting material, e.g., unconjugated nucleic acid, unconjugated polymer, and other conjugation reaction side products, e.g., pyridinethiol.

The term “separating” as used herein is defined as increasing the amount of a first component, e.g., a nucleic acid-hydrophobic polymer conjugate, relative to the amounts of at least one, and in embodiments, more than one, other component, e.g., a contaminant, in a first mixture, e.g., a reaction mixture. After separation, the amount of the first component in the post-separation mixture, sometimes referred to as the second mixture, is substantially increased relative to the amount of at least one, and in embodiments, more than one, of the other components. In embodiments the post-separation or second mixture contains less than 20, 10, 5, 1, or 0.1%, by dry weight, of a component, e.g., contaminant, e.g., an unreacted starting material, e.g., unconjugated nucleic acid, unconjugated polymer, and other conjugation reaction side products, e.g., pyridinethiol. In embodiments the post-separation or second mixture is substantially free, by dry weight analysis, of a component, e.g., contaminant, e.g., an unreacted starting material, e.g., unconjugated nucleic acid, unconjugated polymer, and other conjugation reaction side products, e.g., pyridinethiol. In embodiments the post-separation or second mixture contains increased levels of one or more solvents. In embodiments, the post-separation or second mixture may contain of one or more solvents, or increased levels of one or more solvents.

The term “ambient conditions,” as used herein, refers to surrounding conditions at about one atmosphere of pressure, 50% relative humidity and about 25° C., unless specified as otherwise.

The term “attach,” or “attached,” as used herein, with respect to the relationship of a first moiety to a second moiety, e.g., the attachment of an agent to a polymer, refers to the formation of a covalent bond between a first moiety and a second moiety. In the same context, the noun “attachment” refers to a covalent bond between the first and second moiety. For example, a nucleic acid attached to a polymer is a therapeutic agent, in this case a nucleic acid agent, covalently bonded to the polymer (e.g., a hydrophobic polymer described herein). The attachment can be a direct attachment, e.g., through a direct bond of the first moiety to the second moiety, or can be through a linker (e.g., through a covalently linked chain of one or more atoms disposed between the first and second moiety). For example, where an attachment is through a linker, a first moiety (e.g., a drug) is covalently bonded to a linker, which in turn is covalently bonded to a second moiety (e.g., a hydrophobic polymer described herein).

The term “biodegradable” includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. In certain embodiments, one or the other or both general types of biodegradation can occur during use of a polymer.

The term “biodegradation,” as used herein, encompasses both general types of biodegradation described above. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of a polymer, assembly of polymers or particle, and the mode and location of administration. For example, a greater molecular weight, a higher degree of crystallinity, and/or a greater biostability, usually lead to slower biodegradation.

The term “cationic moiety” refers to a moiety, which has a pKa 5 or greater (e.g., a Lewis base having a pKa of 5 or greater) and/or a positive charge in at least one of the following conditions: during the production of a particle described herein, when formulated into a particle described herein, or subsequent to administration of a particle described herein to a subject, for example, while circulating in the subject and/or while in the endosome. Exemplary cationic moieties include amine containing moieties (e.g., charged amine moieties such as a quaternary amine), guanidine containing moieties (e.g., a charged guanidine such as a quanadinium moiety), and heterocyclic and/or heteroaromatic moieties (e.g., charged moieties such as a pyridinium or a histidine moiety). Cationic moieties include polymeric species, such as moieties having more than one charge, e.g., contributed by repeated presence of a moiety, (e.g., a cationic PVA and/or a polyamine). Cationic moieties also include zwitterions, meaning a compound that has both a positive charge and a negative charge (e.g., an amino acid such as arginine, lysine, or histidine).

The term “cationic polymer,” for example, a polyamine, refers to a polymer (the term polymer is described herein below) that has a plurality of positive charges (i.e., at least 2) when formulated into a particle described herein. In some embodiments, the cationic polymer, for example, a polyamine, has at least 3, 4, 5, 10, 15, or 20 positive charges.

The phrase “cleavable under physiological conditions” refers to a bond having a half life of less than about 50 or 100 hours, when subjected to physiological conditions. For example, enzymatic degradation can occur over a period of less than about five years, one year, six months, three months, one month, fifteen days, five days, three days, or one day upon exposure to physiological conditions (e.g., an aqueous solution having a pH from about 4 to about 8, and a temperature from about 25° C. to about 37° C.

The term “contaminant,” as used herein, is a compound other than the nucleic acid-hydrophobic polymer conjugate. It can be an unconjugated component or starting material in the mixture, e.g., reaction mixture. A contaminant can be a product of the conjugation reaction other than the nucleic acid-hydrophobic polymer conjugate, such as unconjugated nucleic acid, unconjugated hydrophobic polymer, or conjugation reaction side products.

In some embodiments, the contaminant can be pyridinethiol. In some embodiments, the contaminant can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the contaminant can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the contaminant can be any of the reagents used in the conjugation reaction using “click chemistry”, e.g., copper catalyst or ruthenium catalyst.

In some embodiments, the contaminant is a solvent such as water, dimethylsulfoxide (DMSO), N,N-dimethylformamide (DMF), or acetonitrile.

An “effective amount” or “an amount effective” refers to an amount of the nucleic acid-hydrophobic polymer conjugate, particle, or composition which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition are outweighed by the therapeutically beneficial effects.

The term “embed” as used herein, refers to disposing a first moiety with, or within, a second moiety by the formation of a non-covalent interaction between the first moiety and a second moiety, e.g., a nucleic acid agent or a cationic moiety and a polymer. In some embodiments, when referring to a moiety embedded in a particle, that moiety (e.g., a nucleic acid agent or a cationic moiety) is associated with a polymer or other component of the particle through one or more non-covalent interactions such as van der Waals interactions, hydrophobic interactions, hydrogen bonding, dipole-dipole interactions, ionic interactions, and pi-stacking, and covalent bonds between the moieties and polymer or other components of the particle are absent. An embedded moiety may be completely or partially surrounded by the polymer or particle in which it is embedded.

The term “hydrogen bond” as used herein, refers to a form of association between an electronegative atom (also known as a hydrogen bond acceptor) and a hydrogen atom attached to a second, relatively electronegative atom (also known as a hydrogen bond donor). Suitable hydrogen bond donors and acceptors are known in the art.

The term “hydrogen bond acceptor” as used herein, refers to a moiety comprising an oxygen or nitrogen, e.g., an oxygen or a nitrogen that is sp²-hybridized, an ether oxygen, or the oxygen of a sulfoxide or N-oxide. They often include electronegative atoms having lone electron pairs, but also can include aromatic or unsaturated groups having pi electrons available to accept a proton from the hydrogen bond donor. Hydrogen bond acceptor groups include, but are not limited to, hydroxyl, halogen, carbonyl, lower alkoxy, esters, ethers, ketones, carbonates, amines, thiones, thioethers, thiol, sulfones, amides, and sulfide groups.

The term “hydrogen bond donor” as used herein, refers to a moiety comprising a hydrogen atom covalently linked to one electronegatively charged atom (such as oxygen or nitrogen) such that the hydrogen atom becomes electropositively charged and can thus be electrostatically attracted to interact with a second electronegative atom or group of atoms (in either case, the “hydrogen bond acceptor”). Hydrogen bond donor groups are known in the art. They include, without limitation, hydroxyl, amines (having at least one hydrogen), a primary or secondary imine group (as part of an amidine or guanidine) or a saturated or unsaturated heterocyclic group containing a ring nitrogen. Other, representative hydrogen bond donors include hydrogen covalently bonded to a nitrogen in an amide bond, and the 2-amino group of guanine.

The term “hydrophobic,” as used herein, describes a moiety that can be dissolved in an aqueous solution at physiological ionic strength only to the extent of less than about 0.05 mg/mL (e.g., about 0.01 mg/mL or less).

The term “hydrophilic,” as used herein, describes a moiety that has a solubility, in aqueous solution at physiological ionic strength, of at least about 0.05 mg/mL or greater.

The term “hydrophilic-hydrophobic polymer” as used herein, describes a polymer comprising a hydrophilic portion attached to a hydrophobic portion. Exemplary hydrophilic-hydrophobic polymers include block-copolymers, e.g., of hydrophilic and hydrophobic polymers.

A “hydroxy protecting group” or “hydroxyl protecting group” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxy protecting groups include, for example, acyl (e.g., acetyl), triethylsilyl (TES), t-butyldimethylsilyl (TBDMS), 2,2,2-trichloroethoxycarbonyl (Troc), and carbobenzyloxy (Cbz).

The term “intact,” as used herein to describe a nucleic acid, means that the nucleic acid retains a sufficient amount of structure required to carry out its biological function, e.g., to encode a protein or to effectively silence its target gene. E.g., a target gene is “effectively silenced” if its expression is decreased by at least 90%, 80%, 70%, 60%, 50%, 40%, 30%, 20% or at least 10% when contacted with the intact nucleic acid agent. Typically, in an intact preparation of nucleic acids, e.g., nucleic acid agents, e.g., siRNA, at least 60%, 70%, 80%, 90%, or all of the nucleic acid molecules have the same molecular weight or length of an intact nucleic acid molecule.

“Inert atmosphere,” as used herein, refers to an atmosphere composed primarily of an inert gas, which does not chemically react with the polymer-agent conjugates, particles, compositions or mixtures described herein. Examples of inert gases are nitrogen (N₂), helium, and argon.

“Linker,” as used herein, is a moiety that connects two or more moieties together (e.g., a nucleic acid agent or cationic moiety and a polymer such as a hydrophobic or hydrophilic-hydrophobic, or hydrophilic polymer). Linkers have at least two functional groups. For example, a linker having two functional groups may have a first functional group capable of reacting with a functional group on a moiety such as a nucleic acid a hydrophobic moiety such as a polymer, or a hydrophilic-hydrophobic polymer described herein, and a second functional group capable of reacting with a functional group on a second moiety such as a nucleic acid described herein.

A linker may have more than two functional groups (e.g., 3, 4, 5, 6, 7, 8, 9, 10 or more functional groups), which may be used, e.g., to link multiple agents to a hydrophobic polymer or to provide a biocleavable moiety within the linker. In some embodiments, for example, when a linker has more than two functional groups, e.g., and the linker comprises a functional group in addition to the two functional groups connecting a first moiety to a second moiety, the additional functional group (e.g., a third functional group) can be positioned in between the first and second group, and in some embodiments, can be cleaved, for example, under physiological conditions. For example, a linker may be of the form

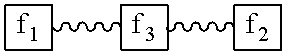

wherein f₁ is a first functional group, e.g., a functional group capable of reacting with a functional group on a moiety such as a nucleic acid a hydrophobic moiety such as a hydrophobic polymer, or a hydrophilic-hydrophobic polymer described herein; f₂ is a second functional group, e.g., a functional group capable of reacting with a functional group on a second moiety such as a nucleic acid described herein; f₃ is a biocleavable functional group, e.g., a biocleaveable bond described herein; and “ [symbol] ” represents a spacer connecting the functional groups, e.g., an alkylene (divalent alkyl) group wherein, optionally, one or more carbon atoms of the alkylene linker is replaced with one or more heteroatoms (e.g., resulting in one of the following groups: thioether, amino, ester, ether, keto, amide, silyl ether, oxime, carbamate, carbonate, disulfide, heterocyclic, or heteroaromatic). Depending on the context, linker can refer to a linker moiety before attachment to either of a first or second moiety (e.g., nucleic acid or hydrophobic polymer), after attachment to one moiety but before attachment to a second moiety, or the residue of the linker present after attachment to both the first and second moiety.

The term “lyoprotectant,” as used herein refers to a substance present in a lyophilized preparation. Typically it is present prior to the lyophilization process and persists in the resulting lyophilized preparation. Typically a lyoprotectant is added after the formation of the particles. If a concentration step is present, e.g., between formation of the particles and lyophilization, a lyoprotectant can be added before or after the concentration step. A lyoprotectant can be used to protect particles, during lyophilization, for example to reduce or prevent aggregation, particle collapse and/or other types of damage. In an embodiment the lyoprotectant is a cryoprotectant.

In an embodiment the lyoprotectant is a carbohydrate. The term “carbohydrate,” as used herein refers to and encompasses monosaccharides, disaccharides, oligosaccharides and polysaccharides.

In an embodiment, the lyoprotectant is a monosaccharide. The term “monosaccharide,” as used herein refers to a single carbohydrate unit (e.g., a simple sugar) that cannot be hydrolyzed to simpler carbohydrate units. Exemplary monosaccharide lyoprotectants include glucose, fructose, galactose, xylose, ribose and the like.

In an embodiment, the lyoprotectant is a disaccharide. The term “disaccharide,” as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide lyoprotectants include sucrose, trehalose, lactose, maltose and the like.

In an embodiment, the lyoprotectant is an oligosaccharide. The term “oligosaccharide,” as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide lyoprotectants include cyclodextrins, raffinose, melezitose, maltotriose, stachyose acarbose, and the like. An oligosaccharide can be oxidized or reduced.

In an embodiment, the lyoprotectant is a cyclic oligosaccharide. The term “cyclic oligosaccharide,” as used herein refers to a compound or a chemical moiety formed by 3 to about 15, preferably 6, 7, 8, 9, or 10 monosaccharide units that are bonded together through glycosidic linkages, for example through 1-4 linkages or 1-6 linkages, to form a cyclic structure. Exemplary cyclic oligosaccharide lyoprotectants include cyclic oligosaccharides that are discrete compounds, such as a cyclodextrin, β cyclodextrin, or γ cyclodextrin.

Other exemplary cyclic oligosaccharide lyoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety,” as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate lyoprotectants, e.g., cyclic oligosaccharide lyoprotectants, can be derivatized carbohydrates. For example, in an embodiment, the lyoprotectant is a derivatized cyclic oligosaccharide, e.g., a derivatized cyclodextrin, e.g., 2-hydroxypropyl-beta cyclodextrin, e.g., partially etherified cyclodextrins (e.g., partially etherified β cyclodextrins) disclosed in U.S. Pat. No. 6,407,079, the contents of which are incorporated herein by this reference. Another example of a derivatized cyclodextrin is β-cyclodextrin sulfobutylether sodium.

An exemplary lyoprotectant is a polysaccharide. The term “polysaccharide,” as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide lyoprotectants include glycogen, amylase, cellulose, dextran, maltodextrin and the like.

The term “derivatized carbohydrate,” refers to an entity which differs from the subject non-derivatized carbohydrate by at least one atom. For example, instead of the —OH present on a non-derivatized carbohydrate the derivatized carbohydrate can have —OX, wherein X is other than H. Derivatives may be obtained through chemical functionalization and/or substitution or through de novo synthesis—the term “derivative” implies no process-based limitation.

The term “nanoparticle” is used herein to refer to a material structure whose size in at least any one dimension (e.g., x, y, and z Cartesian dimensions) is less than about 1 micrometer (micron), e.g., less than about 500 nm or less than about 200 nm or less than about 100 nm, and greater than about 5 nm. In embodiments the size is less than about 70 nm but greater than about 20 nm. A nanoparticle can have a variety of geometrical shapes, e.g., spherical, ellipsoidal, etc. The term “nanoparticles” is used as the plural of the term “nanoparticle.”

The term “normal phase”, as used herein refers to a method or a process in which the second phase, e.g., mobile phase, is less polar than the first phase, e.g., stationary phase. In normal phase chromatography, hydrophobic analytes that exhibit more affinity for the mobile phase than for the first phase, e.g., stationary phase, elute more quickly than do hydrophilic compounds.

The term “nucleic acid” or “nucleic acid agent” refers to any synthetic or naturally occurring therapeutic agent including two or more nucleotide residues. In an embodiment the nucleic acid is an RNA, a DNA or a mixed polymer of RNA and DNA. In an embodiment an RNA is an mRNA or a siRNA. In an embodiment a DNA is a cDNA or genomic DNA. In an embodiment the nucleic acid is single stranded and in another embodiment it comprises two strands. In an embodiment the nucleic acid can have a duplexed region, comprised of strands from one or two molecules. In an embodiment the nucleic acid is an agent that inhibits gene expression, e.g., an agent that promotes RNAi. In some embodiments, the nucleic acid is siRNA, shRNA, an antisense oligonucleotide, or a microRNA (miRNA). In an embodiment the nucleic acid is an antagomir or an aptamer.

A nucleic acid can encode a peptide or protein, e.g., a therapeutic peptide or protein. The nucleic acid can be, by way of an example, an RNA, e., an mRNA, or a DNA, e.g., a nucleic acid that encodes a therapeutic protein. Exemplary therapeutic proteins include a tumor suppressor, an antigen, a cytotoxin, a cytostatin, a pro-drug activator an apoptotic protein and a protein having an anti-angiogenic activity. The nucleic acids described herein can also include one or more control regions. Exemplary control regions include, for example, an origin of replication, a promoter (e.g., a CMV promoter, or an inducible promoter), a polyadenylation signal, a Kozak sequence, an enhancer, a localization signal sequence, an internal ribosome entry sites (IRES), and a splicing signal.

As used herein, “particle polydispersity index (PDI)” or “particle polydispersity” refers to the width of the particle size distribution. Particle PDI can be calculated from the equation PDI=2a₂/a₁² where a₁ is the 1^{st} Cumulant or moment used to calculate the intensity weighted Z average mean size and a₂ is the 2^{nd} moment used to calculate a parameter defined as the polydispersity index (PdI). A particle PDI of 1 is the theoretical maximum and would be a completely flat size distribution plot. Compositions of particles described herein may have particle PDIs of less than 0.5, less than 0.4, less than 0.3, less than 0.2, or less than 0.1.

“Pharmaceutically acceptable carrier or adjuvant,” as used herein, refers to a carrier or adjuvant that may be administered to a patient, together with a nucleic acid-hydrophobic polymer conjugate, particle or composition described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The term “polymer,” as used herein, is given its ordinary meaning as used in the art, i.e., a molecular structure featuring one or more repeat units (monomers), connected by covalent bonds. The repeat units may all be identical, or in some cases, there may be more than one type of repeat unit present within the polymer. Polymers may be natural or unnatural (synthetic) polymers. Polymers may be homopolymers or copolymers containing two or more monomers. Polymers may be linear or branched.

If more than one type of repeat unit is present within the polymer, then the polymer is to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., containing one or more regions each containing a first repeat unit (e.g., a first block), and one or more regions each containing a second repeat unit (e.g., a second block), etc. Block copolymers may have two (a diblock copolymer), three (a triblock copolymer), or more numbers of distinct blocks. In terms of sequence, copolymers may be random, block, or contain a combination of random and block sequences.

In some cases, the polymer is biologically derived, i.e., a biopolymer. Non-limiting examples of biopolymers include peptides or proteins (i.e., polymers of various amino acids), or nucleic acids such as DNA or RNA.

As used herein, “polymer polydispersity index (PDI)” or “polymer polydispersity” refers to the distribution of molecular mass in a given polymer sample. The polymer PDI calculated is the weight average molecular weight divided by the number average molecular weight. It indicates the distribution of individual molecular masses in a batch of polymers. The polymer PDI has a value typically greater than 1, but as the polymer chains approach uniform chain length, the PDI approaches unity (1).

As used herein, the term “prevent” or “preventing” as used in the context of the administration of an agent to a subject, refers to subjecting the subject to a regimen, e.g., the administration of a polymer-agent conjugate, particle or composition, such that the onset of at least one symptom of the disorder is delayed as compared to what would be seen in the absence of the regimen.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a nucleic acid-hydrophobic polymer conjugate, particle or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents). Exemplary acyl groups include acetyl (CH₃C(O)—), benzoyl (C₆H₅C(O)—), and acetylamino acids (e.g., acetylglycine, CH₃C(O)NHCH₂C(O)—.

The term “alkoxy” refers to an alkyl group, as defined below, having an oxygen radical attached thereto. Representative alkoxy groups include methoxy, ethoxy, propyloxy, tert-butoxy and the like.

The term “carboxy” refers to a —C(O)OH or salt thereof.

The term “hydroxy” and “hydroxyl” are used interchangeably and refer to —OH.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

### Methods of Separating Nucleic Acid-Polymer Conjugates

Methods for separating a nucleic acid-hydrophobic polymer conjugate are described herein. Methods described herein comprise a separation system that comprises a chromatography unit, a first phase, e.g., stationary phase, and a second phase, e.g., a mobile phase. The method comprises the steps of: (i) contacting the reaction mixture comprising the nucleic acid-hydrophobic polymer conjugate with a first phase, e.g., stationary phase; (ii) separating the individual components of the reaction mixture, on the basis of polarity, by passing a second phase, e.g., a mobile phase, e.g., using a ternary gradient, comprising a first polar solvent, a second polar solvent and a salt, and a polar protic solvent through the stationary phase; and (iii) eluting each component, e.g., the unconjugated hydrophobic polymer, the nucleic acid-hydrophobic polymer conjugate, and the unconjugated nucleic acid, of the reaction mixture thereby separating the nucleic acid-hydrophobic polymer conjugate from the reaction mixture. The first phase, e.g., stationary phase, can be conditioned prior to contact with the reaction mixture using a polar solvent or a mixture thereof. The separated nucleic acid-hydrophobic polymer conjugate is detected using a detection method as described herein, and analyzed for purity using the analytical methods described herein.

### Chromatography Unit

In some embodiments, the chromatography unit is a high performance liquid chromatography (HPLC) system equipped with a column, which is packed with a stationary phase. In some embodiments, the chromatography unit is a normal phase liquid chromatography (NPLC) unit. In some embodiments, the column is a Shodex Asahi-pak column which contains a polyvinyl alcohol (PVA) packing. In some embodiments, the column is a diol normal phase column. In some embodiments, the column is an YMC-Pack Diol column. In some embodiments, the column is an Agilent Technologies LiChrospher® 100 Diol column. In some embodiments, the column is an Analtech Normal Phase Diol column. In some embodiments, the column is a GL Sciences Inertsil® diol column.

### Sample Preparation

In embodiments the conjugation reaction is carried out in a 95% dimethyl sulfoxide (DMSO), 5% tris-EDTA (TE) buffer with the activated hydrophobic polymer, e.g. PLGA-disulfanylpyridine, dissolved in DMSO and the activated nucleic acid dissolved in the buffer solution. In addition to the desired nucleic acid-hydrophobic polymer conjugate, there are other components present in the reaction mixture. For example, other components present in the conjugation reaction mixture include, but are not limited to, unconjugated nucleic acid, unconjugated hydrophobic polymer, and conjugation reaction side products (e.g., pyridinethiol leaving group). The reaction mixture can be loaded onto the column without further preparation. In some embodiments, the reaction mixture can be loaded onto the column at a concentration of up to about 1 mg/mL, up to about 2 mg/mL, up to about 3 mg/mL, up to about 4 mg/mL, up to about 5 mg/mL, up to about 10 mg/mL, up to about 15 mg/mL, up to about 20 mg/mL, up to about 25 mg/mL, up to about 30 mg/mL, up to about 35 mg/mL, or up to about 40 mg/mL of nucleic acid. In some embodiments, the conjugation reaction mixture can be further prepared prior to contact with the first phase, e.g., stationary phase. For example, the volume of the conjugation reaction mixture can be reduced using an evaporator such as a centrifugal evaporator. Examples of centrifugal evaporators can include Genevac® evaporation and concentration systems. In some embodiments, the mixture, e.g., reaction mixture, can be processed using tangential-flow filtration (TFF) before being contacted with the stationary phase.

Other preparative methods prior to contacting the nucleic acid-hydrophobic polymer conjugate with the first phase, e.g., stationary phase, can include “salting out,” which is also referred to in the art of organic chemistry as “antisolvent crystallization,” “precipitation crystallization,” “drowning out,” or “crowding out.” In this preparative method, the nucleic acid-hydrophobic polymer conjugate can be first separated from other components of the conjugation reaction mixture using a salting out method whereby a solvent is added that can facilitate the precipitation of one of the components of the conjugation reaction mixture prior to contact with the first phase, e.g., stationary phase.

### Stationary Phase

In embodiments the first phase, e.g., stationary phase used in the methods described herein comprises a support, e.g., solid support, and a bonded phase. In some embodiments, the bonded phase comprises hydrogen bond donors and/or hydrogen bond acceptors, which interact with the mixture involved in the separation process. The hydrogen bond donors and/or hydrogen bond acceptors are immobilized onto the support, e.g., solid support, by, for example, chemically binding or by insolubilizing via cross-linking. In some embodiments, the support, e.g., solid support can be macroporous, e.g. crosslinked polystyrene, polyacrylamide, polyacrylate, alumina, kieselgur (diatomaceous), quartz, kaolin, magnesium oxide, titanium dioxide or silica gel. In some embodiments, the support, e.g., solid support is silica, e.g., Silicagel. In some embodiments, the support, e.g., solid support has a diameter of at least about 1 to 150 μm, e.g., about 35 to 75 μm, or about 75 to 150 μm, about 3 to 75 μm, or about 3 to 10 μm.

In some embodiments, the support, e.g., solid support can comprise a diol phase, a glycerol phase, an amino phase, a cyano phase, a trimethylsilyl phase, a dimethylsilyl phase, a propyl phase, a butyl phase, a pentyl phase, a hexyl phase, a phenyl phase, a halogenated phase and a nitro phase. In some embodiments, the support, e.g., solid support can comprise diol moieties. For example, the diol moieties can be hydroxyalkyl moieties. In some embodiments, the hydroxyalkyl moieties can be dihydroxypropyl moieties. In some embodiments, the first phase, e.g., stationary phase is a silica gel based stationary phase bonded to diol, e.g. dihydroxypropyl, moieties.

In some embodiments, the length of the column is about 50 mm to about 250 mm. In some embodiments, the length of the column is about 50 mm. In some embodiments, the column is a preparative column, e.g. up to 1.5 meters in length. In some embodiments, the width of the column is about 1 mm, about 5 mm, about 50 mm, about 100 mm, or about 1 meter in length.

In some embodiments, the separation is performed on a kilogram scale, such as a process scale, e.g. about 1 kilogram, about 2 kilograms, about 3 kilograms, about 4 kilograms, about 5 kilograms, about 10 kilograms, using bulk silica gel. For example, the conjugation reaction mixture is contacted with the bulk silica gel, e.g., adsorbed onto the bulk silica gel. The mobile phase comprising polar aprotic solvents can then be passed over the bulk silica gel to elute the components of the conjugation reaction mixture. In some embodiments, acetonitrile can be first passed over the bulk silica gel, to elute the unconjugated polymer. Once the unconjugated polymer has eluted from the bulk silica gel, a solution of LiBr in DMF can be introduced to elute the nucleic acid-hydrophobic polymer conjugate from the bulk silica gel. Once the nucleic acid-hydrophobic polymer conjugate has eluted from the bulk silica gel, water can then be introduced to elute the unconjugated RNA. It will be understood by one of skill in the art that there are many variables to consider when purifying the nucleic acid-hydrophobic polymer conjugate on a kilogram scale, e.g., the ratio of mixture to silica gel, the solvent ratios, pore size of silica gel, etc., but these parameters are well within the realm of routine experimentation for one of skill in the art.

### Mobile Phase

In embodiments the second phase, e.g., a mobile phase used in the methods described herein comprises a first polar solvent, a second polar solvent and a salt. A polar protic solvent can be used to elute more polar components from the stationary phase. The second phase, e.g., a mobile phase can be applied to the first phase, e.g., stationary phase using a gradient elution, e.g., a ternary gradient. For example, the second phase, e.g., a mobile phase can include an increasing amount of a first polar solvent comprising a salt, and a decreasing amount of second polar solvent. A polar protic solvent can be introduced at the point at which the amount of the second polar solvent has reached a volume of about 0%.

In some embodiments, the polar solvents can include, but are not limited to acetonitrile, N,N-dimethylformamide, acetone, cyclohexanone, methyl ethyl ketone, methyl tert-butyl ether, diethyl ether, dimethyl ether, methyl acetate, ethyl acetate and nitromethane.

In some embodiments, the polar protic solvents can include, but are not limited to water, methanol, ethanol, n-propanol, isopropanol, n-butanol and isobutanol.

In some embodiments, the amount of a first polar solvent can be at least about 50% by volume, about 55% by volume, about 60% by volume, about 65% by volume, about 70% by volume, about 75% by volume, about 80% by volume, about 85% by volume, about 90% by volume, about 95% by volume, about 100% by volume.

In some embodiments, the amount of a second polar solvent can be about 50% by volume, about 45% by volume, about 40% by volume, about 35% by volume, about 30% by volume, about 25% by volume, about 15% by volume, about 10% by volume, about 5% by volume, or about 0% by volume.

In an embodiment, a polar protic solvent is introduced when the % by volume of the second polar solvent has reached 0%, i.e., the second polar solvent is not present when the aprotic solvent is introduced. In some embodiments, the amount of the polar protic solvent can be about 5% by volume, about 10% by volume, about 15% by volume, or about 20% by volume.

In some embodiments, the second phase, e.g., a mobile phase comprises a gradient elution. For example, the gradient elution can be a ternary gradient elution having a first polar solvent, e.g., a solution of a lithium salt, e.g., lithium bromide in N,N-dimethylformamide. In some embodiments, the first polar solvent can be titered from at least about 50% to about 90%, or from at least about 60% to about 100%.

In some embodiments, the solution is up to about 5 mM to about 50 mM, up to about 10 mM to about 40 mM, up to about 15 mM to about 30 mM of lithium bromide in N,N-dimethylformamide. In an embodiment, lithium bromide can be present at a concentration of 20 mM in N,N-dimethylformamide. In some embodiments, the second polar solvent, e.g., acetonitrile, can be titered from about 40% to about 0%.

In some embodiments, the polar protic solvent can be introduced into the mobile phase once the nucleic acid-hydrophobic polymer conjugate has eluted from the column. In some embodiments, the polar protic solvent can be introduced into the mobile phase when the amount of the second polar solvent, e.g., acetonitrile, has reached about 0%.

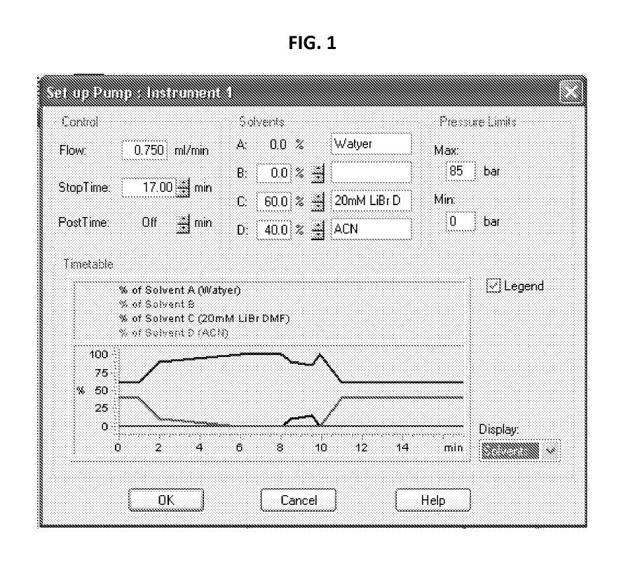

In some embodiments, the gradient elution can be performed in a linear manner. In some embodiments, the gradient elution can be performed in a stepwise manner. For example, the gradient elution can be multiphasic, e.g., biphasic, triphasic, etc. In some embodiments, the linear gradient comprises several rates of change as shown in FIG. 1. See also, e.g., Schellinger et al. Journal of Chromatography A, 1109: 253-266, 2006.

In some embodiments, the second phase, e.g., a mobile phase is passed at a flow rate of about 0.25 mL per minute, about 0.5 mL per minute, about 0.75 mL per minute, about 1 mL per minute to about 20 mL per minute. In some embodiments, the mobile phase is passed at a flow rate of about 0.75 mL per minute.

In some embodiments, the second phase, e.g., a mobile phase comprises a gradient elution as outlined in Table 1 below.

TABLE 1

| Time | % 20 mM LiBr | % | |
|---|---|---|---|
| (minutes) | in DMF | Acetonitrile | % water |
| 0.00 | 60 | 40 | 0 |
| 1.00 | 60 | 40 | 0 |
| 2.00 | 90 | 10 | 0 |
| 6.00 | 100 | 0 | 0 |
| 8.00 | 100 | 0 | 0 |
| 8.50 | 90 | 0 | 10 |
| 9.50 | 85 | 0 | 15 |
| 9.90 | 100 | 0 | 0 |
| 11.00 | 60 | 40 | 0 |

### Salts

In embodiments separation methods described herein also include the use of a salt, e.g. an organic or inorganic salt. Without wishing to be bound by theory, the addition of a salt to the second phase, e.g., a mobile phase can facilitate the break up of aggregates, e.g., hydrogen-bonded complexes, of the unreacted nucleic acid, which can cause the unreacted nucleic acid to co-elute with the nucleic acid-hydrophobic polymer conjugate. In some embodiments, the salt is an inorganic salt. For example, salts useful in the separation methods described herein can include the use of an alkali metal halide salt, which can include, but are not limited to, lithium bromide, lithium chloride, lithium fluoride, lithium iodide. In an embodiment the lithium bromide is present in a polar solvent. In some embodiments, the lithium bromide salt can be present in N,N-dimethylformamide. In some embodiments, the lithium bromide solution is from about 5 mM to about 50 mM, from about 10 mM to about 40 mM, from about 15 mM to about 30 mM in the polar solvent. In an embodiment lithium bromide is present at a concentration of 20 mM in N,N-dimethylformamide.

### Temperature

The column can be maintained at a constant temperature throughout the separation, e.g., using a commercial column heater. In some embodiments, the column can be maintained at a temperature from about 18° C. to about 70° C., e.g., about 18° C., 20° C., 22° C., 25° C., 30° C., 37° C., 40° C., 45° C., 50° C., 55° C., 60° C., 65° C., or 70° C. In some embodiments, the column is at ambient temperature.

### Detection Methods

In embodiments separation methods described herein include a method of detecting the eluant using standard detection methods. Any number of commercially available detectors, including condensation nucleation light scattering detectors (CNLSDs), charged aerosol detectors (CAD), and ultraviolet detectors (UV), may be used in the methods described herein.

In some embodiments, the eluant is detected by ultraviolet (UV) spectroscopy. In some embodiments, the eluant is detected by ultraviolet spectroscopy at a wavelength of about 220 nm, about 225 nm, about 230 nm, about 235 nm, about 240 nm, about 245 nm, about 250 nm, about 250 nm, about 255 nm, about 260 nm, about 265 nm, about 270 nm, about 275 nm, about 280 nm, about 290 nm, or about 300 nm. In some embodiments, the eluant is monitored by UV spectroscopy at a wavelength of about 270 nm to about 290 nm, of about 275 nm to about 285 nm. In some embodiments, two or more means of detection can be utilized on the same eluant, e.g., in series or in parallel.

In a preparative method, collection of the portion of the eluant containing the nucleic acid-hydrophobic polymer conjugate enriched composition can be determined by detection of the elution of the desired product, e.g., the nucleic acid-hydrophobic polymer conjugate, by UV spectroscopy.

In some embodiments, the separated nucleic acid-hydrophobic polymer conjugate are recovered after tangential-flow filtration (TFF).

In some embodiments, the second phase, e.g., the mobile phase used in the chromatography process is recycled. In some embodiments, the unconjugated nucleic acid and the unconjugated hydrophobic polymer is reused in a subsequent conjugation reaction.

### Spectrometric Analytical Methods

In embodiments separation methods described herein include the use of spectrometric analysis, to analyze the purity of the separated nucleic acid-hydrophobic polymer conjugate. Example spectrometric instruments that can be used to analyze the purity of the separated nucleic acid-hydrophobic polymer conjugates include, but are not limited to, ultraviolet (UV) spectrometry, infrared spectrometry, proton nuclear magnetic resonance spectrometry (¹H-NMR), carbon-13 nuclear magnetic resonance spectrometry (¹³C-NMR), correlation nuclear magnetic resonance spectrometry (2-D NMR), ultraviolet-visible spectrometry (UV-Vis), and mass spectrometry (MS). The purity is determined using a non-interfering wavelength, for example, using a wavelength that is above the maxima for RNA. In some embodiments, the wavelength is about 220 nm, about 225 nm, about 230 nm, about 235 nm, about 240 nm, about 245 nm, about 250 nm, about 250 nm, about 255 nm, about 260 nm, about 265 nm, about 270 nm, about 275 nm, about 280 nm, about 290 nm, or about 300 nm. In some embodiments, the eluant is monitored by UV spectroscopy at a wavelength of about 270 nm to about 290 nm, of about 275 nm to about 285 nm.

In some embodiments, the desired nucleic acid-hydrophobic polymer conjugates are recovered at a purity greater than about 60%, greater than about 70%, greater than about 75%, greater than about 80%, greater than about 85%, greater than about 90%, greater than about 95%, greater than about 98%, or greater than about 99.0%.

### Methods of Monitoring Nucleic Acid-Hydrophobic Polymer Conjugation

In embodiments the methods described herein can be used to monitor a conjugation reactions. In some embodiments, the methods described herein can be used to monitor the progression of the conjugation of a nucleic acid (e.g., an activated nucleic acid) with a polymer (e.g., an activated polymer, e.g., an activated hydrophobic polymer) to form nucleic acid-hydrophobic polymer conjugate. In some embodiments, the progression of the conjugation reaction is monitored by removing an aliquot of the conjugation reaction mixture at one or more time intervals, contacting the aliquot with the separation systems described herein, and applying a detection method as described herein (e.g., to detect the presence, absence, and/or change in the amount of the nucleic acid (e.g., an activated nucleic acid), the polymer (e.g., an activated polymer, e.g., an activated hydrophobic polymer), and/or the nucleic acid-hydrophobic polymer conjugate), thereby monitoring the conjugation reaction. In some embodiments, the aliquot can be quenched prior to contact with the separation systems described herein.

In some embodiments, an aliquot of the conjugation reaction mixture can be removed at one or more time intervals of 0 hours, 0.1 hour or less, 0.25 hour or less, 0.5 hour or less, 1 hour or less, 1.75 hours or less, 2 hours or less, 2.25 hours or less, 3 hours or less, 4.75 hours or less, 5 hours or less, 5.5 hours or less, 6 hours or less, 12 hours or less, and/or 24 hours or less, and contacted with a separation system as described herein.

In some embodiments, the aliquot is detected using UV spectrometry at a wavelength that is about 220 nm, about 225 nm, about 230 nm, about 235 nm, about 240 nm, about 245 nm, about 250 nm, about 250 nm, about 255 nm, about 260 nm, about 265 nm, about 270 nm, about 275 nm, about 280 nm, about 290 nm, or about 300 nm. In some embodiments, the reaction mixture is monitored by UV spectroscopy at a wavelength of about 270 nm to about 290 nm, of about 275 nm to about 285 nm.

In some embodiments, the aliquot can have of a volume of about 1 microliter or less, 5 microliters or less, 7 microliters or less, 10 microliters or less, or 25 microliters or less. In some embodiments, the aliquot can be removed at one or more time intervals from the conjugation reaction mixture, and injected onto the column of an HPLC instrument. In some embodiments, the HPLC instrument can be coupled to a UV spectrometer, e.g., a UV detector. In some embodiments, the UV detector comprises software to produce a UV chromatogram. In some embodiments, the peaks of the UV chromatogram can be integrated to determine the area % under the peak, or curve, and the % conjugation can be calculated. In some embodiments, the UV chromatogram comprises Agilent ChemStation™ data processing software that can integrate the area under the peak to determine the % conjugation.

### Nucleic Acid-Hydrophobic Polymer Conjugates

A nucleic acid-hydrophobic polymer conjugate described herein includes a polymer (e.g., a hydrophobic polymer) and a nucleic acid. A nucleic acid described herein may be attached to a hydrophobic polymer described herein, e.g., directly (e.g., without the presence of atoms from an intervening spacer moiety), or through a linker. A nucleic acid may be attached to a hydrophobic polymer (e.g., PLGA). A nucleic acid may be attached to a terminal end of a polymer, to both terminal ends of a polymer, or to a point along a polymer chain. In some embodiments, multiple nucleic acids, e.g., nucleic acid agents may be attached to points along a polymer chain, or multiple nucleic acids, e.g., nucleic acid agents may be attached to a terminal end of a polymer via a multifunctional linker. A nucleic acid may be attached to a polymer described herein through the 2′, 3′, or 5′ position of the nucleic acid. In embodiments where the nucleic acid is double stranded (e.g., an siRNA), the nucleic acid can be attached through the sense or antisense strand.

In some embodiment a nucleic acid-hydrophobic polymer conjugate can include a nucleic acid that forms a duplex with a nucleic acid attached to a polymer described herein. For example, a hydrophobic polymer described herein can be attached to a nucleic acid oligomer, e.g., nucleic acid agent (e.g., a single stranded DNA), which hybridizes with a nucleic acid to form a duplex. The duplex can be cleaved, releasing the nucleic acid in vivo, for example with a cellular nuclease.

### Nucleic Acids and Nucleic Acid Agents

Examples of suitable nucleic acids, e.g., nucleic acid agents include, but are not limited to polynucleotides, such as siRNA, antisense oligonucleotides, microRNAs (miRNAs), antagomirs, aptamers, genomic DNA, cDNA, mRNA, and plasmids. In some embodiments, the nucleic acid, e.g., a nucleic acid agent, can target a variety of genes of interest, such as a gene whose overexpression is associated with a disease or disorder.

The nucleic acids, e.g., nucleic acid agents delivered using a nucleic acid-polymer conjugate, particle or composition described herein can be administered alone, or in combination, (e.g., in the same or separate formulations). In one embodiment, multiple nucleic acids, e.g., nucleic acid agents, such as, siRNAs, are administered to target different sites on the same gene for treatment of a disease or disorder. In another embodiment, multiple nucleic acids, e.g., nucleic acid agents, e.g., siRNAs, are administered to target two or more different genes for treatment of a disease or disorder.

siRNA

A therapeutic nucleic acid suitable for delivery by a nucleic acid-hydrophobic polymer conjugate, particle or composition described herein can be a “short interfering RNA” or “siRNA.” As used herein, an siRNA refers to any nucleic acid molecule capable of inhibiting or down regulating gene expression or viral replication by mediating RNA interference “RNAi” or gene silencing in a sequence-specific manner. For example the siRNA can be a double-stranded nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof.

In one embodiment, the therapeutic siRNA molecule suitable for delivery with a nucleic acid-polymer conjugate, particle or composition described herein interacts with a nucleotide sequence of a target gene in a manner that causes inhibition of expression of the target gene.

siRNA comprises a double stranded structure typically containing 15-50 base pairs, e.g., 19-25, 19-23, 21-25, 21-23, or 24-29 base pairs, and having a nucleotide sequence identical or nearly identical to an expressed target gene or RNA within the cell. An siRNA may be composed of two annealed polynucleotides or a single polynucleotide that forms a hairpin structure. In one embodiment, the therapeutic siRNA is provided in the form of an expression vector, which is packaged in a nucleic acid-hydrophobic polymer conjugate, particle or composition described herein, where the vector has a coding sequence that is transcribed to produce one or more transcriptional products that produce siRNA after administration to a subject.

The siRNA can be assembled from two separate oligonucleotides, where one strand is the sense strand and the other is the antisense strand, where the antisense and sense strands are self-complementary (i.e., each strand comprises nucleotide sequence that is complementary to nucleotide sequence in the other strand); such as where the antisense strand and sense strand form a duplex or double stranded structure, for example where the double stranded region is about 15 to about 30 basepairs, e.g., about 15, 16, 17, 18, 19, 20, 21, 22, 23, 24, 25, 26, 27, 28, 29 or 30 base pairs; the antisense strand includes nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the siRNA molecule are complementary to the target nucleic acid or a portion thereof). Alternatively, the siRNA is assembled from a single oligonucleotide, where the self-complementary sense and antisense regions of the siRNA are linked by means of a nucleic acid based or non-nucleic acid-based linker(s).

In certain embodiments, at least one strand of the siRNA molecule has a 3′ overhang from about 1 to about 6 nucleotides in length, though may be from 2 to 4 nucleotides in length. Typically, the 3′ overhangs are 1-3 nucleotides in length. In some embodiments, one strand has a 3′ overhang and the other strand is blunt-ended or also has an overhang. The length of the overhangs may be the same or different for each strand. To further enhance the stability of the siRNA, the 3′ overhangs can be stabilized against degradation.

The siRNAs have significant sequence similarity to a target RNA so that the siRNAs can pair to the target RNA and result in sequence-specific degradation of the target RNA through an RNA interference mechanism. Optionally, the siRNA molecules include a 3′ hydroxyl group. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyimidine is tolerated and does not affect the efficiency of RNAi. The absence of a 2′-hydroxyl significantly enhances the nuclease resistance of the overhang in tissue culture medium and may be beneficial in vivo.

The siRNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siRNA can be a circular single-stranded polynucleotide having two or more loop structures and a stem comprising self-complementary sense and antisense regions, where the antisense region includes nucleotide sequence that is complementary to nucleotide sequence in a target nucleic acid molecule or a portion thereof and the sense region having nucleotide sequence corresponding to the target nucleic acid sequence or a portion thereof, and where the circular polynucleotide can be processed either in vivo or in vitro to generate an active siRNA molecule capable of mediating RNAi.

The siRNA can also include a single stranded polynucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), where the single stranded polynucleotide can further include a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siRNA molecule of the disclosure comprises separate sense and antisense sequences or regions, where the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions.

The siRNA need only be sufficiently similar to natural RNA that it has the ability to mediate RNAi. Thus, an siRNA can tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. In some embodiments, the agent comprises a strand that has at least about 70%, e.g., at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, such a sequence of at least 15 nucleotides, at least about 17 nucleotide, or at least about 18 or 19 to about 21-23 or 24-29 nucleotides in length. Alternatively worded, in an siRNA of about 19-25 nucleotides in length, siRNAs having no greater than about 4 mismatches are generally tolerated, as are siRNAs having no greater than 3 mismatches, 2 mismatches, and or 1 mismatch.

Mismatches in the center of the siRNA duplex are less tolerated, and may essentially abolish cleavage of the target RNA. In contrast, the 3′ nucleotides of the siRNA (e.g., the 3′ nucleotides of the siRNA antisense strand) typically do not contribute significantly to specificity of the target recognition. In particular, 3′ residues of the siRNA sequence which are complementary to the target RNA (e.g., the guide sequence) generally are not as critical for target RNA cleavage.

An siRNA suitable for delivery by a nucleic acid-hydrophobic polymer conjugate, particle or composition described herein may be defined functionally as including a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41(% G+C) (600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., et al., 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel, et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

As used herein, siRNA molecules need not be limited to those molecules containing only RNA, but may further encompass chemically-modified nucleotides and non-nucleotides. In certain embodiments, a therapeutic siRNA lacks 2′-hydroxy (2′-OH) containing nucleotides. In certain embodiments, a therapeutic siRNA does not require the presence of nucleotides having a 2′-hydroxy group for mediating RNAi and as such, an siRNA will not include any ribonucleotides (e.g., nucleotides having a 2′-OH group). Such siRNA molecules that do not require the presence of ribonucleotides to support RNAi can however have an attached linker or linkers or other attached or associated groups, moieties, or chains containing one or more nucleotides with 2′-OH groups. Optionally, an siRNA molecule can include ribonucleotides at about 5, 10, 20, 30, 40, or 50% of the nucleotide positions.

Other useful therapeutic siRNA oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No. 5,489,677, and the amide backbones disclosed in U.S. Pat. No. 5,602,240.

Substituted sugar moieties also can be included in modified oligonucleotides. Therapeutic antisense oligonucleotides for delivery by a nucleic acid-hydrophobic polymer conjugate, particle or composition described herein can include one or more of the following at the 2′ position: OH; F; O—, S—, or N-alkyl; O—, S—, or N-alkenyl; O—, S—, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃]₂, where n and m are from 1 to about 10. In addition, oligonucleotides can include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g., 2′-methoxyethoxy (2′-OCH₂CH₂OCH₃), a dimethylaminooxyethoxy group (2′-O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

An siRNA formulated with a nucleic acid-hydrophobic polymer conjugate, particle or composition described herein may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose). Suitable modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

A therapeutic siRNA for incorporation into a nucleic acid-hydrophobic polymer conjugate, particle or composition described herein may be chemically synthesized, or derived from a longer double-stranded RNA or a hairpin RNA. The siRNA can be produced enzymatically or by partial/total organic synthesis, and any modified ribonucleotide can be introduced by in vitro enzymatic or organic synthesis. A single-stranded species comprised at least in part of RNA may function as an siRNA antisense strand or may be expressed from a plasmid vector.

By “RNA interference” or “RNAi” is meant a process of inhibiting or down regulating gene expression in a cell as is generally known in the art and which is mediated by short interfering nucleic acid molecules. In addition, as used herein, the term RNAi is meant to be equivalent to other terms used to describe sequence specific RNA interference, such as post transcriptional gene silencing, translational inhibition, transcriptional inhibition, or epigenetics. For example, therapeutic siRNA molecules suitable for delivery by a nucleic acid-hydrophobic polymer conjugate, particle or composition described herein can epigenetically silence genes at both the post-transcriptional level and/or the pre-transcriptional level. In a non-limiting example, epigenetic modulation of gene expression by siRNA molecules of the disclosure can result from siRNA mediated modification of chromatin structure or methylation patterns to alter gene expression. In another non-limiting example, modulation of gene expression by an siRNA molecule can result from siRNA mediated cleavage of RNA (either coding or non-coding RNA) via RISC, or alternately, translational inhibition as is known in the art. In another embodiment, modulation of gene expression by siRNA molecules of the disclosure can result from transcriptional inhibition. RNAi also includes translational repression by microRNAs or siRNAs acting like microRNAs. RNAi can be initiated by introduction of small interfering RNAs (siRNAs) or production of siRNAs intracellularly (e.g., from a plasmid or transgene), to silence the expression of one or more target genes. Alternatively, RNAi occurs in cells naturally to remove foreign RNAs (e.g., viral RNAs). Natural RNAi proceeds via dicer-directed fragmentation of precursor dsRNA which direct the degradation mechanism to other cognate RNA sequences.

As used herein, the term siRNA is meant to be equivalent to other terms used to describe nucleic acid molecules that are capable of mediating sequence specific RNAi, and includes, for example, short interfering RNA (siRNA), double-stranded RNA (dsRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

miRNAs

In one embodiment, a therapeutic nucleic acid suitable for delivery by a nucleic acid-hydrophobic polymer conjugate, particle or composition described herein is a microRNA (miRNA). By “microRNA” or “miRNA” is meant a small double stranded RNA that regulates the expression of target messenger RNAs either by mRNA cleavage, translational repression/inhibition or heterochromatic silencing (see for example Ambros, 2004, Nature, 431, 350-355; Bartel, 2004, Cell, 116, 281-297; Cullen, 2004, Virus Research., 102, 3-9; He et al., 2004, Nat. Rev. Genet., 5, 522-531; and Ying et al., 2004, Gene, 342, 25-28). MicroRNAs (miRNAs) are small noncoding polynucleotides, about 22 nucleotides long, which direct destruction or translational repression of their mRNA targets.

In one embodiment, the therapeutic microRNA, has partial complementarity (i.e., less than 100% complementarity) between the sense strand or sense region and the antisense strand or antisense region of the miRNA molecule, or between the antisense strand or antisense region of the miRNA and a corresponding target nucleic acid molecule. For example, partial complementarity can include various mismatches or non-base paired nucleotides (e.g., 1, 2, 3, 4, 5 or more mismatches or non-based paired nucleotides, such as nucleotide bulges) within the double stranded nucleic acid molecule, structure which can result in bulges, loops, or overhangs that result between the sense strand or sense region and the antisense strand or antisense region of the miRNA or between the antisense strand or antisense region of the miRNA and a corresponding target nucleic acid molecule. Agents that act via the microRNA translational repression pathway contain at least one bulge and/or mismatch in the duplex formed with the target. In certain embodiments, a GU or UG base pair in a duplex formed by a guide strand and a target transcript is not considered a mismatch for purposes of determining whether an RNAi agent is targeted to a transcript.

In one embodiment, a therapeutic nucleic acid suitable for delivery by a nucleic acid-hydrophobic polymer conjugate, particle or composition described herein is an antagomir, which is a chemically modified oligonucleotide capable of inhibition of complementary miRNA, e.g., by promoting their degradation (see, e.g., Krutzfeldt et al., Nature, 438:685-689, 2005).

Antisense Oligonucleotides

Therapeutic “antisense oligonucleotides” are suitable for delivery via a nucleic acid-hydrophobic polymer conjugate, particle or composition described herein. The term “oligonucleotide” refers to an oligomer or polymer of ribonucleic acid (RNA) or deoxyribonucleic acid (DNA) or analogs thereof. This term includes oligonucleotides composed of naturally occurring nucleobases, sugars and covalent internucleoside (backbone) linkages, as well as oligonucleotides having non-naturally occurring portions which function similarly. Such modified or substituted oligonucleotides are often preferred over native forms because of desirable properties such as, for example, enhanced cellular uptake, enhanced affinity for a nucleic acid target, and increased stability in the presence of nucleases.

A therapeutic antisense oligonucleotide is typically from about 10 to about 50 nucleotides in length (e.g., 12 to 40, 14 to 30, or 15 to 25 nucleotides in length). Antisense oligonucleotides that are 15 to 23 nucleotides in length are particularly useful. However, an antisense oligonucleotide containing even fewer than 10 nucleotides (for example, a portion of one of the preferred antisense oligonucleotides) is understood to be included within the disclosure so long as it demonstrates the desired activity of inhibiting expression of a target gene.

An antisense oligonucleotide may consist essentially of a nucleotide sequence that specifically hybridizes with an accessible region in the target nucleic acid. Such antisense oligonucleotides, however, may contain additional flanking sequences of 5 to 10 nucleotides at either end. Flanking sequences can include, for example, additional sequences of the target nucleic acid, sequences complementary to an amplification primer, or sequences corresponding to a restriction enzyme site.

For maximal effectiveness, further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs).

While antisense oligonucleotides are a preferred form of antisense compounds, the disclosure includes other oligomeric antisense compounds, including but not limited to, oligonucleotide analogs such as those described below. As is known in the art, a nucleoside is a base-sugar combination, wherein the base portion is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric molecule. The respective ends of this linear polymeric molecule can be further joined to form a circular molecule, although linear molecules are generally preferred. Within the oligonucleotide molecule, the phosphate groups are commonly referred to as forming the internucleoside backbone of the oligonucleotide. The normal linkage or backbone of RNA and DNA is a 3′ to 5′ phosphodiester linkage.

The therapeutic antisense oligonucleotides suitable for delivery by a nucleic acid-hydrophobic polymer conjugate, particle or composition described herein include oligonucleotides containing modified backbones or non-natural internucleo side linkages. As defined herein, oligonucleotides having modified backbones include those that have a phosphorus atom in the backbone and those that do not have a phosphorus atom in the backbone. For the purposes of this specification, and as sometimes referenced in the art, modified oligonucleotides that do not have a phosphorus atom in their internucleoside backbone also can be considered to be oligonucleotides.

Modified oligonucleotide backbones can include, for example, phosphorothioates, chiral phosphorothioates, phosphorodithioates, phosphotriesters, aminoalkylphosphotriesters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkyl phosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666.

Therapeutic antisense molecules with modified oligonucleotide backbones that do not include a phosphorus atom therein can have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. References that teach the preparation of such modified backbone oligonucleotides are provided, for example, in U.S. Pat. Nos. 5,235,033 and 5,596,086.

In another embodiment, a therapeutic antisense compound is an oligonucleotide analog, in which both the sugar and the internucleoside linkage (i.e., the backbone) of the nucleotide units are replaced with novel groups, while the base units are maintained for hybridization with an appropriate nucleic acid target. One such oligonucleotide analog that has been shown to have excellent hybridization properties is referred to as a peptide nucleic acid (PNA). In PNA compounds, the sugar-backbone of an oligonucleotide is replaced with an amide containing backbone (e.g., an aminoethylglycine backbone). The nucleobases are retained and are bound directly or indirectly to aza nitrogen atoms of the amide portion of the backbone. References that teach the preparation of such modified backbone oligonucleotides are provided, for example, in Nielsen et al., Science 254:1497-1500 (1991), and in U.S. Pat. No. 5,539,082.

Other useful therapeutic antisense oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No. 5,489,677, and the amide backbones disclosed in U.S. Pat. No. 5,602,240.

Substituted sugar moieties also can be included in modified oligonucleotides. Therapeutic antisense oligonucleotides for delivery by a nucleic acid-hydrophobic polymer conjugate, particle or composition described herein can include one or more of the following at the 2′ position: OH; F; O—, S—, or N-alkyl; O—, S—, or N-alkenyl; O—, S—, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃]₂, where n and m are from 1 to about 10. In addition, oligonucleotides can include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g., 2′-methoxyethoxy (2′-OCH₂CH₂OCH₃), a dimethylaminooxyethoxy group (2′-O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

Therapeutic antisense oligonucleotides can also include nucleobase modifications or substitutions. As used herein, “unmodified” or “natural” nucleobases include the purine bases adenine (A) and guanine (G), and the pyrimidine bases thymine (T), cytosine (C), and uracil (U). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Certain nucleobase substitutions can be particularly useful for increasing the binding affinity of the antisense oligonucleotides of the disclosure. For example, 5-methylcytosine substitutions have been shown to increase nucleic acid duplex stability by 0.6 to 1.2° C. (Sanghvi et al., eds., Antisense Research and Applications, pp. 276-278, CRC Press, Boca Raton, Fla. (1993)). Other useful nucleobase substitutions include 5-substituted pyrimidines, 6-azapyrimidines and N-2, N-6 and O-6 substituted purines such as 2-aminopropyladenine, 5-propynyluracil and 5-propynylcytosine.

It is not necessary for all nucleobase positions in a given antisense oligonucleotide be uniformly modified. More than one of the aforementioned modifications can be incorporated into a single oligonucleotide or even at a single nucleoside within an oligonucleotide. The therapeutic nucleic acids suitable for delivery by a conjugate, particle or compositions described herein also include antisense oligonucleotides that are chimeric oligonucleotides. “Chimeric” antisense oligonucleotides can contain two or more chemically distinct regions, each made up of at least one monomer unit (e.g., a nucleotide in the case of an oligonucleotide). Chimeric oligonucleotides typically contain at least one region wherein the oligonucleotide is modified so as to confer, for example, increased resistance to nuclease degradation, increased cellular uptake, and/or increased affinity for the target nucleic acid. For example, a region of a chimeric oligonucleotide can serve as a substrate for an enzyme such as RNase H, which is capable of cleaving the RNA strand of an RNA:DNA duplex such as that formed between a target mRNA and an antisense oligonucleotide. Cleavage of such a duplex by RNase H, therefore, can greatly enhance the effectiveness of an antisense oligonucleotide.

The therapeutic antisense oligonucleotides can be synthesized in vitro. Antisense oligonucleotides used in accordance with this disclosure can be conveniently produced through known methods, e.g., by solid phase synthesis. Similar techniques also can be used to prepare modified oligonucleotides such as phosphorothioates or alkylated derivatives.

Antisense polynucleotides include sequences that are complementary to a genes or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

The term “hybridization,” as used herein, means hydrogen bonding, which can be Watson-Crick, Hoogsteen, or reversed Hoogsteen hydrogen bonding, between complementary nucleoside or nucleotide bases. For example, adenine and thymine, and guanine and cytosine, respectively, are complementary nucleobases (often referred to in the art simply as “bases”) that pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide in a target nucleic acid molecule, then the oligonucleotide and the target nucleic acid are considered to be complementary to each other at that position. The oligonucleotide and the target nucleic acid are complementary to each other when a sufficient number of corresponding positions in each molecule are occupied by nucleotides that can hydrogen bond with each other. Thus, “specifically hybridizable” is used to indicate a sufficient degree of complementarity or precise pairing such that stable and specific binding occurs between the oligonucleotide and the target nucleic acid.

It is understood in the art that the sequence of an antisense oligonucleotide need not be 100% complementary to that of its target nucleic acid to be specifically hybridizable. An antisense oligonucleotide is specifically hybridizable when (a) binding of the oligonucleotide to the target nucleic acid interferes with the normal function of the target nucleic acid, and (b) there is sufficient complementarity to avoid non-specific binding of the antisense oligonucleotide to non-target sequences under conditions in which specific binding is desired, i.e., under conditions in which in vitro assays are performed or under physiological conditions for in vivo assays or therapeutic uses.

Stringency conditions in vitro are dependent on temperature, time, and salt concentration (see e.g., Sambrook et al., Molecular Cloning: A Laboratory Manual, Cold Spring Harbor Laboratory Press, NY (1989)). Typically, conditions of high to moderate stringency are used for specific hybridization in vitro, such that hybridization occurs between substantially similar nucleic acids, but not between dissimilar nucleic acids. Specific hybridization conditions are hybridization in 5×SSC (0.75 M sodium chloride/0.075 M sodium citrate) for 1 hour at 40° C., followed by washing 10 times in 1×SSC at 40° C. and 5× in 1×SSC at room temperature.

In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

The specific hybridization of an antisense molecule with its target nucleic acid can interfere with the normal function of the target nucleic acid. For a target DNA nucleic acid, antisense technology can disrupt replication and transcription. For a target RNA nucleic acid, antisense technology can disrupt, for example, translocation of the RNA to the site of protein translation, translation of protein from the RNA, splicing of the RNA to yield one or more mRNA species, and catalytic activity of the RNA. The overall effect of such interference with target nucleic acid function is, in the case of a nucleic acid encoding a target gene, inhibition of the expression of target gene. In the context of the disclosure, “inhibiting expression of a target gene” means to disrupt the transcription and/or translation of the target nucleic acid sequences resulting in a reduction in the level of target polypeptide or a complete absence of target polypeptide.

An antisense oligonucleotide, e.g., an antisense strand of a siRNA may preferably be directed at specific targets within a target nucleic acid molecule. The targeting process includes the identification of a site or sites within the target nucleic acid molecule where an antisense interaction can occur such that a desired effect, e.g., inhibition of target gene expression, will result. Traditionally, preferred target sites for antisense oligonucleotides have included the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions.

Simple knowledge of the sequence and domain structure (e.g., the location of translation initiation codons, exons, or introns) of a target nucleic acid, however, is generally not sufficient to ensure that an antisense oligonucleotide directed to a specific region will effectively bind to and inhibit transcription and/or translation of the target nucleic acid. In its native state, an mRNA molecule is folded into complex secondary and tertiary structures, and sequences that are on the interior of such structures are inaccessible to antisense oligonucleotides. For maximal effectiveness, antisense oligonucleotides can be directed to regions of a target mRNA that are most accessible, i.e., regions at or near the surface of a folded mRNA molecule. Accessible regions of an mRNA molecule can be identified by methods known in the art, including the use of RiboTAG™, or mRNA Accessible Site Tagging (MAST), technology. RiboTAG™ technology is disclosed in PCT Application Number SE01/02054.

Once one or more target sites have been identified, antisense oligonucleotides can be synthesized that are sufficiently complementary to the target (i.e., that hybridize with sufficient strength and specificity to give the desired effect). The effectiveness of an antisense oligonucleotide to inhibit expression of a target nucleic acid can be evaluated by measuring levels of target mRNA or protein using, for example, Northern blotting, RT-PCR, Western blotting, ELISA, or immunohistochemical staining.

In some embodiments, it may be useful to target multiple accessible regions of a target nucleic acid. In such embodiments, multiple antisense oligonucleotides can be used that each specifically hybridize to a different accessible region. Multiple antisense oligonucleotides can be used together or sequentially. In some embodiments, it may be useful to target multiple accessible regions of multiple target nucleic acids.

Aptamers

A therapeutic nucleic acid suitable for delivery by a nucleic acid-hydrophobic polymer conjugate, particle or composition described herein can be an aptamer (also called a nucleic acid ligand or nucleic acid aptamer), which is a polynucleotide that binds specifically to a target molecule where the nucleic acid molecule has a sequence that is distinct from a sequence recognized by the target molecule in its natural setting. Alternately, an aptamer can be a nucleic acid molecule that binds to a target molecule where the target molecule does not naturally bind to a nucleic acid. The target molecule can be any molecule of interest. The target molecule can be, for example, a polypeptide, a carbohydrate, a nucleic acid molecule or a cell. The target of an aptamer is a three dimensional chemical structure that binds to the aptamer. For example, an aptamer that targets a nucleic acid (e.g., an RNA or a DNA) may include regions that bind via complementary Watson-Crick base pairing to a nucleic acid target interrupted by other structures such as hairpin loops. In another embodiment, the aptamer binds a target protein at a ligand-binding domain, thereby preventing interaction of the naturally occurring ligand with the target protein.

In one embodiment, the aptamer binds to a cell or tissue in a specific developmental stage or a specific disease state. A target is an antigen on the surface of a cell, such as a cell surface receptor, an integrin, a transmembrane protein, an ion channel or a membrane transport protein. In one embodiment, the target is a tumor-marker. A tumor-marker can be an antigen that is present in a tumor that is not present in normal tissue or an antigen that is more prevalent in a tumor than in normal tissue.

The nucleic acid that forms the nucleic acid ligand may be composed of naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In one embodiment, nucleotides or modified nucleotides of the nucleic acid ligand can be replaced with a hydrocarbon linker or a polyether linker provided that the binding affinity and selectivity of the nucleic acid ligand is not substantially reduced by the substitution (e.g., the dissociation constant of the aptamer for the target is typically not greater than about 1×10⁻⁶ M).

An aptamer may be prepared by any method, such as by Systemic Evolution of Ligands by Exponential Enrichment (SELEX). The SELEX process for obtaining nucleic acid ligands is described in U.S. Pat. No. 5,567,588, the entire teachings of which are incorporated herein by reference.

Within the particles described herein, the nucleic acids, e.g., nucleic acid agents can be attached to another moiety such as a hydrophobic polymer described above. The nucleic acids, e.g., nucleic acid agents can also be “free,” meaning not attached to another moiety. Where a particle includes a plurality of nucleic acids, e.g., nucleic acid agents, some of the nucleic acids, e.g., nucleic acid agents can be attached to another moiety and some can be free. For example, in certain embodiments, the nucleic acids, e.g., nucleic acid agents in the particle are attached to a polymer of the particle. The nucleic acids, e.g., nucleic acid agents may be attached to any polymer in the particle, e.g., a hydrophobic polymer or a polymer containing a hydrophilic and a hydrophobic portion.

In certain embodiments, a nucleic acid is “free” in the particle. The nucleic acids, e.g., nucleic acid agents may be associated with a polymer or other component of the particle through one or more non-covalent interactions such as van der Waals interactions, hydrophobic interactions, hydrogen bonding, dipole-dipole interactions, ionic interactions, and pi stacking. A nucleic acid may be present in varying amounts of a nucleic acid-hydrophobic polymer conjugate, particle or composition described herein. When present in a particle, the nucleic acid may be present in an amount, e.g., from about 0.1 to about 50% by weight of the particle (e.g., from about 1% to about 50%, from about 1 to about 30% by weight of the particle, from about 1 to about 20% by weight of the particle, from about 4 to about 25% by weight of the particle, or from about 5 to about 13%, 14%, 15%, 16%, 17%, 18%, 19% or 20% by weight of the particle).

### Hydrophobic Polymers

Nucleic acid-hydrophobic polymer conjugates disclosed herein include a hydrophobic polymer. Particles described herein may include a hydrophobic polymer. The hydrophobic polymer may be attached to a nucleic acid and/or cationic moiety to form a conjugate (e.g., a nucleic acid agent-hydrophobic polymer conjugate or cationic moiety-hydrophobic polymer conjugate). In some embodiments, the nucleic acid forms a duplex with a nucleic acid that is attached to the hydrophobic polymer.

In some embodiments, the hydrophobic polymer is not attached to another moiety. A particle can include a plurality of hydrophobic polymers, for example where some are attached to another moiety such as a nucleic acid and/or cationic moiety and some are free.

In some embodiments, the hydrophobic polymer can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the dimerized hydrophobic polymer can have a weight average molecular weight of about 2 kDa to about 140 kDa. In some embodiments, the hydrophobic polymer can comprise PLGA.

Exemplary hydrophobic polymers include the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate; cellulose acetate succinate; hydroxypropylmethylcellulose phthalate; poly(D,L-lactide); poly(D,L-lactide-co-glycolide); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers (see generally, Svenson, S (ed.)., Polymeric Drug Delivery: Volume I: Particulate Drug Carriers. 2006; ACS Symposium Series; Amiji, M. M (ed.)., Nanotechnology for Cancer Therapy. 2007; Taylor & Francis Group, LLP; Nair et al. Prog. Polym. Sci. (2007) 32: 762-798); hydrophobic peptide-based polymers and copolymers based on poly(L-amino acids) (Lavasanifar, A., et al., Advanced Drug Delivery Reviews (2002) 54:169-190); poly(ethylene-vinyl acetate) (“EVA”) copolymers; silicone rubber; polyethylene; polypropylene; polydienes (polybutadiene, polyisoprene and hydrogenated forms of these polymers); maleic anhydride copolymers of vinyl methylether and other vinyl ethers; polyamides (nylon 6,6); polyurethane; poly(ester urethanes); poly(ether urethanes); and poly(ester-urea).

Hydrophobic polymers useful in preparing the nucleic acid-hydrophobic polymer conjugates or particles described herein also include biodegradable polymers. Examples of biodegradable polymers include polylactides, polyglycolides, caprolactone-based polymers, poly(caprolactone), polydioxanone, polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), poly(vinylpyrrolidone), polyethylene glycol, polyhydroxycellulose, polysaccharides, chitin, chitosan and hyaluronic acid, and copolymers, terpolymers and mixtures thereof. Biodegradable polymers also include copolymers, including caprolactone-based polymers, polycaprolactones and copolymers that include polybutylene terephthalate.

In some embodiments, the polymer is a polyester synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, and malic acid.

A copolymer may also be used in a polymer-agent conjugate or particle described herein. In some embodiments, a polymer may be PLGA, which is a biodegradable random copolymer of lactic acid and glycolic acid. A PLGA polymer may have varying ratios of lactic acid:glycolic acid, e.g., ranging from about 0.1:99.9 to about 99.9:0.1 (e.g., from about 75:25 to about 25:75, from about 60:40 to 40:60, or about 55:45 to 45:55). In some embodiments, e.g., in PLGA, the ratio of lactic acid monomers to glycolic acid monomers is 50:50, 60:40 or 75:25.

In particular embodiments, by optimizing the ratio of lactic acid to glycolic acid monomers in the PLGA polymer of the polymer-agent conjugate or particle, parameters such as water uptake, agent release (e.g., “controlled release”) and polymer degradation kinetics may be optimized. Furthermore, tuning the ratio will also affect the hydrophobicity of the copolymer, which may in turn affect drug loading.

In certain embodiments wherein the biodegradable polymer also has a nucleic acid or other material such as a cationic moiety attached to it or a nucleic acid that forms a duplex with a nucleic acid attached to it, the biodegradation rate of such polymer may be characterized by a release rate of such materials. In such circumstances, the biodegradation rate may depend on not only the chemical identity and physical characteristics of the polymer, but also on the identity of material(s) attached thereto. Degradation of the subject compositions includes not only the cleavage of intramolecular bonds, e.g., by oxidation and/or hydrolysis, but also the disruption of intermolecular bonds, such as dissociation of host/guest complexes by competitive complex formation with foreign inclusion hosts. In some embodiments, the release can be affected by an additional component in the particle, e.g., a compound having at least one acidic moiety (e.g., free-acid PLGA).

In certain embodiments, particles comprising one or more polymers, such as a hydrophobic polymer, biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 4 and 8 having a temperature of between 25° C. and 37° C. In other embodiments, the polymer degrades in a period of between about one hour and several weeks, depending on the desired application.

When polymers are used for delivery of nucleic acid agents in vivo, it is important that the polymers themselves be nontoxic and that they degrade into non-toxic degradation products as the polymer is eroded by the body fluids. Many synthetic biodegradable polymers, however, yield oligomers and monomers upon erosion in vivo that adversely interact with the surrounding tissue (D. F. Williams, J. Mater. Sci. 1233 (1982)). To minimize the toxicity of the intact polymer carrier and its degradation products, polymers have been designed based on naturally occurring metabolites. Exemplary polymers include polyesters derived from lactic and/or glycolic acid and polyamides derived from amino acids.

A number of biodegradable polymers are known and used for controlled release of pharmaceuticals. Such polymers are described in, for example, U.S. Pat. Nos. 4,291,013; 4,347,234; 4,525,495; 4,570,629; 4,572,832; 4,587,268; 4,638,045; 4,675,381; 4,745,160; and 5,219,980; and PCT publication WO2006/014626, each of which is hereby incorporated by reference in its entirety.

A hydrophobic polymer described herein may have a variety of end groups. In some embodiments, the end group of the polymer is not further modified, e.g., when the end group is a carboxylic acid, a hydroxy group or an amino group. In some embodiments, the end group may be further modified. For example, a polymer with a hydroxyl end group may be derivatized with an acyl group to yield an acyl-capped polymer (e.g., an acetyl-capped polymer or a benzoyl capped polymer), an alkyl group to yield an alkoxy-capped polymer (e.g., a methoxy-capped polymer), or a benzyl group to yield a benzyl-capped polymer. The end group can also be further reacted with a functional group, for example to provide a linkage to another moiety such as a nucleic acid, e.g., nucleic acid agent, a cationic moiety, or an insoluble substrate. In some embodiments a particle comprises a functionalized hydrophobic polymer, e.g., a hydrophobic polymer, such as PLGA (e.g., 50:50 PLGA), functionalized with a moiety, e.g., N-(2-aminoethyl)maleimide, 2-(2-(pyridine-2-yl)disulfanyl)ethylamino, or a succinimidyl-N-methyl ester, that has not reacted with another moiety, e.g., a nucleic acid.

A hydrophobic polymer may have a weight average molecular weight ranging from about 1 kDa to about 70 kDa (e.g., from about 4 kDa to about 66 kDa, from about 2 kDa to about 12 kDa, from about 6 kDa to about 20 kDa, from about 5 kDa to about 15 kDa, from about 6 kDa to about 13 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the hydrophobic polymer is PLGA, e.g., 50:50 PLGA having a weight average molecular weight ranging from about 6 kDa to about 20 kDa.

A hydrophobic polymer described herein may have a polymer polydispersity index (PDI) of less than or equal to about 2.5 (e.g., less than or equal to about 2.2, less than or equal to about 2.0, or less than or equal to about 1.5). In some embodiments, a hydrophobic polymer described herein may have a polymer PDI of about 1.0 to about 2.5, about 1.0 to about 2.0, about 1.0 to about 1.7, or from about 1.0 to about 1.6.

A particle described herein may include varying amounts of a hydrophobic polymer, e.g., from about 10% to about 90% by weight of the particle (e.g., from about 20% to about 80%, from about 25% to about 75%, or from about 30% to about 70%).

A hydrophobic polymer described herein may be commercially available, e.g., from a commercial supplier such as BASF, Boehringer Ingelheim, Durcet Corporation, Purac America and SurModics Pharmaceuticals. A polymer described herein may also be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization.

A commercially available or synthesized polymer sample may be further purified prior to formation of a polymer-agent conjugate or incorporation into a particle or composition described herein. In some embodiments, purification may reduce the polydispersity of the polymer sample. A polymer may be purified by precipitation from solution, or precipitation onto a solid such as Celite. A polymer may also be further purified by size exclusion chromatography (SEC).

### Hydrophobic-Hydrophilic Polymers

The conjugates disclosed herein can be included as a component of a particle. The particles can include other elements, e.g., a hydrophobic-hydrophilic polymer. Described herein are polymers containing a hydrophilic portion and a hydrophobic portion, e.g., a hydrophobic-hydrophilic polymer. The hydrophobic-hydrophilic polymer may be attached to another moiety such as a nucleic acid (e.g., through the hydrophilic or hydrophobic portion) and/or a cationic moiety or a nucleic acid can form a duplex with a nucleic acid attached to the hydrophobic-hydrophilic polymer. In some embodiments, the hydrophobic-hydrophilic polymer is free (i.e., not attached to another moiety). A particle can include a plurality of hydrophobic-hydrophilic polymers, for example where some are attached to another moiety such as a nucleic acid and/or cationic moiety and some are free.

A polymer containing a hydrophilic portion and a hydrophobic portion may be a copolymer of a hydrophilic block coupled with a hydrophobic block. These copolymers may have a weight average molecular weight between about 5 kDa and about 30 kDa (e.g., from about 5 kDa to about 25 kDa, from about 10 kDa to about 22 kDa, from about 10 kDa to about 15 kDa, from about 12 kDa to about 22 kDa, from about 7 kDa to about 15 kDa, from about 15 kDa to about 19 kDa, or from about 11 kDa to about 13 kDa, e.g., about 9 kDa, about 10 kDa, about 11 kDa, about 12 kDa, about 13 kDa, about 14 kDa about 15 kDa, about 16 kDa, about 17 kDa, about 18 kDa or about 19 kDa). The polymer containing a hydrophilic portion and a hydrophobic portion may be attached to an agent.

Examples of suitable hydrophobic portions of the polymers include those described above. The hydrophobic portion of the copolymer may have a weight average molecular weight of from about 1 kDa to about 20 kDa (e.g., from about 8 kDa to about 15, kDa from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

Examples of suitable hydrophilic portions of the polymers include the following: carboxylic acids including acrylic acid, methacrylic acid, itaconic acid, and maleic acid; polyoxyethylenes or polyethylene oxide (PEG); polyacrylamides (e.g. polyhydroxylpropylmethacrylamide), and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, poly(vinylpyrrolidone), polyoxazoline, polysialic acid, starches and starch derivatives, dextran and dextran derivatives; polypeptides, such as polylysines, polyarginines, polyglutamic acids; polyhyaluronic acids, alginic acids, polylactides, polyethyleneimines, polyionenes, polyacrylic acids, and polyiminocarboxylates, gelatin, and unsaturated ethylenic mono or dicarboxylic acids. A listing of suitable hydrophilic polymers can be found in Handbook of Water-Soluble Gums and Resins, R. Davidson, McGraw-Hill (1980). The hydrophilic portion of the copolymer may have a weight average molecular weight of from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is PEG, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is PVA, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyoxazoline, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyvinylpyrrolidine, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyhydroxylpropylmethacrylamide, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polysialic acid, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa).

A polymer containing a hydrophilic portion and a hydrophobic portion may be a block copolymer, e.g., a diblock or triblock copolymer. In some embodiments, the polymer may be a diblock copolymer containing a hydrophilic block and a hydrophobic block. In some embodiments, the polymer may be a triblock copolymer containing a hydrophobic block, a hydrophilic block and another hydrophobic block. The two hydrophobic blocks may be the same hydrophobic polymer or different hydrophobic polymers. The block copolymers used herein may have varying ratios of the hydrophilic portion to the hydrophobic portion, e.g., ranging from 1:1 to 1:40 by weight (e.g., about 1:1 to about 1:10 by weight, about 1:1 to about 1:2 by weight, or about 1:3 to about 1:6 by weight).

A polymer containing a hydrophilic portion and a hydrophobic portion may have a variety of end groups. In some embodiments, the end group may be a hydroxy group or an alkoxy group (e.g., methoxy). In some embodiments, the end group of the polymer is not further modified. In some embodiments, the end group may be further modified. For example, the end group may be capped with an alkyl group, to yield an alkoxy-capped polymer (e.g., a methoxy-capped polymer), may be derivatized with a targeting agent (e.g., folate) or a dye (e.g., rhodamine), or may be reacted with a functional group.

A polymer containing a hydrophilic portion and a hydrophobic portion may include a linker between the two blocks of the copolymer. Such a linker may be an amide, ester, ether, amino, carbamate or carbonate linkage, for example.

A polymer containing a hydrophilic portion and a hydrophobic portion described herein may have a polymer polydispersity index (PDI) of less than or equal to about 2.5 (e.g., less than or equal to about 2.2, or less than or equal to about 2.0, or less than or equal to about 1.5). In some embodiments, the polymer PDI is from about 1.0 to about 2.5, e.g., from about 1.0 to about 2.0, from about 1.0 to about 1.8, from about 1.0 to about 1.7, or from about 1.0 to about 1.6.

A particle described herein may include varying amounts of a polymer containing a hydrophilic portion and a hydrophobic portion, e.g., up to about 50% by weight of the particle (e.g., from about 4 to about 50%, about 5%, about 10%, about 15%, about 20%, about 25%, about 30%, about 35%, about 40%, about 45% or about 50% by weight). For example, the percent by weight of the second polymer within the particle is from about 3% to 30%, from about 5% to 25% or from about 8% to 23%.

A polymer containing a hydrophilic portion and a hydrophobic portion described herein may be commercially available, or may be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization. A block copolymer may be prepared by synthesizing the two polymer units separately and then conjugating the two portions using established methods. For example, the blocks may be linked using a coupling agent such as EDC (1-ethyl-3-(3-dimethylaminopropyl)carbodiimide hydrochloride). Following conjugation, the two blocks may be linked via an amide, ester, ether, amino, carbamate or carbonate linkage.

A commercially available or synthesized polymer sample may be further purified prior to formation of a polymer-agent conjugate or incorporation into a particle or composition described herein. In some embodiments, purification may remove lower molecular weight polymers that may lead to unfilterable polymer samples. A polymer may be purified by precipitation from solution, or precipitation onto a solid such as Celite. A polymer may also be further purified by size exclusion chromatography (SEC).

### Cationic Moieties

The conjugates disclosed herein can be included as a composent of a particle. The particles can include other elements, e.g., a cationic moiety. Exemplary cationic moieties for use in the particles described herein include amines, including for example, primary, secondary, tertiary, and quaternary amines, and polyamines (e.g., branched and linear polyethylene imine (PEI) or derivatives thereof such as polyethyleneimine-PLGA, polyethylene imine-polyethylene glycol-N-acetylgalactosamine (PEI-PEG-GAL) or polyethylene imine-polyethylene glycol-tri-N-acetylgalactosamine (PEI-PEG-triGAL) derivatives). In some embodiments, the cationic moiety comprises a cationic lipid (e.g., 1-[2-(oleoyloxy)ethyl]-2-oleyl-3-(2-hydroxyethyl)imidazolinium chloride (DOTIM), dimethyldioctadecyl ammonium bromide, 1,2 dioleyloxypropyl-3-trimethyl ammonium bromide, DOTAP, 1,2-dimyristyloxypropyl-3-dimethyl-hydroxyethyl ammonium bromide, 1,2-dimyristoyl-sn-glycero-3-ethylphosphocholine (EDMPC), ethyl-PC, 1,2-dioleoyl-3-dimethylammonium-propane (DODAP), DC-cholesterol, and MBOP, CLinDMA, 1,2-dilinoleyloxy-3-dimethylaminopropane (DLinDMA), pCLinDMA, eCLinDMA, DMOBA, and DMLBA). In some embodiments, for example, where the cationinic moiety is a polyamine, the polyamine comprises, polyamino acids (e.g., poly(lysine), poly(histidine), and poly(arginine)) and derivatives (e.g. poly(lysine)-PLGA, imidazole modified poly(lysine)) or polyvinyl pyrrolidone (PVP). In some embodiments, for example, where the cationic moiety is a cationic polymer comprising a plurality of amines, the amines can be positioned along the polymer such that the amines are from about 4 to about 10 angstroms apart (e.g., from about 5 to about 8 or from about 6 to about 7). In some embodiments, the amines can be positioned along the polymer so as to be in register with phosphates on a nucleic acid agent.

The cationic moiety can have a pKa of 5 or greater and/or be positively charged at physiological pH.

In some embodiments, the cationic moiety includes at least one amine (e.g., a primary, secondary, tertiary or quaternary amine), or a plurality of amines, each independently a primary, secondary, tertiary or quaternary amine). In some embodiments the cationic moiety is a polymer, for example, having one or more secondary or tertiary amines, for example cationic polyvinyl alcohol (PVA) (e.g., as provided by Kuraray, such as CM-318 or C-506), chitosan, polyamine-branched and star PEG and polyethylene imine. Cationic PVA can be made, for example, by polymerizing a vinyl acetate/N-vinaylformamide co-polymer, e.g., as described in US 2002/0189774, the contents of which are incorporated herein by reference. Other examples of cationic PVA include those described in U.S. Pat. No. 6,368,456 and Fatehi (Carbohydrate Polymers 79 (2010) 423-428), the contents of which are incorporated herein by reference.

In some embodiments, the cationic moiety includes a nitrogen containing heterocyclic or heteroaromatic moiety (e.g, pyridinium, immidazolium, morpholinium, piperizinium, etc.). In some embodiments, the cationic polymer comprises a nitrogen containing heterocyclic or heteroaromatic moiety such as polyvinyl pyrrolidine or polyvinylpyrrolidinone.

In some embodiments, the cationic moiety includes a guanadinium moiety (e.g., an arginine moiety).

In some embodiments, the cationic moiety is a surfactant, for example, a cationic PVA such as a cationic PVA described herein.

Additional exemplary cationic moieties include agamatine, protamine sulfate, hexademethrine bromide, cetyl trimethylammonium bromide, 1-hexyltriethyl-ammonium phosphate, 1-dodecyltriethyl-ammonium phosphate, spermine (e.g., spermine tetrahydrochloride), spermidine, and derivatives thereof (e.g. N1-PLGA-spermine, N1-PLGA-N5,N10,N14-trimethylated-spermine, (N1-PLGA-N5,N10,N14, N14-tetramethylated-spermine), PLGA-glu-di-triCbz-spermine, triCbz-spermine, amiphipole, PMAL-C8, and acetyl-PLGA5050-glu-di(N1-amino-N5,N10,N14-spermine), poly(2-dimethylamino)ethyl methacrylate), hexyldecyltrimethylammonium chloride, hexadimethrine bromide, and atelocollagen and those described for example in WO2005007854, U.S. Pat. No. 7,641,915, and WO2009055445, the contents of each of which are incorporated herein by reference.

In an embodiment, a cationic moiety is one, the presence of which, in a particle described herein, is accompanied by the presence of less than 50, 40, 30, 20, or 10% (by weight or number) of the nucleic acid agent, e.g., siRNA, on the outside of the particle.

In an embodiment, the cationic moiety is not a lipid (e.g., not a phospholipid) or does not comprise a lipid.

Modes of Attachment

A nucleic acid or cationic moiety described herein may be directly (e.g., without the presence of atoms from an intervening spacer moiety), attached to a polymer or hydrophobic moiety described herein (e.g., a polymer). The attachment may be at a terminus of the polymer or along the backbone of the polymer. The nucleic acid, for example, when the nucleic acid is double stranded, can be attached to a polymer through the sense strand or the antisense strand. In some embodiments, the nucleic acid is modified at the point of attachment to the polymer; for example, a terminal hydroxy moiety of the nucleic acid (e.g., a 5′ or 3′ terminal hydroxyl moiety) is converted to a functional group that is reacted with the polymer (e.g., the hydroxyl moiety is converted to a thiol moiety). A reactive functional group of a nucleic acid or cationic moiety may be directly attached (e.g., without the presence of atoms from an intervening spacer moiety), to a functional group on a polymer. A nucleic acid or cationic moiety may be attached to a polymer via a variety of linkages, e.g., an amide, ester, sulfide (e.g., a maleimide sulfide), disulfide, succinimide, oxime, silyl ether, carbonate or carbamate linkage. For example, in one embodiment, a hydroxy group of a nucleic acid or cationic moiety may be reacted with a carboxylic acid group of a polymer, forming a direct ester linkage between the nucleic acid or cationic moiety and the polymer. In another embodiment, an amino group of a nucleic acid or cationic moiety may be linked to a carboxylic acid group of a polymer, forming an amide bond. In an embodiment a thiol modified nucleic acid may be reacted with a reactive moiety on the terminal end of the polymer (e.g., an acrylate PLGA, or a pyridinyl-SS-activated PLGA, or a maleimide activated PLGA) to form a sulfide or disulfide or thioether bond (i.e., sulfide bond). Exemplary modes of attachment include those resulting from click chemistry (e.g., an amide bond, an ester bond, a ketal, a succinate, or a triazole and those described in WO 2006/115547).

In some embodiments, a nucleic acid or cationic moiety may be directly attached (e.g., without the presence of atoms from an intervening spacer moiety), to a terminal end of a polymer. For example, a polymer having a carboxylic acid moiety at its terminus may be covalently attached to a hydroxy, thiol, or amino moiety of a nucleic acid or cationic moiety, forming an ester, thioester, or amide bond. In another embodiment, a nucleic acid or cationic moiety may be directly attached (e.g., without the presence of atoms from an intervening spacer moiety), along the backbone of a polymer. The nucleic acid, for example, when the nucleic acid is double stranded, can be attached to a polymer through the sense strand or the antisense strand.

In certain embodiments, suitable protecting groups may be required on the other polymer terminus or on other reactive substituents on the agent, to facilitate formation of the specific desired conjugate. For example, a polymer having a hydroxy terminus may be protected, e.g., with a silyl group (e.g., trimethylsilyl) or an acyl group (e.g., acetyl). A nucleic acid or cationic moiety may be protected, e.g., with an acetyl group or other protecting group.

In some embodiments, the process of attaching a nucleic acid or cationic moiety to a polymer may result in a composition comprising a mixture of conjugates having the same polymer and the same nucleic acid or cationic moiety, but which differ in the nature of the linkage between the nucleic acid or cationic moiety and the polymer. For example, when a nucleic acid or cationic moiety has a plurality of reactive moieties that may react with a polymer, the product of a reaction of the nucleic acid or cationic moiety and the polymer may include a conjugate wherein the nucleic acid or cationic moiety is attached to the polymer via one reactive moiety, and a conjugate wherein the nucleic acid or cationic moiety is attached to the polymer via another reactive moiety. For example, when a nucleic acid is attached to a polymer, the product of the reaction may include a nucleic acid-hydrophobic polymer conjugate where some of the nucleic acid is attached to the polymer through the 3′ end of the nucleic acid and some of the nucleic acid is attached to the polymer through the 5′ end of the nucleic acid. For example, when a nucleic acid having a double-stranded region is attached to a polymer, the product of the reaction may include a conjugate where some of the nucleic acid having a double-stranded region is attached to the polymer through the sense end and some of the nucleic acid having a double-stranded region is attached to the anti-sense end. Likewise, where a cationic moiety has multiple reactive groups such as a plurality of amines, the product of the reaction may include a conjugate where some of cationic moiety is attached to the polymer through a first reactive group and some of the cationic moiety is attached to the polymer through a second reactive group.

In some embodiments, the process of attaching a nucleic acid or cationic moiety to a polymer may involve the use of protecting groups. For example, when a nucleic acid or cationic moiety has a plurality of reactive moieties that may react with a polymer, the nucleic acid or cationic moiety may be protected at certain reactive positions such that a polymer will be attached via a specified position. In one embodiment, a nucleic acid or nucleic acid may be protected on the 3′ or 5′ end of the nucleic acid when attaching to a polymer. In one embodiment, a nucleic acid having a double-stranded region may be protected on the sense or anti-sense end when attaching to a polymer.

In some embodiments, selectively-coupled products such as those described above may be combined to form mixtures of polymer-agent conjugates. For example, PLGA attached to a nucleic acid through the 3′ end of the nucleic acid, and PLGA attached to a nucleic acid through the 5′ end of the nucleic acid may be combined to form a mixture of the two conjugates, and the mixture may be used in the preparation of a particle. In another embodiment, PLGA attached to an siRNA through the sense end (e.g., the 5′ end of the sense strand), and PLGA attached to an siRNA through the anti-sense end, may be combined to form a mixture of the two conjugates, and the mixture may be used in the preparation of a particle.

A nucleic acid-hydrophobic polymer conjugate may comprise a single nucleic acid or cationic moiety attached to a polymer. The nucleic acid or cationic moiety may be attached to a terminal end of a polymer, or to a point along a polymer chain.

In some embodiments, the conjugate may comprise a plurality of nucleic acids, e.g., nucleic acid agents or cationic moieties attached to a polymer (e.g., 2, 3, 4, 5, 6 or more agents may be attached to a polymer). The nucleic acids, e.g., nucleic acid agents or cationic moieties may be the same or different. In some embodiments, a plurality of nucleic acids, e.g., nucleic acid agents or cationic moieties may be attached to a multifunctional linker (e.g., a polyglutamic acid linker). In some embodiments, a plurality of nucleic acids, e.g., nucleic acid agents or cationic moieties may be attached to points along the polymer chain.

Linkers

A nucleic acid or cationic moiety may be attached to a moiety such as a polymer or a hydrophobic moiety such as a lipid, or to each other, via a linker, such as a linker described herein. For example: a hydrophobic polymer may be attached to a cationic moiety; a hydrophobic polymer may be attached to a nucleic acid agent; or a hydrophobic moiety may be attached to a cationic moiety. A nucleic acid may be attached to a moiety such as a polymer described herein through the 2′, 3′, or 5′ position of the nucleic acid, such as a terminal 2′, 3′, or 5′ position of the nucleic acid (e.g., through a linker described herein). In embodiments where the nucleic acid is double stranded (e.g., an siRNA), the nucleic acid can be attached through the sense or antisense strand. In some embodiments, the nucleic acid is attached through a terminal end of a polymer (e.g., a PLGA polymer, where the attachment is at the hydroxyl terminal or carboxy terminal).

In certain embodiments, a plurality of the linker moieties is attached to a polymer, allowing attachment of a plurality of nucleic acids, e.g., nucleic acid agents or cationic moieties to the polymer through linkers, for example, where the linkers are attached at multiple places on the polymer such as along the polymer backbone. In some embodiments, a linker is configured to allow for a plurality of a first moiety to be linked to a second moiety through the linker, for example, a plurality of nucleic acids, e.g., nucleic acid agents can be linked to a single polymer such as a PLGA polymer via a branched linker, wherein the branched linker comprises a plurality of functional groups through which the nucleic acid can be attached. In some embodiments, the nucleic acid or cationic moiety is released from the linker under biological conditions (i.e., cleavable under physiological conditions). In another embodiment a single linker is attached to a polymer, e.g., at a terminus of the polymer.

The linker may comprise, for example, an alkylene (divalent alkyl) group. In some embodiments, one or more carbon atoms of the alkylene linker may be replaced with one or more heteroatoms or functional groups (e.g., thioether, amino, ether, keto, amide, silyl ether, oxime, carbamate, carbonate, disulfide, or heterocyclic or heteroaromatic moieties). For example, an acrylate polymer (e.g., an acrylate PLGA) can be reacted with a thiol modified nucleic acid (e.g., a thiol modified siRNA) to form a nucleic acid-hydrophobic polymer conjugate attached through a sulfide bond (e.g., a thiopropionate linkage). The acrylate can be attached to a terminal end of the polymer (e.g., a hydroxyl terminal end of a PLGA polymer such as a 50:50 PLGA polymer) by reacting an acrylacyl chloride with the hydroxyl terminal end of the polymer.

In some embodiments, a linker, in addition to the functional groups that allow for attachment of a first moiety to a second moiety, has an additional functional group. In some embodiments, the additional functional group can be cleaved under physiological conditions. Such a linker can be formed, for example, by reacting a first activated moiety such as a nucleic acid agent or cationic moiety, e.g., a nucleic acid agent or cationic moiety described herein, with a second activated moiety such as a polymer, e.g., a polymer described herein, to produce a linker that includes a functional group that is formed by joining the nucleic acid agent or cationic moiety to the polymer. Optionally, the additional functional group can provide a site for additional attachments or allow for cleavage under physiological conditions. For example, the additional functional group may include a disulfide, ester, oxime, carbonate, carbamate, or amide bonds that are cleavable under physiological conditions. In some embodiments, one or both of the functional groups that attach the linker to the first or second moiety may be cleavable under physiological conditions such as esters, amides, or disulfides.

In some embodiments, the additional functional group is a heterocyclic or heteroaromatic moiety.

A nucleic acid may be attached through a linker (e.g., a linker comprising two or three functional groups such as a linker described herein) to a moiety such as a polymer described herein through the 2′, 3′, or 5′ position of the nucleic acid, such as a terminal 2′, 3′, or 5′ position of the nucleic acid. In embodiments where the nucleic acid is double stranded (e.g., an siRNA), the nucleic acid can be attached through the sense or antisense strand. In some embodiments, the nucleic acid is attached through a terminal end of a polymer (e.g., a PLGA polymer, where the attachment is at the hydroxyl terminal or carboxy terminal).

In some embodiments, the linker includes a moiety that can modulate the reactivity of a functional group in the linker (e.g., another functional group or atom that can increase or decrease the reactivity of a functional group, for example, under biological conditions).

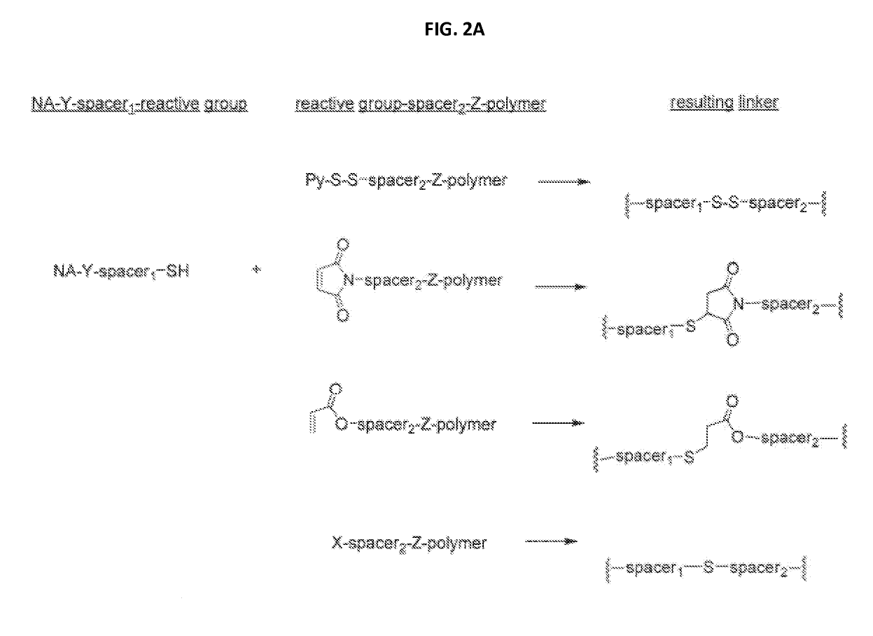

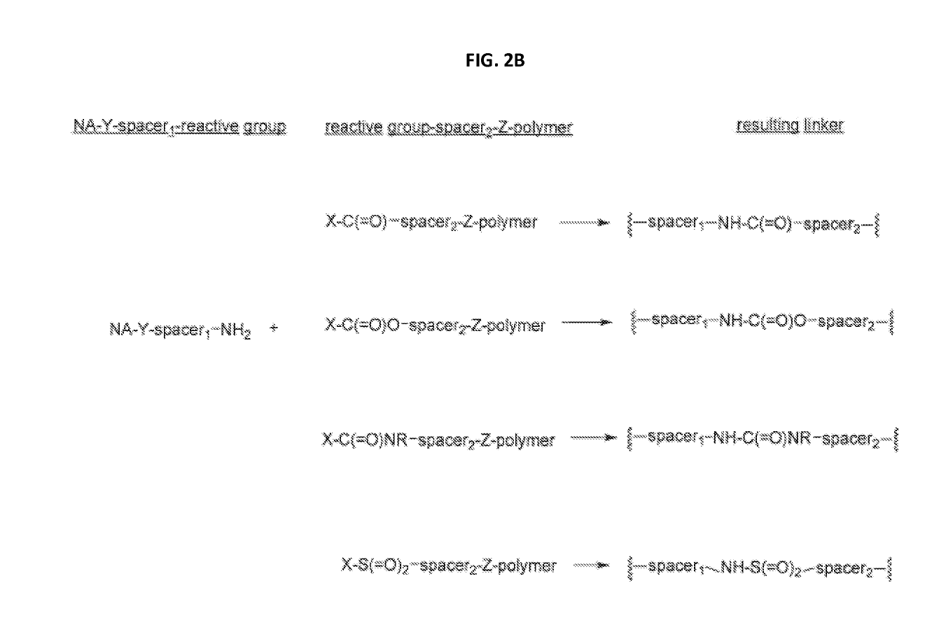

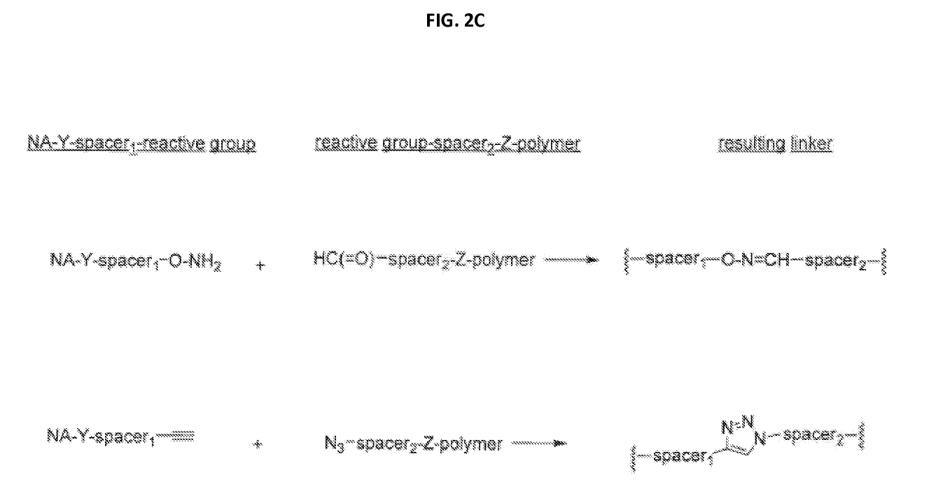

For example, as shown in FIGS. 2A-C, a nucleic acid (NA), e.g., RNA, having a first reactive group may be reacted with a polymer having a second reactive group to attach the nucleic acid to the polymer while providing a biocleavable functional group. The resulting linker includes a first spacer such as an alkylene spacer that attaches the nucleic acid to the functional group resulting from the attachment (i.e., by way of formation of a covalent bond), and a second spacer such as an alkylene spacer (e.g., from about C₁ to about C₆) that attaches the polymer to the functional group resulting from the attachment.

As shown in FIGS. 2A-C, the nucleic acid (NA) may be attached to the first spacer via a moiety Y, which also biocleavable. Y may be, for example, —O—, —S—, or —NH—. In some embodiments, the second spacer may be attached to a leaving group X—, for example halo (e.g., chloro) or N-hydroxysuccinimidyl (NHS). The second spacer may be attached to the polymer via an additional functional group (Z) that links with the polymer terminus, e.g., a terminal —OH, —CO₂H, —NH₂, or —SH, of a polymer, e.g., a terminal —OH or —CO₂H of PLGA. The additional functional group (Z) may be, for example, —O—, —OC(═O)—, —OC(═O)O—, —OC(═O)NR—, —NR—, —NRC(═O)—, —NRC(═O)O—, —NRC(═O)NR′—, —NRS(═O)₂—, —S—, —S(═O)—, —S(═O)₂—, —C(═O)O—, or —C(═O)NR—, and provides an additional site for reactivity, e.g., attachment or cleavage.

The nucleic acid may be attached through the 2′, 3′, or 5′ position of the nucleic acid, such as a terminal 2′, 3′, or 5′ position of the nucleic acid. In embodiments where the nucleic acid is double stranded (e.g., an siRNA), the nucleic acid can be attached through the sense or antisense strand. In some embodiments, the nucleic acid is attached through a spacer to the terminal end of a polymer (e.g., a PLGA polymer, where the attachment is at the hydroxyl terminal or carboxy terminal).

In an embodiment, e.g., as shown in FIG. 2A, a thiol modified nucleic acid (e.g., a thiol modified siRNA) can be reacted with a pyridynyl-SS-activated polymer (e.g., a pyridynyl-SS-activated PLGA, e.g., pyridynyl-SS-activated 5050 PLGA) to form a nucleic acid-hydrophobic polymer conjugate attached through a disulfide bond. In an embodiment, a thiol modified nucleic acid (e.g., a thiol modified siRNA) can be reacted with a maleimide-activated polymer (e.g., a maleimide-activated PLGA, e.g., maleimide-activated 5050 PLGA) to form a nucleic acid-hydrophobic polymer conjugate attached through a maleimide sulfide bond. In an embodiment, a thiol modified nucleic acid (e.g., a thiol modified siRNA) can be reacted with an acrylate-activated polymer (e.g., an acrylate-activated PLGA, e.g., acrylate-activated 5050 PLGA) to form a nucleic acid-hydrophobic polymer conjugate through a mercaptoproponate bond. The nucleic acid may be attached through the 2′, 3′, or 5′ position of the nucleic acid, such as a terminal 2′, 3′, or 5′ of the nucleic acid. In embodiments where the nucleic acid is double stranded (e.g., an siRNA), the nucleic acid can be attached through the sense or antisense strand. In some embodiments, the nucleic acid is attached through a spacer to the terminal end of a polymer (e.g., a PLGA polymer, where the attachment is at the hydroxyl terminal or carboxy terminal). In an embodiment, e.g., as shown in FIG. 2B, an amine modified nucleic acid (e.g., an amine modified siRNA) can be reacted with an polymer having an activated carboxylic acid or ester (e.g., an activated carboxylic acid PLGA, e.g., activated carboxylic acid 5050 PLGA, e.g., an SPA activated carboxylic acid PLGA, e.g., an SPA activated carboxylic acid 5050 PLGA) to form a nucleic acid-hydrophobic polymer conjugate attached through an amide bond. In an embodiment, an amine modified nucleic acid (e.g., an amine modified siRNA) can be reacted with an activated polymer (e.g., an activated PLGA, e.g., -activated 5050 PLGA) to form a nucleic acid-hydrophobic polymer conjugate attached through a carbamate bond. In an embodiment, an amine modified nucleic acid (e.g., an amine modified siRNA) can be reacted with an activated polymer (e.g., an activated PLGA, e.g., activated 5050 PLGA) to form a nucleic acid-hydrophobic polymer conjugate attached through a carbamide bond (urea). In an embodiment, an amine modified nucleic acid (e.g., an amine modified siRNA) can be reacted with an activated polymer (e.g., an activated PLGA, e.g., activated 5050 PLGA,) to form a nucleic acid-hydrophobic polymer conjugate attached through an aminoalkylsulfonamide bond. The nucleic acid may be attached through the 2′, 3′, or 5′ position of the nucleic acid, such as a terminal 2′, 3′, or 5′ of the nucleic acid. In embodiments where the nucleic acid is double stranded (e.g., an siRNA), the nucleic acid can be attached through the sense or antisense strand. In some embodiments, the nucleic acid is attached through a spacer to the terminal end of a polymer (e.g., a PLGA polymer, where the attachment is at the hydroxyl terminal or carboxy terminal).

In an embodiment, e.g., as shown in FIG. 2C, a hydroxylamine modified nucleic acid (e.g., a hydroxylamine modified siRNA) can be reacted with an aldehyde-activated polymer (e.g., an aldehyde-activated PLGA, e.g., aldehyde-activated 5050 PLGA, e.g., a formaldehyde-activated PLGA, e.g., formaldehyde-activated 5050 PLGA) to form a nucleic acid-hydrophobic polymer conjugate attached through an aldoxime bond. The nucleic acid may be attached through the 2′, 3′, or 5′ position of the nucleic acid, such as a terminal 2′, 3′, or 5′ of the nucleic acid. In embodiments where the nucleic acid is double stranded (e.g., an siRNA), the nucleic acid can be attached through the sense or antisense strand. In some embodiments, the nucleic acid is attached through a spacer to the terminal end of a polymer (e.g., a PLGA polymer, where the attachment is at the hydroxyl terminal or carboxy terminal).

In an embodiment, e.g., as shown in FIG. 2C, an alkylyne modified nucleic acid (e.g., an alkylyne modified siRNA, e.g., an acetylene modified siRNA) can be reacted with an azide-activated polymer (e.g., an azide-activated PLGA, e.g., azide-activated 5050 PLGA) to form a nucleic acid-hydrophobic polymer conjugate attached through a triazole bond. The nucleic acid may be attached through the 2′, 3′, or 5′ position of the nucleic acid, such as a terminal 2′, 3′, or 5′ of the nucleic acid. In embodiments where the nucleic acid is double stranded (e.g., an siRNA), the nucleic acid can be attached through the sense or antisense strand. In some embodiments, the nucleic acid is attached through a spacer to the terminal end of a polymer (e.g., a PLGA polymer, where the attachment is at the hydroxyl terminal or carboxy terminal).

In some embodiments, the linker, prior to attachment to the agent and the polymer, may have one or more of the following functional groups: amine, amide, hydroxyl, carboxylic acid, ester, halogen, thiol, maleimide, carbonate, or carbamate. In some embodiments, the functional group remains in the linker subsequent to the attachment of the first and second moiety through the linker. In some embodiments, the linker includes one or more atoms or groups that modulate the reactivity of the functional group (e.g., such that the functional group cleaves such as by hydrolysis or reduction under physiological conditions).

In some embodiments, the linker may comprise an amino acid or a peptide within the linker. Frequently, in such embodiments, the peptide linker is cleavable by hydrolysis, under reducing conditions, or by a specific enzyme (e.g., under physiological conditions).

When the linker is the residue of a divalent organic molecule, the cleavage of the linker may be either within the linker itself, or it may be at one of the bonds that couples the linker to the remainder of the conjugate, e.g., either to the nucleic acid or the polymer.

In some embodiments, a linker may be selected from one of the following or a linker may comprise one of the following:

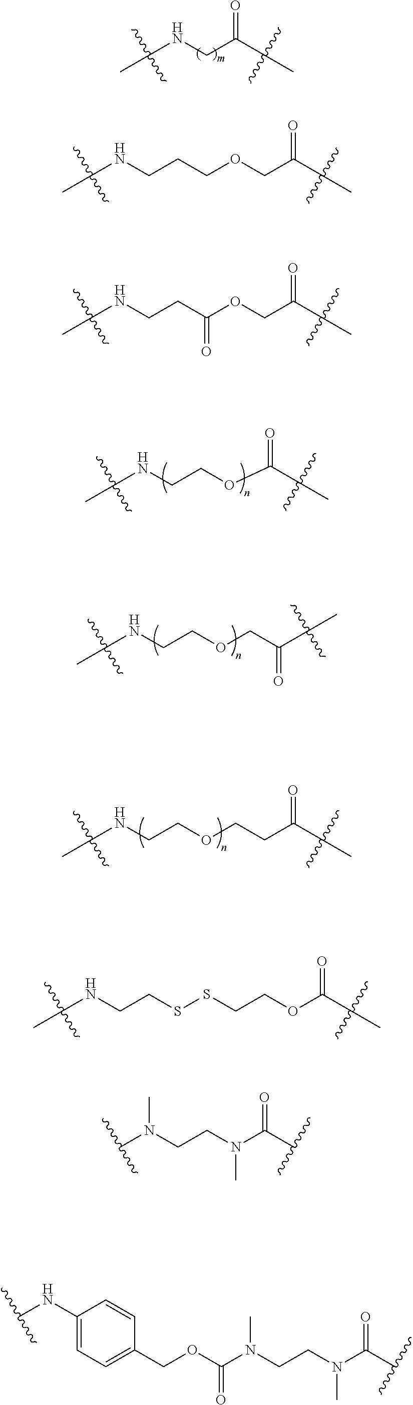

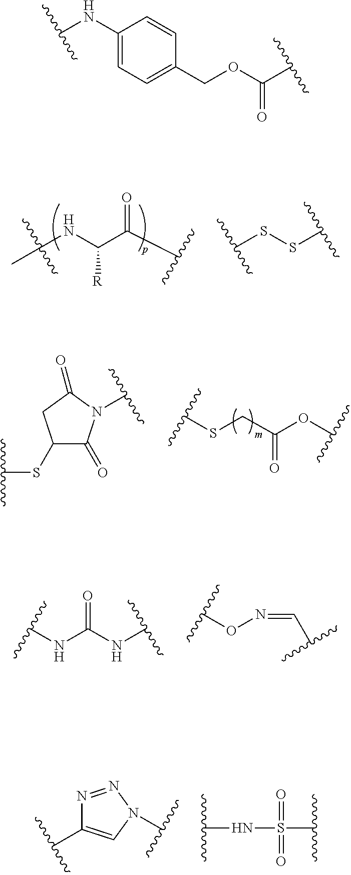

wherein m is 1-10, n is 1-10, p is 1-10, and R is an amino acid side chain.

A linker may include a bond resulting from click chemistry (e.g., an amide bond, an ester bond, a ketal, a succinate, or a triazole and those described in WO 2006/115547). A linker may be, for example, cleaved by hydrolysis, reduction reactions, oxidative reactions, pH shifts, photolysis, or combinations thereof; or by an enzyme reaction. The linker may also comprise a bond that is cleavable under oxidative or reducing conditions, or may be sensitive to acids.

In some embodiments, the linker is not cleaved under physiological conditions, for example, the linker is of a sufficient length that the nucleic acid does not need to be cleaved to be active, e.g., the length of the linker is at least about 20 angstroms (e.g., at least about 24 angstroms).

### Methods of Making Conjugates

Conjugates may be prepared using a variety of methods, including those described herein. In some embodiments, to covalently link the nucleic acid or cationic moiety to a polymer, the polymer or agent may be chemically activated using a technique known in the art. The activated polymer is then mixed with the agent, or the activated agent is mixed with the polymer, under suitable conditions to allow a covalent bond to form between the polymer and the agent. In some embodiments, a nucleophile, such as a thiol, hydroxyl group, or amino group, on the agent attacks an electrophile (e.g., activated carbonyl group) to create a covalent bond. A nucleic acid or cationic moiety may be attached to a polymer via a variety of linkages, e.g., an amide, ester, succinimide, carbonate or carbamate linkage.

In some embodiments, a nucleic acid or cationic moiety may be attached to a polymer via a linker. In such embodiments, a linker may be first covalently attached to a polymer, and then attached to a nucleic acid or cationic moiety. In other embodiments, a linker may be first attached to a nucleic acid or cationic moiety, and then attached to a polymer.

In some embodiments, where the method includes forming a nucleic acid-hydrophobic polymer conjugate such as a nucleic acid-hydrophobic polymer conjugate, the solubility of the nucleic acid and the polymer are significantly different. For example, the nucleic acid can be highly water soluble and the polymer (e.g., a hydrophobic polymer) can have low solubility (e.g., less than about 1 mg/mL). Such reactions can be done in a single solvent, or a solvent system comprising a plurality of solvents (e.g., miscible solvents). The solvent system can include water (e.g., an aqueous buffer system) and a polar solvent such as dimethylformamide (DMF), dimethylsulfoxide (DMSO), dimethylacetamine (DMA), N-methylpyrolydine (NMP), hexamethylphosphoramide (HMPA), fluoroisopropanol, trifluoroethanol, propylene carbonate, acetone, benzyl alcohol, dioxane, tetrahydrofuran (THF), or acetonitrile (e.g., ACN). Exemplary aqueous buffers include phosphate buffer solution (PBS), 4-(2-hydroxyethyl)-1-piperazineethanesulfonice acid (HEPES), TE buffer, or 2-(N-morpholino)ethanesulfonic acid buffer (MES)). The solvent system can be bi-phasic (e.g., having an organic and aqueous phase). In some embodiments, the ratio of polar solvent (e.g., “org”) to water (e.g., an aqueous buffer system) is from about 90/10 to about 40/60 (e.g., from about 80/10 to about 50/50, from about 80/10 to about 60/40, about 80/20, about 60/40 or about 50/50).

Exemplary solvent systems that can be used to attach a nucleic acid to a hydrophobic polymer include those in Table 2 below.

TABLE 2

| | 50/50 | 60/40 | 60/40 | 80/20 | 80/20 |
|---|---|---|---|---|---|
| Solvent | Org*/PBS** | Org/TE*** | Org/PBS | Org/TE | Org/PBS |
| DMSO | Translucent | TranslucentSome | Turbid | Translucent | Translucent |
| | Some ppt. | ppt. | | | |
| Acetonitrile | Translucent | Milky | Translucent | Clear | Clear |
| | oil droplets | | Some tiny oil | | |
| | | | droplets | | |
| Acetone | Translucent | Milky | Translucent | Milky | Translucent |
| | Some tiny oil | | Some tiny oil | | |
| | droplets | | droplets | | |
| THF | Translucent | Milky | Translucent | Translucent | Translucent |
| | Some tiny oil | | Some tiny oil | | |
| | droplets | | droplets | | |
| DMF | Milky | Milky | Milky | Milky | Translucent |
| | | | | | w/ ppt |

The above table is for a concentration of 10 mg/mL polymer.
*Org refers to an organic solvent.
**TE refers to an aqueous buffer solution having TE as the buffer (i.e., 1 mM Tris, brrought to pH 8.0 with HCl, and 1 mM EDTA)
***PBS refers to an aqueous buffer solution having PBS as the buffer (i.e., phosphate buffered saline.

The methods described herein can be done using an excess of one or more reagents. For example, when forming a nucleic acid-hydrophobic polymer conjugate, the reaction can be performed using an excess of either the hydrophobic polymer or the nucleic acid.

The methods described herein can be performed where at least one of the nucleic acid or hydrophobic polymer is attached to an insoluble substrate (e.g., the polymer).

The methods described herein can result in a nucleic acid-hydrophobic polymer conjugate having a purity of at least about 80% (e.g., at least about 85%, at least about 90%, at least about 95%, at least about 99%). In some embodiments, method produces at least about 100 mg of the nucleic acid-hydrophobic polymer conjugate (e.g., at least about 1 g).

### Compositions of Conjugates

Compositions of nucleic acid-hydrophobic polymer conjugates described above (e.g., nucleic acid agent-hydrophobic polymer conjugates or cationic moiety-polymer conjugates) may include mixtures of products. For example, the conjugation of a nucleic acid agent or cationic moiety to a polymer may proceed in less than 100% yield, and the composition comprising the conjugate may thus also include unconjugated hydrophobic polymer, unconjugated nucleic acid agent, and/or unconjugated cationic moiety.

Compositions of conjugates (nucleic acid-hydrophobic polymer conjugates) may also include conjugates that have the same polymer and the same nucleic acid agent and/or cationic moiety, and differ in the nature of the linkage between the nucleic acid agent and/or cationic moiety and the polymer. For example, in some embodiments, when the conjugate is a nucleic acid-hydrophobic polymer conjugate, the composition may include polymers attached to the nucleic acid agent via different hydroxyl groups present on the nucleic acid agent (e.g., the 2′, 3′, or 5′ hydroxyl groups such as the 3′ or 5′). The nucleic acid-hydrophobic polymer conjugates may be present in the composition in varying amounts. For example, when a nucleic acid agent and/or cationic moiety having a plurality of available attachment points is reacted with a polymer, the resulting composition may include more of a product conjugated via a more reactive group (e.g., a first hydroxyl or amino group), and less of a product attached via a less reactive group (e.g., a second hydroxyl or amino group).

Additionally, compositions of nucleic acid-hydrophobic polymer conjugates may include nucleic acid agents and/or cationic moieties that are attached to more than one polymer chain. For example, in the case of a nucleic acid-hydrophobic polymer conjugates, the nucleic acid may be attached to a first polymer chain through a 3′ hydroxyl and a second polymer chain through a 5′ hydroxyl.

### Particles

In another aspect, the particles described herein can include a nucleic acid agent, a first element, e.g., a hydrophobic-hydrophilic polymer, and a second element, e.g., a surfactant. An exemplary particle includes a particle comprising:

a) a plurality of hydrophobic polymers;

b) a plurality of hydrophilic-hydrophobic polymers;

c) optionally, a plurality of cationic moieties; and

d) a plurality of nucleic acid agents wherein at least a portion of the plurality of nucleic acid agents are

(i) covalently attached to a hydrophobic polymer of a), or

(ii) form a duplex (e.g., a heteroduplex) with a nucleic acid which is covalently attached to a hydrophobic polymer of a).

In some embodiments, the particles include a nucleic acid agent and a cationic moiety, and at least one of a hydrophobic moiety, such as a polymer, or a hydrophilic-hydrophobic polymer. In some embodiments, a particle described herein includes a hydrophobic moiety such as a hydrophobic polymer or lipid (e.g., hydrophobic polymer), a polymer containing a hydrophilic portion and a hydrophobic portion, a nucleic acid agent, and a cationic moiety. In some embodiments, the nucleic acid agent and/or cationic moiety is attached to a moiety. For example, the nucleic acid agent and/or cationic moiety can be attached to a polymer (e.g., the hydrophobic polymer) or the nucleic acid agent forms a duplex with a nucleic acid that is attached to a polymer. In some embodiments, the nucleic acid agent is attached to a polymer (e.g., a hydrophobic polymer), and the cationic moiety is not attached to a polymer (e.g., the cationic moiety is embedded in the particle). In some embodiments, the nucleic acid agent and the cationic moiety are both attached to a polymer (e.g., a hydrophobic polymer or a polymer containing a hydrophilic and a hydrophobic portion) or the nucleic acid agent forms a duplex with a nucleic acid that is attached to a polymer and the cationic moiety is attached to a polymer. In some embodiments, the cationic moiety is attached to a polymer (e.g., a hydrophobic polymer or a polymer containing a hydrophilic and a hydrophobic portion).

In addition to a hydrophobic moiety such as a hydrophobic polymer or lipid (e.g., hydrophobic polymer), a polymer containing a hydrophilic portion and a hydrophobic portion, a nucleic acid agent, and a cationic moiety, the particles described herein may include one or more additional components such as an additional nucleic acid agent or an additional cationic moiety. A particle described herein may also include a compound having at least one acidic moiety, such as a carboxylic acid group. The compound may be a small molecule or a polymer having at least one acidic moiety. In some embodiments, the compound is a polymer such as PLGA.

In some embodiments, the particle is configured such that when administered to a subject there is preferential release of the nucleic acid agent, e.g., siRNA, in a preselected compartment. The preselected compartment can be a target site, location, tissue type, cell type, e.g., a disease specific cell type, e.g., a cancer cell, or subcellular compartment, e.g., the cytosol. In an embodiment a particle provides preferential release in a tumor, as opposed to other compartments, e.g., non-tumor compartments, e.g., the peripheral blood. In embodiments, where the nucleic acid agent, e.g., an siRNA, is attached to a polymer or a cationic moiety, the nucleic acid agent is released (e.g., through reductive cleavage of a linker) to a greater degree in a tumor than in non-tumor compartments, e.g., the peripheral blood, of a subject. In some embodiments, the particle is configured such that when administered to a subject, it delivers more nucleic acid agent, e.g, siRNA, to a compartment of the subject, e.g., a tumor, than if the nucleic acid agent were administered free.

In some embodiments, the particle is associated with an excipient, e.g., a carbohydrate component, or a stabilizer or lyoprotectant, e.g., a carbohydrate component, stabilizer or lyoprotectant described herein. While not wishing to be bound be theory the carbohydrate component may act as a stabilizer or lyoprotectant. In some embodiments, the carbohydrate component, stabilizer or lyoprotectant, comprises one or more carbohydrates (e.g., one or more carbohydrates described herein, such as, e.g., sucrose, cyclodextrin or a derivative of cyclodextrin (e.g. 2-hydroxypropyl-β-cyclodextrin, sometimes referred to herein as HP-β-CD)), salt, PEG, PVP or crown ether. In some embodiments, the carbohydrate component, stabilizer or lyoprotectant comprises two or more carbohydrates, e.g., two or more carbohydrates described herein. In one embodiment, the carbohydrate component, stabilizer or lyoprotectant includes a cyclic carbohydrate (e.g., cyclodextrin or a derivative of cyclodextrin, e.g., an α-, β-, or γ-, cyclodextrin (e.g. 2-hydroxypropyl-β-cyclodextrin)) and a non-cyclic carbohydrate. Exemplary non-cyclic oligosaccharides include those of less than 10, 8, 6 or 4 monosaccharide subunits (e.g., a monosaccharide or a disaccharide (e.g., sucrose, trehalose, lactose, maltose) or combinations thereof).

In an embodiment the carbohydrate component, stabilizer or lyoprotectant comprises a first and a second component, e.g., a cyclic carbohydrate and a non-cyclic carbohydrate, e.g., a mono-, di, or tetra saccharide.

In one embodiment, the weight ratio of cyclic carbohydrate to non-cyclic carbohydrate associated with the particle is a weight ratio described herein, e.g., 0.5:1.5 to 1.5:0.5.

In an embodiment the carbohydrate component, stabilizer or lyoprotectant comprises a first and a second component (designated here as A and B) as follows:
(A) comprises a cyclic carbohydrate and (B) comprises a disaccharide;
(A) comprises more than one cyclic carbohydrate, e.g., a β-cyclodextrin (sometimes referred to herein as β-CD) or a β-CD derivative, e.g., HP-β-CD, and (B) comprises a disaccharide;
(A) comprises a cyclic carbohydrate, e.g., a β-CD or a β-CD derivative, e.g., HP-β-CD, and (B) comprises more than one disaccharide;
(A) comprises more than one cyclic carbohydrate, and (B) comprises more than one disaccharide;
(A) comprises a cyclodextrin, e.g., a β-CD or a β-CD derivative, e.g., HP-β-CD, and (B) comprises a disaccharide;
(A) comprises a β-cyclodextrin, e.g a β-CD derivative, e.g., HP-β-CD, and (B) comprises a disaccharide;
(A) comprises a β-cyclodextrin, e.g., a β-CD derivative, e.g., HP-β-CD, and (B) comprises sucrose;
(A) comprises a β-CD derivative, e.g., HP-β-CD, and (B) comprises sucrose;
(A) comprises a β-cyclodextrin, e.g., a β-CD derivative, e.g., HP-β-CD, and (B) comprises trehalose;
(A) comprises a β-cyclodextrin, e.g., a β-CD derivative, e.g., HP-β-CD, and (B) comprises sucrose and trehalose.
(A) comprises HP-β-CD, and (B) comprises sucrose and trehalose.

In an embodiment components A and B are present in the following ratio:

0.5:1.5 to 1.5:0.5. In an embodiment, components A and B are present in the following ratio: 3-1:0.4-2; 3-1:0.4-2.5; 3-1:0.4-2; 3-1:0.5-1.5; 3-1:0.5-1; 3-1:1; 3-1:0.6-0.9; and 3:1:0.7. In an embodiment, components A and B are present in the following ratio: 2-1:0.4-2; 3-1:0.4-2.5; 2-1:0.4-2; 2-1:0.5-1.5; 2-1:0.5-1; 2-1:1; 2-1:0.6-0.9; and 2:1:0.7. In an embodiment components A and B are present in the following ratio: 2-1.5:0.4-2; 2-1.5:0.4-2.5; 2-1.5:0.4-2; 2-1.5:0.5-1.5; 2-1.5:0.5-1; 2-1.5:1; 2-1.5:0.6-0.9; 2:1.5:0.7. In an embodiment components A and B are present in the following ratio: 2.5-1.5:0.5-1.5; 2.2-1.6:0.7-1.3; 2.0-1.7:0.8-1.2; 1.8:1; 1.85:1 and 1.9:1.

In an embodiment component A comprises a cyclodextin, e.g., a β-cyclodextrin, e.g., a β-CD derivative, e.g., HP-β-CD, and (B) comprises sucrose, and they are present in the following ratio: 2.5-1.5:0.5-1.5; 2.2-1.6:0.7-1.3; 2.0-1.7:0.8-1.2; 1.8:1; 1.85:1 and 1.9:1.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm). In an embodiment, the nanoparticle has a diameter of at least 10 nm (e.g., at least about 20 nm).

A particle described herein may also include a targeting agent or a lipid (e.g., on the surface of the particle).

A composition of a plurality of particles described herein may have an average diameter of about 50 nm to about 500 nm (e.g., from about 50 nm to about 200 nm). A composition of a plurality of particles particle may have a median particle size (Dv50 (particle size below which 50% of the volume of particles exists) of about 50 nm to about 500 nm (e.g., about 75 nm to about 220 nm)) from about 50 nm to about 220 nm (e.g., from about 75 nm to about 200 nm). A composition of a plurality of particles may have a Dv90 (particle size below which 90% of the volume of particles exists) of about 50 nm to about 500 nm (e.g., about 75 nm to about 220 nm). In some embodiments, a composition of a plurality of particles has a Dv90 of less than about 150 nm. A composition of a plurality of particles may have a particle PDI of less than 0.5, less than 0.4, less than 0.3, less than 0.2, or less than 0.1.

A particle described herein may have a surface zeta potential ranging from about −20 mV to about 50 mV, when measured in water. Zeta potential is a measurement of surface potential of a particle. In some embodiments, a particle may have a surface zeta potential, when measured in water, ranging between about −20 mV to about 20 mV, about −10 mV to about 10 mV, or neutral.

In an embodiment, a particle, or a composition comprising a plurality of particles, described herein, has a sufficient amount of nucleic acid agent (e.g., an siRNA), to observe an effect (e.g., knock-down) when administered, for example, in an in vivo model system, (e.g., a mouse model such as any of those described herein).

In an embodiment, a particle, or a composition comprising a plurality of particles described herein, is one in which at least 30, 40, 50, 60, 70, 80, or 90% of its nucleic acid agent, e.g., siRNA, by number or weight, is intact (e.g., as measured by functionality of physical properties, e.g., molecular weight).

In an embodiment, a particle, or a composition comprising a plurality of particles, described herein, is one in which at least 30, 40, 50, 60, 70, 80, or 90% of its nucleic acid agent, e.g., siRNA, by number or weight, is inside, as opposed to exposed at the surface of, the particle.

In an embodiment, a particle, or a composition comprising a plurality of particles, described herein, when incubated in 50/50 mouse/human serum, exhibits little or no aggregation. E.g., when incubated less than 30, 20, or 10%, by number or weight, of the particles will aggregate.

In an embodiment, a particle, or a composition comprising a plurality of particles, described herein may, when stored at 25° C.±2° C./60% relative humidity±5% relative humidity in an open, or closed, container, for 20, 30, 40, 50 or 60 days, retains at least 30, 40, 50, 60, 70, 80, 90, or 95% of its activity, e.g., as determined in an in vivo model system, (e.g., a mouse model such any of those described herein).

In an embodiment, a particle, or a composition comprising a plurality of particles, described herein may, results in at least 20, 30, 40, 50, or 60% reduction in protein and/or mRNA knockdown when administered as a single dose of 1 or 3 mg/kg in an in vivo model system, (e.g., a mouse model such as any of those described herein).

In an embodiment, a particle or a composition comprising a plurality of particles described herein results in less than 20, 10, 5%, or no knockdown for off target genes, as measured by protein or mRNA, when administered (e.g., as a single dose of 1 or 3 mg/kg) in an in vivo model system, (e.g., a mouse model such as any of those described herein).

In some embodiments, the particles described herein can deliver an effective amount of the nucleic acid agent such that expression of the targeted gene in the subject is reduced by at least 20%, 25%, 30%, 35%, 40%, 45%, 50%, 55%, 60%, 65%, 70%, 75%, 80%, 85%, 90%, 95% or more at approximately 72 hours, 96 hours, 120 hours, 144 hours, 168 hours, 192 hours, 216 hours, 240 hours, 264 hours after administration of the particles to the subject. In one embodiment, the particles described herein can deliver an effective amount of the nucleic acid agent such that expression of the targeted gene in the subject is reduced by at least 50%, 55%, 60%, 65%, 70%, 75% or 80%, approximately 120 hours after administration of the particles to the subject. In some embodiments, the level of target gene expression in a subject administered a particle or composition described herein is compared to the level of expression of the target gene seen when the nucleic acid agent is administered in a formulation other than a particle or a conjugate (i.e., not in a particle, e.g., not embedded in a particle or conjugated to a polymer, for example, a particle described herein) or than expression of the target gene seen in the absence of the administration of the nucleic acid agent or other therapeutic agent).

In an embodiment, a particle or a composition comprising a plurality of particles, described herein, when contacted with target gene mRNA, results in cleavage of the mRNA.

In an embodiment, a particle or a composition comprising a plurality of particles, described herein, results in less than 2, 5, or 10 fold cytokine induction, when administered (e.g., as a single dose of 1 or 3 mg/kg) in an in vivo model system, (e.g., a mouse model such as any of those described herein). E.g., the administration results in less than 2, 5, or 10 fold induction of one, or more, e.g., two, three, four, five, six, or seven, or all, of: tumor necrosis factor-alpha, interleukin-1alpha, interleukin-1beta, interleukin-6, interleukin-10, interleukin-12, keratinocyte-derived cytokine and interferon-gamma.

In an embodiment, a particle, or a composition comprising a plurality of particles, described herein, results in less than 2, 5, or 10 fold increase in alanine aminotransferase (ALT) and aspartate aminotransferase (AST), when administered (e.g., as a single dose of 1 or 3 mg/kg) in an in vivo model system (e.g., a mouse model such as any of those described herein). In an embodiment, a particle, or a composition comprising a plurality of particles, described herein, results in no significant changes in blood count 48 hours after 2 doses of 3 mg/kg in an in vivo model system, (e.g., a mouse model such as one described herein).

In an embodiment a particle is stable in non-polar organic solvent (e.g., any of hexane, chloroform, or dichloromethane). By way of example, the particle does not substantially invert, e.g., if present, an outer layer does not internalize, or a substantial amount of surface components do internalize, relative to their configuration in aqueous solvent. In embodiments the distribution of components is substantially the same in a non-polar organic solvent and in an aqueous solvent.

In an embodiment a particle lacks at least one component of a micelle, e.g., it lacks a core which is substantially free of hydrophilic components.

In an embodiment the core of the particle comprises a substantial amount of a hydrophilic component.

In an embodiment the core of the particle comprises a substantial amount e.g., at least 1, 2, 3, 4, 5, 6, 7, 8, 9, 10, 20, 30, 40, 50, 60 or 70% (by weight or number) of the nucleic acid agent, e.g., siRNA, of the particle.

In an embodiment the core of the particle comprises a substantial amount e.g., at least 10, 20, 30, 40, 50, 60 or 70% (by weight or number) of the cationic, e.g., polycationic moiety, of the particle.

A particle described herein may include a small amount of a residual solvent, e.g., a solvent used in preparing the particles such as acetone, tert-butylmethyl ether, benzyl alcohol, dioxane, heptane, dichloromethane, dimethylformamide, dimethylsulfoxide, ethyl acetate, acetonitrile, tetrahydrofuran, ethanol, methanol, isopropyl alcohol, methyl ethyl ketone, butyl acetate, or propyl acetate (e.g., isopropylacetate). In some embodiments, the particle may include less than 5000 ppm of a solvent (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm).

In some embodiments, the particle is substantially free of a class II or class III solvent as defined by the United States Department of Health and Human Services Food and Drug Administration “Q3c—Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

A particle described herein may include varying amounts of a hydrophobic moiety such as a hydrophobic polymer, e.g., from about 20% to about 90% by weight of, or used as starting materials to make, the particle (e.g., from about 20% to about 80%, from about 25% to about 75%, or from about 30% to about 70% by weight).

A particle described herein may include varying amounts of a polymer containing a hydrophilic portion and a hydrophobic portion, e.g., up to about 50% by weight of, or used as starting materials to make, the particle (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the hydrophobic-hydrophilic polymer of the particle is from about 3% to 30%, from about 5% to 25% or from about 8% to 23%.

In a particle described herein, the ratio of the hydrophobic polymer to the hydrophobic-hydrophilic polymer is such that the particle comprises at least 5%, 8%, 10%, 12%, 15%, 18%, 20%, 23%, 25%, or 30% by weight of a polymer of, or used as starting materials to make, the particle having a hydrophobic portion and a hydrophilic portion.

A particle described herein may include varying amounts of a cationic moiety, e.g., from about 0.1% to about 90% by weight of, or used as starting materials to make, the particle (e.g., from about 1% to about 60%, from about 2% to about 20%, from about 3% to about 30%, from about 5% to about 40%, from about or from about 10% to about 30%). When the cationic moiety is a nitrogen containing moiety, the ratio of nitrogen moieties in the particle to phosphates from the nucleic acid agent backbone in the particle (i.e., N/P ratio) can be from about 1:1 to about 50:1 (e.g., from about 1:1 to about 25:1, from about 1:1 to about 10:1, from about 1:1 to about 5:1, or from about 1:1 to about 1.5 to 1:1).

A particle described herein may include varying amounts of a nucleic acid agent, e.g., from about 0.1% to about 50% by weight of, or used as starting materials to make, the particle (e.g., from about 1% to about 50%, from about 0.5% to about 20%, from about 2% to about 20%, from about or from about 5% to about 15%).

When the particle includes a surfactant, the particle may include varying amounts of the surfactant, e.g., up to about 40% by weight of, or used as starting materials to make, the particle, or from about 15% to about 35% or from about 3% to about 10%. In some embodiments, the surfactant is PVA and the cationic moiety is cationic PVA. In some embodiments, the particle may include about 2% to about 5% of PVA (e.g., about 4%) and from about 0.1% to about 3% cationic PVA (e.g., about 1%).

A particle described herein may be substantially free of a targeting agent (e.g., of a targeting agent covalently linked to a component in the particle, e.g., a targeting agent able to bind to or otherwise associate with a target biological entity, e.g., a membrane component, a cell surface receptor, prostate specific membrane antigen, or the like). A particle described herein may be substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer within the particle is conjugated to a targeting moiety. A particle described herein may be free of moieties added for the purpose of selectively targeting the particle to a site in a subject, e.g., by the use of a moiety on the particle having a high and specific affinity for a target in the subject.

In some embodiments the particle is free of a lipid, e.g., free of a phospholipid. A particle described herein may be substantially free of an amphiphilic layer that reduces water penetration into the nanoparticle. A particle described herein may comprise less than 5 or 10% (e.g., as determined as w/w, v/v) of a lipid, e.g., a phospholipid. A particle described herein may be substantially free of a lipid layer, e.g., a phospholipid layer, e.g., that reduces water penetration into the nanoparticle. A particle described herein may be substantially free of lipid, e.g., is substantially free of phospholipid.

A particle described herein may be substantially free of a radiopharmaceutical agent, e.g., a radiotherapeutic agent, radiodiagnostic agent, prophylactic agent, or other radioisotope. A particle described herein may be substantially free of an immunomodulatory agent, e.g., an immunostimulatory agent or immunosuppressive agent. A particle described herein may be substantially free of a vaccine or immunogen, e.g., a peptide, sugar, lipid-based immunogen, B cell antigen or T cell antigen.

A particle described herein may be substantially free of a water-soluble hydrophobic polymer such as PLGA, e.g., PLGA having a molecular weight of less than about 1 kDa (e.g., less than about 500 Da).

### Additional Elements

In some embodiments, the particle further comprises a surfactant or a mixture of surfactants. In some embodiments, the surfactant is PEG, poly(vinyl alcohol) (PVA), poly(vinylpyrrolidone) (PVP), poloxamer, hexyldecyltrimethylammonium chloride, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)], lecithin, or a mixture thereof. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the PVA has a viscosity of from about 2 to about 27 cP. In some embodiments, the PVA is a cationic PVA, for example, as described above, for example, a cationic moiety such as a cationic PVA can also serve as a surfactant. In some embodiments, the surfactant is polysorbate 80. In some embodiments, the surfactant is Solutol® HS 15. In some embodiments, the surfactant is not a lipid (e.g., a phospholipid) or does not comprise a lipid. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle is associated with an excipient, e.g., a carbohydrate component, or a stabilizer or lyoprotectant, e.g., a carbohydrate component, stabilizer or lyoprotectant described herein. While not wishing to be bound be theory the carbohydrate component may act as a stabilizer or lyoprotectant. In some embodiments, the carbohydrate component, stabilizer or lyoprotectant, comprises one or more sugars, sugar alcohols, carbohydrates (e.g., sucrose, mannitol, cyclodextrin or a derivative of cyclodextrin (e.g. 2-hydroxypropyl-β-cyclodextrin, sometimes referred to herein as HP-β-CD, or sulfobutyl-CD, sometimes referred to herein as CYTOSOL.)), salt, PEG, PVP or crown ether. In some embodiments, the carbohydrate component, stabilizer or lyoprotectant comprises two or more carbohydrates, e.g., two or more carbohydrates described herein. In one embodiment, the carbohydrate component, stabilizer or lyoprotectant includes a cyclic carbohydrate (e.g., cyclodextrin or a derivative of cyclodextrin, e.g., an α-, β-, or γ-, cyclodextrin (e.g. 2-hydroxypropyl-β-cyclodextrin)) and a non-cyclic carbohydrate. Exemplary non-cyclic oligosaccharides include those of less than 10, 8, 6 or 4 monosaccharide subunits (e.g., a monosaccharide or a disaccharide (e.g., sucrose, trehalose, lactose, maltose) or combinations thereof). In some embodiments, the lyoprotectant is a monosaccharide such as a sugar alcohol (e.g., mannitol).

In an embodiment the carbohydrate element, stabilizer or lyoprotectant comprises a first and a second component, e.g., a cyclic carbohydrate and a non-cyclic carbohydrate, e.g., a mono-, di, or tetra saccharide.

In one embodiment, the weight ratio of cyclic carbohydrate to non-cyclic carbohydrate associated with the particle is a weight ratio described herein, e.g., 0.5:1.5 to 1.5:0.5.

In an embodiment the carbohydrate element, stabilizer or lyoprotectant comprises a first and a second element (designated here as A and B) as follows:
(A) comprises a cyclic carbohydrate and (B) comprises a disaccharide;
(A) comprises more than one cyclic carbohydrate, e.g., a β-cyclodextrin (sometimes referred to herein as β-CD) or a β-CD derivative, e.g., HP-β-CD, and (B) comprises a disaccharide;
(A) comprises a cyclic carbohydrate, e.g., a β-CD or a β-CD derivative, e.g., HP-β-CD, and (B) comprises more than one disaccharide;
(A) comprises more than one cyclic carbohydrate, and (B) comprises more than one disaccharide;
(A) comprises a cyclodextrin, e.g., a β-CD or a β-CD derivative, e.g., HP-β-CD, and (B) comprises a disaccharide;
(A) comprises a β-cyclodextrin, e.g a β-CD derivative, e.g., HP-β-CD, and (B) comprises a disaccharide;
(A) comprises a β-cyclodextrin, e.g., a β-CD derivative, e.g., HP-β-CD, and (B) comprises sucrose;
(A) comprises a β-CD derivative, e.g., HP-β-CD, and (B) comprises sucrose;
(A) comprises a β-cyclodextrin, e.g., a β-CD derivative, e.g., HP-β-CD, and (B) comprises trehalose;
(A) comprises a β-cyclodextrin, e.g., a β-CD derivative, e.g., HP-β-CD, and (B) comprises sucrose and trehalose.
(A) comprises HP-β-CD, and (B) comprises sucrose and trehalose.

In an embodiment elements A and B are present in the following ratio: 0.5:1.5 to 1.5:0.5. In an embodiment, elements A and B are present in the following ratio: 3-1:0.4-2; 3-1:0.4-2.5; 3-1:0.4-2; 3-1:0.5-1.5; 3-1:0.5-1; 3-1:1; 3-1:0.6-0.9; and 3:1:0.7. In an embodiment, components A and B are present in the following ratio: 2-1:0.4-2; 3-1:0.4-2.5; 2-1:0.4-2; 2-1:0.5-1.5; 2-1:0.5-1; 2-1:1; 2-1:0.6-0.9; and 2:1:0.7. In an embodiment components A and B are present in the following ratio: 2-1.5:0.4-2; 2-1.5:0.4-2.5; 2-1.5:0.4-2; 2-1.5:0.5-1.5; 2-1.5:0.5-1; 2-1.5:1; 2-1.5:0.6-0.9; 2:1.5:0.7. In an embodiment components A and B are present in the following ratio: 2.5-1.5:0.5-1.5; 2.2-1.6:0.7-1.3; 2.0-1.7:0.8-1.2; 1.8:1; 1.85:1 and 1.9:1.

In an embodiment element A comprises a cyclodextrin, e.g., a β-cyclodextrin, e.g., a β-CD derivative, e.g., HP-β-CD, and (B) comprises sucrose, and they are present in the following ratio: 2.5-1.5:0.5-1.5; 2.2-1.6:0.7-1.3; 2.0-1.7:0.8-1.2; 1.8:1; 1.85:1 and 1.9:1.

In some embodiments, the surface of the particle can be substantially coated with a surfactant or polymer, for example, PVA, polyoxazoline, polyvinylpyrrolidine, polyhydroxylpropylmethacrylamide, polysialic acid, or PEG.

### Methods of Making Particles and Compositions

A particle described herein may be prepared using any method known in the art for preparing particles, e.g., nanoparticles. Exemplary methods include spray drying, emulsion (e.g., emulsion-solvent evaporation or double emulsion), precipitation (e.g., nanoprecipitation) and phase inversion.

In one embodiment, a particle described herein can be prepared by precipitation (e.g., nanoprecipitation). This method involves dissolving the components of the particle (i.e., one or more polymers, an optional additional component or components, a cationic moiety and a nucleic acid agent), individually or combined, in one or more solvents to form one or more solutions. For example, a first solution containing one or more of the components may be poured into a second solution containing one or more of the components (at a suitable rate or speed). The solutions may be combined, for example, using a syringe pump, a MicroMixer, or any device that allows for vigorous, controlled mixing. In some cases, nanoparticles can be formed as the first solution contacts the second solution, e.g., precipitation of the polymer upon contact causes the polymer to form nanoparticles. The control of such particle formation can be readily optimized.

In one set of embodiments, the particles are formed by providing one or more solutions containing one or more polymers and additional components, and contacting the solutions with certain solvents to produce the particle. In a non-limiting example, a hydrophobic polymer (e.g., PLGA), is conjugated to a nucleic acid agent or cationic moiety to form a nucleic acid-hydrophobic polymer conjugate. This nucleic acid-hydrophobic polymer conjugate, a polymer containing a hydrophilic portion and a hydrophobic portion (e.g., PEG-PLGA), nucleic acid agent and/or cationic moiety, and optionally a third polymer (e.g., a biodegradable polymer, e.g., PLGA) are dissolved in a partially water miscible organic solvent (e.g., acetone). This solution is added to an aqueous solution containing a surfactant, forming the desired particles. These two solutions may be individually sterile filtered prior to mixing/precipitation.

The formed nanoparticles can be exposed to further processing techniques to remove the solvents or purify the nanoparticles (e.g., dialysis). For purposes of the aforementioned process, water miscible solvents include acetone, ethanol, methanol, and isopropyl alcohol; and partially water miscible organic solvents include acetonitrile, tetrahydrofuran, ethyl acetate, isopropyl alcohol, isopropyl acetate or dimethylformamide.

Another method that can be used to generate a particle described herein is a process termed “flash nanoprecipitation” as described by Johnson, B. K., et al, AlChE Journal (2003) 49:2264-2282 and U.S. 2004/0091546, each of which is incorporated herein by reference in its entirety. This process is capable of producing controlled size, polymer-stabilized and protected nanoparticles of hydrophobic organics at high loadings and yields. The flash nanoprecipitation technique is based on amphiphilic diblock copolymer arrested nucleation and growth of hydrophobic organics. Amphiphilic diblock copolymers dissolved in a suitable solvent can form micelles when the solvent quality for one block is decreased. In order to achieve such a solvent quality change, a tangential flow mixing cell (vortex mixer) is used. The vortex mixer consists of a confined volume chamber where one jet stream containing the diblock copolymer and nucleic acid agent dissolved in a water-miscible solvent is mixed at high velocity with another jet stream containing water, an anti-solvent for the nucleic acid agent and the hydrophobic block of the copolymer. The fast mixing and high energy dissipation involved in this process provide timescales that are shorter than the timescale for nucleation and growth of particles, which leads to the formation of nanoparticles with nucleic acid agent loading contents and size distributions not provided by other technologies. When forming the nanoparticles via flash nanoprecipitation, mixing occurs fast enough to allow high supersaturation levels of all components to be reached prior to the onset of aggregation. Therefore, the nucleic acid agent(s) and polymers precipitate simultaneously, and overcome the limitations of low active agent incorporations and aggregation found with the widely used techniques based on slow solvent exchange (e.g., dialysis). The flash nanoprecipitation process is insensitive to the chemical specificity of the components, making it a universal nanoparticle formation technique.

A particle described herein may also be prepared using a mixer technology, such as a static mixer or a micro-mixer (e.g., a split-recombine micro-mixer, a slit-interdigital micro-mixer, a star laminator interdigital micro-mixer, a superfocus interdigital micro-mixer, a liquid-liquid micro-mixer, or an impinging jet micro-mixer).

A split-recombine micromixer uses a mixing principle involving dividing the streams, folding/guiding over each other and recombining them per each mixing step, consisting of 8 to 12 such steps. Mixing finally occurs via diffusion within milliseconds, exclusive of residence time for the multi-step flow passage. Additionally, at higher-flow rates, turbulences add to this mixing effect, improving the total mixing quality further.

A slit interdigital micromixer combines the regular flow pattern created by multi-lamination with geometric focusing, which speeds up liquid mixing. Due to this double-step mixing, a slit mixer is amenable to a wide variety of processes.

A particle described herein may also be prepared using Microfluidics Reaction Technology (MRT). At the core of MRT is a continuous, impinging jet microreactor scalable to at least 50 lit/min. In the reactor, high-velocity liquid reactants are forced to interact inside a microliter scale volume. The reactants mix at the nanometer level as they are exposed to high shear stresses and turbulence. MRT provides precise control of the feed rate and the mixing location of the reactants. This ensures control of the nucleation and growth processes, resulting in uniform crystal growth and stabilization rates.

A particle described herein may also be prepared by emulsion. An exemplary emulsification method is disclosed in U.S. Pat. No. 5,407,609, which is incorporated herein by reference. This method involves dissolving or otherwise dispersing agents, liquids or solids, in a solvent containing dissolved wall-forming materials, dispersing the nucleic acid agent/polymer-solvent mixture into a processing medium to form an emulsion and transferring all of the emulsion immediately to a large volume of processing medium or other suitable extraction medium, to immediately extract the solvent from the microdroplets in the emulsion to form a microencapsulated product, such as microcapsules or microspheres. The most common method used for preparing polymer delivery vehicle formulations is the solvent emulsification-evaporation method. This method involves dissolving the polymer and drug in an organic solvent that is completely immiscible with water (for example, dichloromethane). The organic mixture is added to water containing a stabilizer, most often poly(vinyl alcohol) (PVA) and then typically sonicated.

After the particles are prepared, they may be fractionated by filtering, sieving, extrusion, or ultracentrifugation to recover particles within a specific size range. One sizing method involves extruding an aqueous suspension of the particles through a series of polycarbonate membranes having a selected uniform pore size; the pore size of the membrane will correspond roughly with the largest size of particles produced by extrusion through that membrane. See e.g., U.S. Pat. No. 4,737,323, incorporated herein by reference. Another method is serial ultracentrifugation at defined speeds (e.g., 8,000, 10,000, 12,000, 15,000, 20,000, 22,000, and 25,000 rpm) to isolate fractions of defined sizes. Another method is tangential flow filtration, wherein a solution containing the particles is pumped tangentially along the surface of a membrane. An applied pressure serves to force a portion of the fluid through the membrane to the filtrate side. Particles that are too large to pass through the membrane pores are retained on the upstream side. The retained components do not build up at the surface of the membrane as in normal flow filtration, but instead are swept along by the tangential flow. Tangential flow filtration may thus be used to remove excess surfactant present in the aqueous solution or to concentrate the solution via diafiltration.

An exemplary method of making a particle described herein includes combining, in polar solvent (e.g., DMF, DMSO, acetone, benzyl alcohol, dioxane, tetrahydrofuran, or acetonitrile) under conditions that allow formation of a particle, e.g., by precipitation, (a) nucleic acid agent-hydrophobic polymer conjugates, each nucleic acid agent-hydrophobic polymer conjugate comprising a nucleic acid agent, e.g., an siRNA moiety, covalently attached to a hydrophobic polymer, wherein the nucleic acid agent-hydrophobic polymer conjugates are associated with a cationic moiety, (b) a plurality of hydrophilic-hydrophobic polymers, e.g., PEG-PLGA, and (c) a plurality of hydrophobic polymers (not covalently attached to a nucleic acid agent) to thereby form a particle. The combining can be done in a polar solvent, for example, acetone, or in a mixed solvent system (e.g., a combination aqueous/organic solvent system such as acetonitrile and an aqueous buffer system). The method can also include: (i) a plurality of nucleic acid agents, each nucleic acid agent comprising a nucleic acid agent, e.g., an siRNA or other nucleic acid agent, coupled to a hydrophobic polymer and associated with a cationic moiety, in acetonitrile/TE buffer (e.g., 80/20 wt %); with (ii) a plurality of hydrophilic-hydrophobic polymers, e.g., PEG-PLGA, and a plurality of hydrophobic polymers (not coupled to a nucleic acid agent), in acetonitrile/TE buffer (e.g., 80/20 wt %).

Another exemplary method of making a particle described herein includes: a) contacting, e.g., in an aqueous solvent i) a first plurality of hydrophobic-hydrophilic polymers, e.g., PEG-PLGA, with ii) a first plurality of hydrophobic polymers, e.g., PLGA, each having a first reactive moiety, e.g., a sulfhydryl moiety; to form a water soluble intermediate particle (e.g., having a diameter of less than about 100 nm); b) contacting, e.g., in aqueous solvent the intermediate particle with a plurality of water soluble nucleic acid agent, e.g., siRNA moieties, each having a second reactive moiety, e.g., an SH moiety, under conditions which allow formation of an intermediate complex, e.g., an intermediate structure comprising hydrophilic-hydrophobic polymers and hydrophobic polymers coupled to the drug moiety; and c) contacting, e.g., in a non-aqueous solvent, e.g., DMF, DMSO, acetone, benzyl alcohol, dioxane, tetrahydrofuran, or acetonitrile, the intermediate complex with a second plurality of hydrophilic-hydrophobic polymers, e.g., PEG-PLGA, and a second plurality of hydrophobic polymers, e.g., PLGA, under conditions that allow the formation of a particle, thereby forming a particle (wherein the formed particle is larger than the intermediate particle).

Another exemplary method of making a particle described herein includes a) contacting, e.g., in acetonitrile/TE buffer (e.g., 80/20 wt %) i) a first plurality of hydrophilic-hydrophobic polymers, e.g., PEG-PLGA, with ii) a first plurality of hydrophobic polymers, e.g., PLGA, each having a first reactive moiety, e.g., a sulfhydryl moiety; to form an intermediate particle (e.g., having a diameter of less than about 100 nm), wherein, In some embodiments, the intermediate particle is functionally soluble in aqueous solution, e.g., by virtue of having sufficient hydrophilic portion such that it is soluble in aqueous solution; b) contacting the intermediate particle with a plurality of nucleic acid agents, e.g., siRNA or other nucleic acid agents, each having a second reactive moiety, e.g., an SH moiety, under conditions which allow formation of an intermediate complex, e.g., an intermediate structure comprising hydrophilic-hydrophobic polymers and hydrophobic polymers coupled to the nucleic acid agent and, c) contacting the intermediate complex with a second plurality of hydrophilic-hydrophobic polymers, e.g., PEG-PLGA, and a second plurality of hydrophobic polymers, e.g., PLGA, under conditions that allow the formation of a particle, thereby forming a particle (e.g., wherein the diameter of the particle is less than 150 nm). A plurality of cationic moieties can be covalently attached to the hydrophobic polymers from b.

Another exemplary method of making a particle described herein includes dissolving cationic-PLGA and nucleic acid-conjugated 5050-O-acetyl-PLGA into a solution. The resulting solution will be added to water to form a nanoparticle suspension. A lipid mixture, e.g., including DOTAP, cholesterol and DOPE-PEG₂ₖ would be added to the particle suspension under conditions to allow the lipid mixture to coat the particle.

Another exemplary method of making a particle described herein includes dissolving nucleic acid-conjugated 5050-O-acetyl-PLGA (Mw˜23.7 kDa) into a solution. The resulting solution will be added to water to form a nanoparticle suspension. A cationic polymer (e.g., polyhistidine, polylysine, polyarginine, polyethylene imine, and chitosan 60 wt. %) would be dissolved in acetone to form a 1% polymer solution and would be added to the particle suspension under conditions to allow the polymer mixture to coat the particle.

Another exemplary method of making a particle described herein includes forming a particle comprising a plurality of nucleic acid agent-polymer conjugates; contacting the particle with a plurality of cationic polyvalent polymers or lipids; and

contacting the product of b) with a plurality of polymers or lipids, wherein the a plurality of polymers or lipids substantially surround the product of b) forming the particle.

In some embodiments, the particle is further processed, for example, purified. Exemplary methods of purification include gel electrophoresis, capillary electrophoresis, gel permeation chromatography, dialysis, tangential flow filtration (e.g., using a 300 kDa filter), and size exclusion chromatography.

After purification of the particles, they may be sterile filtered (e.g., using a 0.22 micron filter) while in solution.

In certain embodiments, the particles are prepared to be substantially homogeneous in size within a selected size range. The particles are preferably in the range from 30 nm to 300 nm in their greatest diameter, (e.g., from about 30 nm to about 250 nm). The particles may be analyzed by techniques known in the art such as dynamic light scattering and/or electron microscopy, (e.g., transmission electron microscopy or scanning electron microscopy) to determine the size of the particles. In some embodiments, the particles may be analyzed using a Nanosight “Nanoparticle Tracking Analysis.” In some embodiments, the particles may be analyzed using a Paraytec ActiPix™ imaging system. The particles may also be tested for nucleic acid agent loading and/or the presence or absence of impurities (such as residual solvent).

Lyophilization

A particle described herein may be prepared for dry storage via lyophilization, commonly known as freeze-drying. Lyophilization is a process which extracts water from a solution to form a granular solid or powder. The process is carried out by freezing the solution and subsequently extracting any water or moisture by sublimation under vacuum. Advantages of lyophilization include maintenance of substance quality and minimization of therapeutic compound degradation. Lyophilization may be particularly useful for developing pharmaceutical drug products that are reconstituted and administered to a patient by injection, for example parenteral drug products. Alternatively, lyophilization is useful for developing oral drug products, especially fast melts or flash dissolve formulations.

Lyophilization may take place in the presence of a lyoprotectant, e.g., a lyoprotectant described herein. In some embodiments, the lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, aggregation of PEGylated particles during lyophilization may be reduced or minimized by the use of lyoprotectants comprising a cyclic oligosaccharide. Using suitable lyoprotectants provides lyophilized preparations that have extended shelf-lives.

The disclosure features liquid formulations and lyophilized preparations that comprise a cyclic oligosaccharide. In some embodiments, the liquid formulation or lyophilized preparation can comprise at least two carbohydrates, e.g., a cyclic oligosaccharide (e.g., a cyclodextran or derivative thereof) and a non-cyclic oligosaccharide (e.g., a non-cyclic oligosaccharide less than about 10, 8, 6, 4 monosaccharides in length, e.g., a monosaccharide or disaccharide). In some embodiments, the liquid formulations also comprise a reconstitution reagent.

Examples of suitable cyclic oligosaccharides, include, but are not limited to, α-cyclodextrins, β-cyclodextrins, such as 2-hydroxypropyl-β-cyclodextrins, β-cyclodextrin sulfobutylethers sodiums, γ-cyclodextrins, any derivative thereof, and any combination thereof.

In certain embodiments, the cyclic carbohydrate, e.g., cyclic oligosaccharide, may be included in a larger molecular structure such as a polymer. Suitable polymers are disclosed herein with respect to the polymer composition of the particle. In such embodiments, the cyclic oligosaccharide may be incorporated within a backbone of the polymer. See, e.g., U.S. Pat. No. 7,270,808 and U.S. Pat. No. 7,091,192, which disclose exemplary polymers that contain cyclodextrin moieties in the polymer backbone that can be used in accordance with the disclosure. The entire teachings of U.S. Pat. No. 7,270,808 and U.S. Pat. No. 7,091,192 are incorporated herein by reference. In some embodiments, the cyclic oligosaccharide may contain at least one oxidized occurrence.

A lyoprotectant comprising a cyclic oligosaccharide, may inhibit the rate of intermolecular aggregation of particles that include hydrophilic polymers such as PEG during their lyophilization and/or storage, and therefore, provide for extended shelf-life. Without wishing to be limited by theory, the mechanism for the cyclic oligosaccharide to prevent particle aggregation may be due to the cyclic oligosaccharide reducing or preventing the crystallization of the hydrophilic polymer such as PEG present in the particles during lyophilization. This may occur through the formation of an inclusion complex between a cyclic oligosaccharide and the hydrophilic polymer (e.g., PEG). Such a complex may be formed between a cyclodextrin and, for example, the chain of polyethylene glycol. The inside cavity of cyclodextrin is lipophilic, while the outside of the cyclodextrin is hydrophilic. These properties may allow for the formation of inclusion complexes with other components of the particles described herein. For the purpose of stabilizing the formulations during lyophilization, the poly(ethyleneglycol) chain may fit into the cavity of the cyclodextrins. An additional mechanism that may allow the cyclic oligosaccharide to reduced or minimized or prevent particle degradation relates to the formation of hydrogen bonds between the cyclic oligosaccharide and the hydrophilic polymer (PEG) during lyophilization. For example, hydrogen bonding between cyclodextrin and poly(ethyleneglycol) chains may prevent ordered polyethylene glycol structures such as crystals.

The cyclic oligosaccharide may be present in varying amounts in the formulations described herein. In certain embodiments, the cyclic oligosaccharide to liquid formulation ratio is in the range of from about 0.75:1 to about 3:1 by weight. In preferred embodiments, the cyclic oligosaccharide to total polymer ratio is in the range of from about 0.75:1 to about 3:1 by weight.

In preferred aspects, the formulation contains two or more carbohydrates, e.g., a cyclic oligosaccharide and a non-cyclic carbohydrate, e.g., a non-cyclic oligosaccharide, e.g., a non-cyclic oligosaccharide having 10, 8, 6, 4 or less monosaccharide units. As described herein, including a non-cyclic carbohydrate, e.g., a non-cyclic oligosaccharide, into a liquid formulation that is to be lyophilized can promote uptake of water by the resulting lyophilized preparation, and promote disintegration of the lyophilized preparation.

In preferred aspects, the lyophilized or liquid formulation comprises a cyclic oligosaccharide, such as an α-cyclodextrin, β-cyclodextrin, γ-cyclodextrin, any derivative thereof, and any combination thereof, and a non-cyclic oligosaccharide, e.g., a non-cyclic oligosaccharide described herein. In some preferred embodiments, the lyoprotectant comprises a cyclic oligosaccharide, such as an α-cyclodextrin, β-cyclodextrin, γ-cyclodextrin, any derivative thereof, and any combination thereof, and the non-cyclic oligosaccharide is a disaccharide, such as sucrose, lactose, maltose, trehalose, and derivatives thereof, and a monosaccharide, such as glucose. In one preferred embodiment, the lyoprotectant comprises a β-cyclodextrin or derivative thereof, such as 2-hydroxypropyl-β-cyclodextrin or β-cyclodextrin sulfobutylether; and the non-cyclic oligosaccharide is a disaccharide, such as sucrose. The β-cyclodextrin or derivative thereof and the non-cyclic oligosaccharide can be present in any suitable relative amounts. Preferably, the ratio of cyclic oligosaccharide to non-cyclic oligosaccharide (w/w) is from about 0.5:1.5 to about 1.5:0.5, and more preferably from 0.7:1.3 to 1.3:0.7. In some examples, the ratio of cyclic oligosaccharide to non-cyclic oligosaccharide (w/w) is 0.7:1.3, 1:0.7, 1:1, 1.3:1 or 1.3:0.7. When the liquid or lyophilized formulation comprises a particle described herein, the ratio of cyclic oligosaccharide plus non-cyclic oligosaccharide to polymer (w/w) is from about 1:1 to about 10:1, and preferably, from about 1.1 to about 3:1.

In certain embodiments, the lyophilized preparations may be reconstituted with a reconstitution reagent. In some embodiments, a suitable reconstitution reagent may be any physiologically acceptable liquid. Suitable reconstitution reagents include, but are not limited to, water, 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. To minimize the amount of surfactant in the reconstituted solution, only a sufficient amount of the vehicle may be provided to form a solution of the lyophilized preparation. Once dissolution of the lyophilized preparation is achieved, the resulting solution may be further diluted prior to injection with a suitable parenteral diluent. Such diluents are well known to those of ordinary skill in the art. These diluents are generally available in clinical facilities. Examples of typical diluents include, but are not limited to, Lactated Ringer's Injection, 5% Dextrose Injection, Sterile Water for Injection, and the like. However, because of its narrow pH range, pH 6.0 to 7.5, Lactated Ringer's Injection is most typical. Per 100 mL, lactated ringer's injection contains sodium chloride USP 0.6 g, sodium lactate 0.31 g, potassium chloride USP 0.03 g and calcium chloride₂H₂O USP 0.02 g. The osmolarity is 275 mOsmol/L, which is very close to isotonicity.

Accordingly, a liquid formulation can be a resuspended or rehydrated lyophilized preparation in a suitable reconstitution reagent. Suitable reconstitution reagents include physiologically acceptable carriers, e.g., a physiologically acceptable liquid as described herein. Preferably, resuspension or rehydration of the lyophilized preparations forms a solution or suspension of particles which have substantially the same properties (e.g., average particle diameter (Zave), size distribution (Dv₉₀, Dv₅₀), polydispersity, drug concentration) and morphology of the original particles in the liquid formulation of the disclosure before lyophilization, and further maintains the therapeutic agent to polymer ratio of the original liquid formulation before lyophilization. In certain embodiments, about 50% to about 100%, preferably about 80% to about 100%, of the particles in the resuspended or rehydrated lyophilized preparation maintain the size distribution and/or drug to polymer ratio of the particles in the original liquid formulation. Preferably, the Zave, Dv₉₀, and polydispersity of the particles in the formulation produced by resuspending a lyophilized preparation do not differ from the Zave, Dv₉₀, and polydispersity of the particles in the original solution or suspension prior to lyophilization by more than about 5%, more than about 10%, more than about 15%, more than about 20%, more than about 15%, more than about 30%, more than about 35%, more than about 40%, more than about 45%, or more than about 50%.

Preferably liquid formulations of this aspect contain particles, and are characterized by a higher polymer concentration (the concentration of polymer(s) that form the particle) than can be lyophilized and resuspended using either a lyoprotectant that comprises one or more carbohydrates (e.g., a cyclic oligosaccharide and/or a non-cyclic oligosaccharide). For example, the polymer concentration can be at least about 20 mg/mL, at least about 25 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 45 mg/mL, at least about 50 mg/mL, at least about 55 mg/mL, at least about 60 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, are at least about 100 mg/mL. For example, the liquid formulation can be a reconstituted lyophilized preparation.

### Methods of Storing Particles and Compositions

In another aspect, the disclosure features, a method of storing a conjugate, particle or composition, e.g., a pharmaceutical composition.

In an embodiment, methods of storing a conjugate, particle, or composition described herein include, e.g., the steps of,: (a) providing said nucleic acid-hydrophobic polymer conjugate, particle or composition disposed in a container; (b) storing said nucleic acid-hydrophobic polymer conjugate, particle or composition; and, optionally, (c) moving said container to a second location or removing all or an aliquot of said nucleic acid-hydrophobic polymer conjugate, particle or composition, from said container.

The nucleic acid-hydrophobic polymer conjugate, particle or composition can be in liquid, dry, lyophilized, or re-constituted (e.g., in a liquid as a solution or suspension) formulation or form. The nucleic acid-hydrophobic polymer conjugate, particle or composition can be stored in single, or multi-dose amounts, e.g., it can be stored in amounts sufficient for at least 2, 5, 10, or 100 dosages. In an embodiment, the method comprises dialyzing, diluting, concentrating, drying, lyophilizing, or packaging (e.g., disposing the material in a container) the nucleic acid-hydrophobic polymer conjugate, particle or composition. In an embodiment the method comprises combining the nucleic acid-hydrophobic polymer conjugate, particle or composition with another component, e.g., an excipient, lyoprotectant, or inert substance, e.g., an insert gas. In an embodiment the method comprises dividing a preparation of the nucleic acid-hydrophobic polymer conjugate, particle or composition into aliquouts, and optionally disposing a plurality of aliquouts in a plurality of containers. In embodiments nucleic acid-hydrophobic polymer conjugates, particle or composition, e.g., pharmaceutical composition, is stored for a period disclosed herein. In embodiments, after a period of storage, the stored nucleic acid-hydrophobic polymer conjugate, particle or composition, is evaluated, e.g., for aggregation, color, or other parameter.

In embodiments a nucleic acid-hydrophobic polymer conjugate, particle or composition described herein may be stored, e.g., in a container, for at least about 1 hour (e.g., at least about 2 hours, 4 hours, 8 hours, 12 hours, 24 hours, 2 days, 1 week, 1 month, 2 months, 3 months, 4 months, 5 months, 6 months, 1 year, 2 years or 3 years). Accordingly, described herein are containers including a nucleic acid-hydrophobic polymer conjugate, particle or composition described herein.

In embodiments, a nucleic acid-hydrophobic polymer conjugate, particle or composition may be stored under a variety of conditions, including ambient conditions, or other conditions described herein. In an embodiment a nucleic acid-hydrophobic polymer conjugate, particle or composition is stored at low temperature, e.g., at a temperature less than or equal to about 5° C. (e.g., less than or equal to about 4° C. or less than or equal to about 0° C.). A nucleic acid-hydrophobic polymer conjugate, particle or composition may also be frozen and stored at a temperature of less than about 0° C. (e.g., between −80° C. and −20° C.). A nucleic acid-hydrophobic polymer conjugate, particle or composition may also be stored under an inert atmosphere, e.g., an atmosphere containing an inert gas such as nitrogen or argon. Such an atmosphere may be substantially free of atmospheric oxygen and/or other reactive gases, and/or substantially free of moisture.

In some embodiments, a nucleic acid-hydrophobic polymer conjugate, particle or composition can be stored as a re-constituted formulation (e.g., in a liquid as a solution or suspension).

In an embodiment a nucleic acid-hydrophobic polymer conjugate, particle or composition described herein can be stored in a variety of containers, including a light-blocking container such as an amber vial. A container can be a vial, e.g., a sealed vial having a rubber or silicone enclosure (e.g., an enclosure made of polybutadiene or polyisoprene). A container can be substantially free of atmospheric oxygen and/or other reactive gases, and/or substantially free of moisture.

In another aspect, the disclosure features, a nucleic acid-hydrophobic polymer conjugate, particle or composition, disposed in a container, e.g., a container described herein, e.g., in an amount, form or formulation described herein.

### Methods of Evaluating Particles and Compositions

In another embodiment, the disclosure features a method of evaluating a particle for the presence or level of a contaminant, e.g., a contaminant which is a product of the conjugation of a nucleic acid to a hydrophobic polymer. For example, the particle can be evaluated for the presence of an unconjugated nucleic acid, unconjugated hydrophobic polymer, or other conjugation reaction side product, e.g. pyridinethiol.

In an embodiment, the particle can be evaluated for a second property, a physical property, e.g., average diameter. Further embodiments include the evaluation of a particle for a functional property, e.g., the ability to mediate knockdown of a target gene, e.g., as measured in an assay described herein. The method comprises:

providing a sample comprising one or a plurality of said particles, e.g., as a composition, e.g., a pharmaceutical composition;

evaluating, e.g., by a physical test, a property described herein, to provide a determined value for the property,

thereby evaluating a particle or preparation of particles.

In an embodiment the method comprises one or both of:
a) comparing the determined value with a reference or standard value, e.g., a range of values (e.g., value disclosed herein, or set by a regulatory agency, manufacturer, or compendia authority), or
b) responsive to said determination or comparison, classifying said particles.

In an embodiment, responsive to said determination or comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment, the determined value for a property is compared with a reference, and responsive to said comparison said particle or preparation of particles is classified, e.g., as suitable for use in human subjects, not suitable for use in human subjects, suitable for sale, meeting a release specification, or not meeting a release specification.

In some embodiments, the contaminant can be pyridinethiol. In some embodiments, the contaminant can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the contaminant can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the contaminant can be any of the reagents used in the conjugation reaction using “click chemistry”, e.g., copper catalyst or ruthenium catalyst.

In some embodiments, the contaminant is a solvent such as water, dimethylsulfoxide (DMSO), N,N-dimethylformamide (DMF), or acetonitrile.

In an embodiment a particle or preparation of particles is subjected to a measurement to determine whether an impurity or residual solvent is present (e.g., via gas chromatography (GC)), to determine relative amounts of one or more components (e.g., via high performance liquid chromatography (HPLC)), to measure particle size (e.g., via dynamic light scattering and/or scanning electron microscopy), or determine the presence or absence of surface components.

In an embodiment a particle or preparation of particles is evaluated for the average diameter of the particles in the composition. In an embodiment experiments including physical measurements are performed to determine average value. The average diameter of the composition can then be compared with a reference value. In an embodiment the average diameter for the particles is about 50 nm to about 500 nm (e.g., from about 50 nm to about 200 nm). A composition of a plurality of particles particle may have a median particle size (Dv50 (particle size below which 50% of the volume of particles exists) of about 50 nm to about 500 nm (e.g., about 75 nm to about 220 nm)) from about 50 nm to about 220 nm (e.g., from about 75 nm to about 200 nm). A composition of a plurality of particles may have a Dv90 (particle size below which 90% of the volume of particles exists) of about 50 nm to about 500 nm (e.g., about 75 nm to about 220 nm). In some embodiments, a composition of a plurality of particles has a Dv90 of less than about 150 nm. A composition of a plurality of particles may have a particle PDI of less than 0.5, less than 0.4, less than 0.3, less than 0.2, or less than 0.1.

In an embodiment a particle or preparation of particles is subjected to dynamic light scattering, e.g., to determine size or diameter. Particles may be illuminated with a laser, and the intensity of the scattered light fluctuates at a rate that is dependent upon the size of the particles as smaller particles are “kicked” further by the solvent molecules and move more rapidly. Analysis of these intensity fluctuations yields the velocity of the Brownian motion and hence the particle size using the Stokes-Einstein relationship. The diameter that is measured in dynamic light scattering is called the hydrodynamic diameter and refers to how a particle diffuses within a fluid. The diameter obtained by this technique is that of a sphere that has the same translational diffusion coefficient as the particle being measured.

In an embodiment a particle or preparation of particles is evaluated using cryo scanning electron microscopy (Cryo-SEM), e.g., to determine structure or composition. SEM is a type of electron microscopy in which the sample surface is imaged by scanning it with a high-energy beam of electrons in a raster scan pattern. The electrons interact with the atoms that make up the sample producing signals that contain information about the sample's surface topography, composition and other properties such as electrical conductivity. For Cryo-SEM, the SEM is equipped with a cold stage for cryo-microscopy. Cryofixation may be used and low-temperature scanning electron microscopy performed on the cryogenically fixed specimens. Cryo-fixed specimens may be cryo-fractured under vacuum in a special apparatus to reveal internal structure, sputter coated and transferred onto the SEM cryo-stage while still frozen.

In an embodiment a particle or preparation of particles is evaluated using transmission electron microscopy (TEM), e.g., to determine structure or composition. In this technique, a beam of electrons is transmitted through an ultra thin specimen, interacting with the specimen as it passes through. An image is formed from the interaction of the electrons transmitted through the specimen; the image is magnified and focused onto an imaging device, such as a fluorescent screen, on a layer of photographic film, or to be detected by a sensor such as a charge-coupled device (CCD) camera.

In an embodiment a particle or preparation of particles is evaluated for a surface zeta potential. In an embodiment experiments including physical measurements are performed to determine average value a surface zeta potential. The surface zeta potential can then be compared with a reference value. In an embodiment the surface zeta potential is between about −20 mV to about 50 mV, when measured in water. Zeta potential is a measurement of surface potential of a particle. In some embodiments, a particle may have a surface zeta potential, when measured in water, ranging between about −20 mV to about 20 mV, about −10 mV to about 10 mV, or neutral.

In an embodiment a particle or preparation of particles is evaluated for the effective amount of nucleic acid agent (e.g., an siRNA) it contains. In embodiment particles are administered, for example, in an in vivo model system, (e.g., a mouse model such as any of those described herein), and the level of effect (e.g., knock-down) observed. In embodiments the level is compared with a reference standard.

In an embodiment a particle or preparation of particles is evaluated for the presence of nucleic acid agent on its surface. For example, an intercalating agent such as RIBOGREEN, or HPLC, can be used to determine the presence or amount of a double stranded nucleic acid agent on the surface of the particle (e.g., the presence or amount of siRNA).

In an embodiment a particle or preparation of particles is evaluated for the amount of nucleic acid agent, e.g., siRNA, inside, as opposed to exposed at the surface, of the particle. In embodiments the level is compared with a reference standard. In embodiments at least 30, 40, 50, 60, 70, 80, or 90% of the nucleic acid agent, e.g., siRNA, by number or weight, in a particle is inside the particle.

In an embodiment a particle or preparation of particles is evaluated using an assay that provides information about the structure or function of the nucleic acid agent (e.g., a digestion assay). For example, the particle can be evaluated in an experiment that evaluates the ability of the nucleic acid agent to modulate expression of a target (e.g., knockdown). The particle can also be evaluated for its ability to treat a disorder, e.g, modulate tumor growth. In some embodiments, the evaluation is in an in vitro or in vivo assay (e.g., a xenograph model). The evaluation can be compared to a standard, and optionally, responsive to said standard, the particle is classified.

In an embodiment a particle or preparation of particles is evaluated for the ability to deliver a nucleic acid agent, e.g., an siRNA, that knocks down a target gene, in vivo, e.g., in an experimental animal, e.g., a mouse. The activity of the composition can be compared to that of an equal amount of free nucleic acid agent. In some embodiments the target gene is GFP the GFP is expressed in HeLA cells. E.g., the assay can use the anti-GFP siRNA, the GFP plasmid, the HeLA-GFP cells, the mice, and the GFP expression assays described in Bertrand et al., 2002, BBRC 296:1000-1004, hereby incorporated by reference. Other exemplary cells for evaluating nucleic acid-hydrophobic polymer conjugates, particles, and compositions include MDA-MB-435 and MDA-MB-468 GFP cells.

In an embodiment a particle or preparation of particles is evaluated for the ability to deliver a nucleic acid agent, e.g., an siRNA, that knocks down a target gene in vitro, e.g., in cultured cells. The activity of the composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, and the GFP expression assay described in Bertrand et al., 2002, BBRC 296:1000-1004, hereby incorporated by reference. Other exemplary cells for evaluating particles and compositions described herein include MDA-MB-435 and MDA-MB-468 GFP cells.

In an embodiment a particle or preparation of particles is evaluated for the ability to deliver a nucleic acid agent, e.g., an siRNA, that knocks down a target gene in vitro, e.g., in cultured cells, after incubation in serum or a cell lysate. The activity of the treated composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, the GFP expression assay, and, in the case of an assay that uses a cell lysate, the HeLa cell lysate, described in Bertrand et al., 2002, BBRC 296:1000-1004, hereby incorporated by reference. Alternatively, the mouse expression system described in Hu-Lieskovan et al., 2005, Cancer Res. 65: 8984-8992, hereby incorporated by reference, can be used to evaluate the performance of a composition. The target gene and constructs of Hu-Lieskovan et al., or other target genes and constructs can be used with the mouse system described in Hu-lieskovan et al. Other exemplary cells for evaluating particles and compositions described herein include MDA-MB-435 and MDA-MB-468 GFP cells.

In an embodiment a particle or preparation of particles is evaluated for the ability to protect a nucleic acid agent from a degradant such as an RNase (e.g., RNase A). In some embodiments, a composition described herein can confer protection on a nucleic acid agent such as an siRNA relative to untreated nucleic acid agent (e.g., free siRNA). The evaluation can include an assay where the composition and/or free nucleic acid agent is incubated with a degradant such as an RNase, and, e.g., wherein the composition and free nucleic acid are evaluated over various time points, e.g., using gel chromatography.

In an embodiment a particle or preparation of particles is evaluated for the level of intact nucleic acid agent (e.g., an siRNA) it contains. In embodiment the intactness can be determined by presence of a physical property, e.g., molecular weight, or by functionality for example, in an in vivo model system, (e.g., a mouse model such as any one of those described herein). In embodiments the level is compared with a reference standard. In embodiments at least 30, 40, 50, 60, 70, 80, or 90% of the nucleic acid agent, e.g., siRNA, by number or weight, in a particle may be intact.

In an embodiment a particle or preparation of particles is evaluated for its tendency to aggregate. E.g., aggregation can be measured in a preselected medium, e.g., 50/50 mouse/human serum. In embodiment, when incubated 50/50 mouse human serum, the particles exhibit little or no aggregation. E.g., less than 30, 20, or 10%, by number or weight, of the particles will aggregate. In embodiments the level is compared with a reference standard.

In an embodiment a particle or preparation of particles is evaluated for stability, e.g., stability at a preselected condition, e.g., at 25° C.±2° C./60% relative humidity±5% relative humidity, e.g., in an open, or closed, container. In embodiments, when stored at 25° C.±2° C./60% relative humidity±5% relative humidity in an open, or closed, container, for 20, 30, 40, 50 or 60 days, the particle retains at least 30, 40, 50, 60, 70, 80, 90, or 95% of its activity, e.g., as determined in an in vivo model system, (e.g., a mouse model such as one described herein). In embodiments the level of retained activity is compared with a reference standard.

In an embodiment a particle or preparation of particles is evaluated its ability to reduce protein and or mRNA, e.g., at a preselected dosage. E.g., particles can be evaluated by administration as a single dose of 1 or 3 mg/kg in an in vivo model system, (e.g., a mouse model such one of those described herein). A particle described herein may result in at least 20, 30, 40, 50, or 60% reduction in protein and or mRNA knockdown. In embodiments the level is compared with a reference standard.

In an embodiment a particle or preparation of particles is evaluated its ability to reduce protein and or mRNA, of a target gene, e.g., at a preselected dosage. E.g., particles can be evaluated by administration as a single dose of 1 or 3 mg/kg in an in vivo model system, (e.g., a mouse model such as any of those described herein). A particle described herein may result in at least 20, 30, 40, 50, or 60% reduction in protein and or mRNA knockdown. In embodiments the level is compared with a reference standard.

In an embodiment a particle or preparation of particles is evaluated for reduction of protein and or mRNA, of an off-target gene, e.g., at a preselected dosage. E.g., particles can be evaluated by administration, e.g., as a single dose of 1 or 3 mg/kg in an in vivo model system, (e.g., a mouse model such as any of those described herein). A particle or preparation described herein may result in less than 20, 10, 5%, or no knockdown, as measured by protein or mRNA, when administered (e.g., as a single dose of 1 or 3 mg/kg) in an in vivo model system, (e.g., a mouse model such as any of those described herein).

In an embodiment a particle or preparation of particles is evaluated for the ability to cleave mRNA.

In an embodiment a particle or preparation of particles is evaluated for the ability to induce cytokines. A particle or preparation described herein may result in less than 2, 5, or 10 fold cytokine induction, when administered (e.g., as a single dose of 1 or 3 mg/kg) in an in vivo model system, (e.g., a mouse model such as any of those described herein). E.g., the administration results in less than 2, 5, or 10 fold induction of one, or more, e.g., two, three, four, five, six, or seven, or all, of: tumor necrosis factor-alpha, interleukin-1alpha, interleukin-1beta, interleukin-6, interleukin-10, interleukin-12, keratinocyte-derived cytokine and interferon-gamma.

In an embodiment a particle or preparation of particles is evaluated for the ability to increase in alanine aminotransferase (ALT) and or aspartate aminotransferase (AST), when administered (e.g., as a single dose of 1 or 3 mg/kg) in an in vivo model system, (e.g., a mouse model such as any of those described herein). In an embodiment a particle or preparation results in less than 2, 5, or 10 fold increase.

In an embodiment a particle or preparation of particles is evaluated for the ability to alter blood count. In an embodiment a particle or preparation results in no changes in blood count, e.g., no change 48 hours after 2 doses of 3 mg/kg in an in vivo model system, (e.g., a mouse model such as any of those described herein).

A particle described herein may be subjected to a number of analytical methods. For example, a particle described herein may be subjected to a measurement to determine whether an impurity or residual solvent is present (e.g., via gas chromatography (GC)), to determine relative amounts of one or more components (e.g., via high performance liquid chromatography (HPLC)), to measure particle size (e.g., via dynamic light scattering and/or scanning electron microscopy), or determine the presence or absence of surface components.

Compositions disclosed herein can be evaluated, for example, for the ability to deliver a nucleic acid agent, e.g., an siRNA, that knocks down a target gene, in vivo, e.g., in an experimental animal, e.g., a mouse. The activity of the composition can be compared to that of an equal amount of free nucleic acid agent. In some embodiments the target gene is GFP (e.g., an EGFP) the GFP is expressed in HeLA cells. E.g., the assay can use the anti-GFP siRNA, the GFP plasmid, the HeLA-GFP cells, the mice, and the GFP expression assays described in Bertrand et al., 2002, BBRC 296:1000-1004, hereby incorporated by reference. Other exemplary cells for evaluating particles and compositions described herein include MDA-MB-435 and M4A4 GFP cells.

Compositions disclosed herein can be evaluated for the ability to deliver a nucleic acid agent, e.g., an siRNA, that knocks down a target gene in vitro, e.g., in cultured cells. The activity of the composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, and the GFP expression assay described in Bertrand et al., 2002, BBRC 296:1000-1004, hereby incorporated by reference. Other exemplary cells for evaluating particles and compositions described herein include MDA-MB-435 and M4A4 GFP cells.

Compositions disclosed herein can be evaluated for the ability to deliver a nucleic acid agent, e.g., an siRNA, that knocks down a target gene in vitro, e.g., in cultured cells, after incubation in serum or a cell lysate. The activity of the treated composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, the GFP expression assay, and, in the case of an assay that uses a cell lysate, the HeLa cell lysate, described in Bertrand et al., 2002, BBRC 296:1000-1004, hereby incorporated by reference. Alternatively, the mouse expression system described in Hu-Lieskovan et al., 2005, Cancer Res. 65: 8984-8992, hereby incorporated by reference, can be used to evaluate the performance of a composition. The target gene and constructs of Hu-Lieskovan et al., or other target genes and constructs can be used with the mouse system described in Hu-lieskovan et al. Other exemplary cells for evaluating particles and compositions described herein include MDA-MB-435 and M4A4 GFP cells.

Compositions disclosed herein can be evaluated for the ability to protect a nucleic acid agent from a degradant such as an RNase (e.g., RNase A). In some embodiments, a composition described herein can confer protection on a nucleic acid agent such as an siRNA relative to untreated nucleic acid agent (e.g., free siRNA). The evaluation can include an assay where the composition and/or free nucleic acid agent is incubated with a degradant such as an RNase, and wherein the composition and free nucleic acid are evaluated over various time points, e.g., using gel chromatography.

### Methods of Evaluating a Preparation of a Nucleic Acid-Hydrophobic Polymer Conjugate

In another aspect, the disclosure features a method of evaluating a preparation of a nucleic acid-hydrophobic polymer conjugate for the presence or level of a contaminant, e.g., a contaminant which is a product of the conjugation of a nucleic acid to a hydrophobic polymer. For example, the particle can be evaluated for the presence of an unconjugated nucleic acid, unconjugated hydrophobic polymer, or other conjugation reaction side product, e.g. pyridinethiol.

The method comprises:

providing a the preparation;

evaluating, e.g., by a physical test, a property described herein, to provide a determined value for the property,

thereby evaluating the preparations.

In an embodiment the method comprises one or both of:
a) comparing the determined value with a reference or standard value, e.g., a range of values (e.g., value disclosed herein, or set by a regulatory agency, manufacturer, or compendia authority), or
b) responsive to said determination or comparison, classifying said preparation.

In some embodiments, the contaminant can be pyridinethiol.

In some embodiments, the contaminant can be an unconjugated activated nucleic acid, which can include both quenched and/or reduced reactive moieties on the nucleic acid. In some embodiments, the reactive moiety can be on either the 3′ or 5′ hydroxyl group of the nucleic acid. In some embodiments, the unconjugated nucleic acid, can include nucleic acids that do not have a reactive moiety, e.g. from the incomplete reaction that converts an unactivated nucleic acid to an activated nucleic acid. In some embodiments, the unconjugated nucleic acid can be a dimer of nucleic acids formed by the conjugation of two activated nucleic acids.

In some embodiments, the contaminant can be an unconjugated hydrophobic polymer, e.g. an activated hydrophobic polymer, which can be either quenched or reduced or still active. In some embodiments, the activated hydrophobic polymer can have a reactive moiety that is still active such as an azide moiety, an activated carboxylic acid moiety, or a disulfanylpyridine moiety. In some embodiments, the activated hydrophobic polymer can be a pyridinyl-SS-activated polymer, a maleimide-activated polymer, an acrylate-activated polymer, and aldehyde-activated polymer, or an azide-activated polymer. In some embodiments, the quenched activated hydrophobic polymer can include a thiol moiety, an amine moiety, or a carboxylic acid moiety. In some embodiments, the unconjugated hydrophobic polymer can include polymers that do not have an activated/reactive moiety, e.g. from the incomplete reaction that converts an unactivated hydrophobic polymer to an activated hydrophobic polymer. In some embodiments, the hydrophobic polymer can be a dimer formed by the reaction between two activated hydrophobic polymers. In some embodiments, the unconjugated hydrophobic polymer can have a molecular weight of about 10 kDa to about 60 kDa. In some embodiments, the hydrophobic polymer can comprise PLGA.

In some embodiments, the contaminant can be any of the reagents used in the conjugation reaction using “click chemistry”, e.g., copper catalyst or ruthenium catalyst.

In some embodiments, the contaminant is a solvent such as water, dimethylsulfoxide (DMSO), N,N-dimethylformamide (DMF), or acetonitrile.

In some embodiments, one or more of the components, e.g., contaminants, described herein can be present in a preparation comprising a nucleic acid-hydrophobic polymer conjugate in an amount that is less than 20, 15, 10, 5, 1, 0.1, 0.01% by weight of the component.

### Pharmaceutical Compositions

Provided herein is a composition, e.g., a pharmaceutical composition, comprising a plurality of particles described herein, e.g., a particle comprising a nucleic acid-hydrophobic polymer conjugate, and a pharmaceutically acceptable carrier or adjuvant.

In some embodiments, a pharmaceutical composition may include a pharmaceutically acceptable salt of a compound described herein, e.g., a nucleic acid-hydrophobic polymer conjugate. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This disclosure also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a nucleic acid-hydrophobic polymer conjugate, particle or composition, which may be any liquid solution compatible with the nucleic acid-hydrophobic polymer conjugate, particle or composition, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, D5W and mixtures thereof.

A composition described herein may also include another component, such as an antioxidant, antibacterial, buffer, bulking agent, chelating agent, an inert gas, a tonicity agent and/or a viscosity agent.

In one embodiment, the nucleic acid-hydrophobic polymer conjugate, particle or composition is provided in lyophilized form and is reconstituted prior to administration to a subject. The lyophilized nucleic acid-hydrophobic polymer conjugate, particle or composition can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the particle and active from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxamers, polysorbates, polyoxyethylene esters, lecithins, saccharides, oligosaccharides, polysaccharides, carbohydrates, cyclodextrins (e.g. 2-hydroxypropyl-β-cyclodextrin) and polyols (e.g., trehalose, mannitol, sorbitol, lactose, sucrose, glucose and dextran), salts and crown ethers.

In some embodiments, the lyophilized nucleic acid-hydrophobic polymer conjugate, particle or composition is reconstituted with water, 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution of the nucleic acid-hydrophobic polymer conjugate, particle or composition. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the disclosure to package the subject nucleic acid-hydrophobic polymer conjugate, particle or composition with a third vial containing sufficient parenteral diluent to prepare the final concentration for administration. A typical diluent is Lactated Ringer's Injection.

The final dilution of the reconstituted nucleic acid-hydrophobic polymer conjugate, particle or composition may be carried out with other preparations having similar utility, for example, 5% dextrose injection, lactated ringer's and dextrose injection, sterile water for injection, and the like. However, because of its narrow pH range, pH 6.0 to 7.5, lactated ringer's injection is most typical. Per 100 mL, Lactated Ringer's Injection contains sodium chloride USP 0.6 g, Sodium Lactate 0.31 g, potassium chloride USP 0.03 g and calcium chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of nucleic acid agent which can be combined with a pharmaceutically acceptable carrier to produce a single dosage form will vary depending upon the host being treated, the particular mode of administration. The amount of nucleic acid agent which can be combined with a pharmaceutically acceptable carrier to produce a single dosage form will generally be that amount of the compound which produces a therapeutic effect.

### Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional, intraocular, or intracranial injection), topically, mucosally (e.g., rectally or vaginally), nasally, buccally, ophthalmically, via inhalation spray (e.g., delivered via nebulization, propellant or a dry powder device) or via an implanted reservoir.

Pharmaceutical compositions suitable for parenteral administration comprise one or more nucleic acid-hydrophobic polymer conjugate(s), particle(s) or composition(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a nucleic acid agent, it is desirable to slow the absorption of the agent from subcutaneous or intramuscular injection. This may be accomplished by the use of a liquid suspension of crystalline or amorphous material having poor water solubility. The rate of absorption of the nucleic acid-hydrophobic polymer conjugate, particle or composition then depends upon its rate of dissolution which, in turn, may depend upon crystal size and crystalline form. Alternatively, delayed absorption of a parenterally administered drug form is accomplished by dissolving or suspending the nucleic acid-hydrophobic polymer conjugate, particle or composition in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A composition may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the polymer-agent nucleic acid-hydrophobic polymer conjugate, particle or composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the nucleic acid-hydrophobic polymer conjugate, particle or composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more nucleic acid-hydrophobic polymer conjugate, particle or composition described herein with one or more suitable non-irritating excipients which is solid at room temperature, but liquid at body temperature. The composition will therefore melt in the rectum or vaginal cavity and release the nucleic acid-hydrophobic polymer conjugate, particle or composition. Such materials include, for example, cocoa butter, polyethylene glycol, a suppository wax or a salicylate. Compositions of the disclosure which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the disclosure. An ocular tissue (e.g., a deep cortical region, a supranuclear region, or an aqueous humor region of an eye) may be contacted with the ophthalmic formulation, which is allowed to distribute into the lens. Any suitable method(s) of administration or application of the ophthalmic formulations of the disclosure (e.g., topical, injection, parenteral, airborne, etc.) may be employed. For example, the contacting may occur via topical administration or via injection.

### Dosages and Dosage Regimens

The nucleic acid-hydrophobic polymer conjugates, particles, and compositions can be formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular subject, composition, and mode of administration, without being toxic to the subject.

In one embodiment, the nucleic acid-hydrophobic polymer conjugate, particle or composition is administered to a subject at a dosage of, e.g., about 0.001 to 300 mg/m², about 0.002 to 200 mg/m², about 0.005 to 100 mg/m², about 0.01 to 100 mg/m², about 0.1 to 100 mg/m², about 5 to 275 mg/m², about 10 to 250 mg/m², e.g., about 0.001, 0.002, 0.005, 0.01, 0.05, 0.1, 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m². Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the nucleic acid-hydrophobic polymer conjugate, particle or composition is administered as a bolus infusion or intravenous push, e.g., over a period of 15 minutes, 10 minutes, 5 minutes or less. In one embodiment, the nucleic acid-hydrophobic polymer conjugate, particle or composition is administered in an amount such the desired dose of the agent is administered. Preferably the dose of the nucleic acid-hydrophobic polymer conjugate, particle or composition is a dose described herein.

In one embodiment, the subject receives 1, 2, 3, up to 10, up to 12, up to 15 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The nucleic acid-hydrophobic polymer conjugate, particle, or composition can be administered as a first line therapy, e.g., alone or in combination with an additional agent or agents. In other embodiments, a nucleic acid-hydrophobic polymer conjugate, particle or composition is administered after a subject has developed resistance to, has failed to respond to or has relapsed after a first line therapy. The nucleic acid-hydrophobic polymer conjugate, particle or composition may be administered in combination with a second agent. Preferably, the nucleic acid-hydrophobic polymer conjugate, particle or composition is administered in combination with a second agent described herein. The second agent may be the same or different as the nucleic acid agent in the particle.

### Kits

A nucleic acid-hydrophobic polymer conjugate, particle or composition described herein may be provided in a kit. The kit includes a nucleic acid-hydrophobic polymer conjugate, particle or composition described herein and, optionally, a container, a pharmaceutically acceptable carrier and/or informational material. The informational material can be descriptive, instructional, marketing or other material that relates to the methods described herein and/or the use of the particles for the methods described herein.

The informational material of the kits is not limited in its form. In one embodiment, the informational material can include information about production of the nucleic acid-hydrophobic polymer conjugate, particle or composition, physical properties of the nucleic acid-hydrophobic polymer conjugate, particle or composition, concentration, date of expiration, batch or production site information, and so forth. In one embodiment, the informational material relates to methods for administering the nucleic acid-hydrophobic polymer conjugate, particle or composition.

In one embodiment, the informational material can include instructions to administer a nucleic acid-hydrophobic polymer conjugate, particle or composition described herein in a suitable manner to perform the methods described herein, e.g., in a suitable dose, dosage form, or mode of administration (e.g., a dose, dosage form, or mode of administration described herein). In another embodiment, the informational material can include instructions to administer a nucleic acid-hydrophobic polymer conjugate, particle or composition described herein to a suitable subject, e.g., a human, e.g., a human having or at risk for a disorder described herein. In another embodiment, the informational material can include instructions to reconstitute a nucleic acid-hydrophobic polymer conjugate or particle described herein into a pharmaceutically acceptable composition.

In one embodiment, the kit includes instructions to use the nucleic acid-hydrophobic polymer conjugate, particle or composition, such as for treatment of a subject. The instructions can include methods for reconstituting or diluting the nucleic acid-hydrophobic polymer conjugate, particle or composition for use with a particular subject or in combination with a particular chemotherapeutic agent. The instructions can also include methods for reconstituting or diluting the nucleic acid-hydrophobic polymer conjugate composition for use with a particular means of administration, such as by intravenous infusion.

In another embodiment, the kit includes instructions for treating a subject with a particular indication. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a particle described herein and/or its use in the methods described herein. The informational material can also be provided in any combination of formats.

In addition to a nucleic acid-hydrophobic polymer conjugate, particle or composition described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a particle described herein. In such embodiments, the kit can include instructions for admixing a nucleic acid-hydrophobic polymer conjugate, particle or composition described herein and the other ingredients, or for using a nucleic acid-hydrophobic polymer conjugate, particle or composition described herein together with the other ingredients.

In another embodiment, the kit includes a second therapeutic agent. In one embodiment, the second agent is in lyophilized or in liquid form. In one embodiment, the nucleic acid-hydrophobic polymer conjugate, particle or composition and the second therapeutic agent are in separate containers, and in another embodiment, the nucleic acid-hydrophobic polymer conjugate, particle or composition and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under nitrogen or another inert gas such as argon). In some embodiments, a component of the kit is stored under anhydrous conditions (e.g., with a desiccant). In some embodiments, a component of the kit is stored in a light blocking container such as an amber vial.

A nucleic acid-hydrophobic polymer conjugate, particle or composition described herein can be provided in any form, e.g., liquid, frozen, dried or lyophilized form. It is preferred that a nucleic acid-hydrophobic polymer conjugate, particle or composition described herein be substantially pure and/or sterile. In some embodiments, the nucleic acid-hydrophobic polymer conjugate, particle or composition is sterile. When a nucleic acid-hydrophobic polymer conjugate, particle or composition described herein is provided in a liquid solution, the liquid solution preferably is an aqueous solution, with a sterile aqueous solution being preferred. In one embodiment, the nucleic acid-hydrophobic polymer conjugate, particle or composition is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a nucleic acid-hydrophobic polymer conjugate, particle or composition described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a nucleic acid-hydrophobic polymer conjugate, particle or composition described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Methods of Using Particles and Compositions

The nucleic acid-hydrophobic polymer conjugates, particles and compositions described herein can be administered to cells in culture, e.g. in vitro or ex vivo, or to a subject, e.g., in vivo, to treat or prevent a variety of diseases or disorders (e.g., cancer (for example solid tumors), autoimmune disorders, cardiovascular disorders, inflammatory disorders, metabolic disorders, infectious diseases, etc.).

Thus, in another aspect, the disclosure features, a method of treating or preventing a disease or disorder in a subject wherein the disease or disorder is cancer (for example a solid tumor), an autoimmune disorder, a cardiovascular disorder, inflammatory disorder, a metabolic disorder, or an infectious disease. The method comprises administering an effective amount of a nucleic acid-hydrophobic polymer conjugate, particle, or composition described herein to thereby treat the disease or disorder. In an embodiment the nucleic acid-hydrophobic polymer conjugates, particles and compositions can be used as part of a first line, second line, or adjunct therapy, and can also be used alone or in combination with one or more additional treatment regimes.

In an embodiment nucleic acid-hydrophobic polymer conjugates, particles, or compositions disclosed herein can be used to treat or prevent a wide variety of diseases or disorders and can be used to deliver nucleic acid agents, for example, to a subject in need thereof, for example, antisense or siRNA; to treat diseases and disorders described herein such as cancer, inflammatory or autoimmune disease, or cardiovascular disease, including those listed in the following tables A, B, or C. In embodiments the nucleic acid-hydrophobic polymer conjugates, particles and compositions can be used as part of a first line, second line, or adjunct therapy, and can also be used alone or in combination with one or more additional treatment regimes.

### Cancer

In another aspect, the disclosure features, a method of treating or preventing a disease or disorder in a subject wherein the disease or disorder is cancer (for example a solid tumor). The method comprises administering an effective amount of a nucleic acid-hydrophobic polymer conjugate, particle, or composition described herein to thereby treat the disease or disorder. In an embodiment the nucleic acid-hydrophobic polymer conjugates, particles and compositions can be used as part of a first line, second line, or adjunct therapy, and can also be used alone or in combination with one or more additional treatment regimes.

In embodiments the disclosed nucleic acid-hydrophobic polymer conjugates, particles and compositions are used to treat or prevent proliferative disorders, e.g., treating a tumor and metastases thereof wherein the tumor or metastases thereof is a cancer described herein. In some embodiments, wherein the agent is a diagnostic agent, the nucleic acid-hydrophobic polymer conjugates, particles and compositions described herein can be used to evaluate or diagnose a cancer.

In embodiments, the proliferative disorder is a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. In embodiments the method comprises evaluating or treating soft tissue tumors such as those of the tendons, muscles or fat, and liquid tumors.

In embodiment the cancer is any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and pregnancy;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sézary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

The nucleic acid-hydrophobic polymer conjugates, compounds or compositions described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In one embodiment, a method is provided for a combination treatment of a cancer, such as by treatment with a nucleic acid-hydrophobic polymer conjugate, compound or composition and a second therapeutic agent. Various combinations are described herein. The combination can reduce the development of tumors, reduces tumor burden, or produce tumor regression in a mammalian host.

In an embodiment, a nucleic acid-hydrophobic polymer conjugate, particle or composition, e.g., containing an siRNA that targets a gene listed in Table 3, is administered, e.g, to treat or prevent, an associated disease listed in Table 3.

TABLE 3
The nucleic acid, e.g. nucleic acid agent, e.g., an siRNA, can target a gene
listed in the table, for example, to treat or prevent the associated disease.
Cancer

| | Disease Associated with siRNA knock |
|---|---|
| Gene | down of gene |
| ICAM-1 | Angiogenesis (associated with cancer: |
| | breast, lung, head and neck, brain, abdominal, |
| | colon, colorectal, esophagus, gastrointestinal, |
| | glioma, liver, tongue, neuroblastoma, |
| | osteosarcoma, ovarian, pancreatic, prostate, |
| | retinoblastoma, Wilm's tumor, multiple myeloma, |
| | skin, lymphoma, blood, tumor metastasis, multiple |
| | myeloma) |
| NPRA | Melanoma, lung, ovarian |
| Akt & p85alpha | Colorectal |
| IL-1, TNFalpha, Fas, FasL | Liver |
| RAS, MYC, FOS, JUN, ERG-2, | Cancer |
| VEGF, FGF, Hcg | |
| KLF5 | Angiogenesis |
| Beta-TrCRP1, Beta-TrCP2, RSK1, | Cancer |
| RSK2 | |
| Notch1 | Cancer |
| HER2 | Breast |
| CD24 | Colorectal |
| ILK | Cancer |
| Nrf2 | Lung |
| Agtr11, Apelin, Stabilin 1, Stabilin | Angiogenesis |
| 2, TNFaip811, TNFaip8, FGD5 | |
| STAT3 | Cancer |
| HIF-1alpha | Cancer |
| STAT5 | Cancer |
| EGR, XIAP | Cancer |
| Akt2 | Cancer |
| TRIM24 | Breast, retinal, prostate, colon, acute |
| | lymphoblastic leukemia |
| Src-1, Src-2, Src-3, AIB1 | Cancer |
| ANT2 | Cancer |
| EGFR | Breast, lung, colorectal, prostate, brain, |
| | esophageal, stomach, bladder, pancreatic, cervical, |
| | head and neck, kidney, endometrial, ovarian, |
| | meningioma, melanoma, lymphoma, glioblastoma |
| CACNA1E | Breast, lung, liver, colon, prostate, renal, |
| | ovarian, pancreatic, prostate, renal, skin, uterine |
| PAX2 | Breast |
| FZD | Liver |
| ARG2 | Breast, non small cell lung |
| eIF5A1 | Cancer |
| Atg1, Atg2, Atg3, Atg4, Atg5, | Breast, liver, ovarian, gastric, bladder, |
| Beclin1, Atg7, MAP1 LC3B, | colon, prostate, lung, nasopharyngeal carcinoma, |
| Atg9/APG9L1/2, Atg10, Atg12, Atg16, | neuroblastoma, glioma, solid tumor, hematologic |
| mTOR, PIK3C3, VPS34 | malignancy, leukemia, lymphoma |
| SEPT10, LMNB2, HRH1, | Colon, osteosarcoma, liver, melanoma, |
| HOXA10, ERCC3, MIS12, MPHOSPHI1, | head and neck squamous cell carcinoma |
| CDC7, SMARCB1, MAD2L1, DTL, | |
| RACGAP1, MCM10, PIM1, DLG5, BCL2, | |
| CUL5, PRPF38A | |
| Cineurin | Leukemia, lymphoma, melanoma, lung, |
| | bowel, colon, rectal, colorectal, brain, liver, |
| | pancreatic, breast, testicular, retinoblastoma |
| alpha-enolase | Cancer |
| BRAF | Malignant melanoma |
| Androgen receptor | Bladder |
| HOXB13 | Prostate |
| Wnt2 | Breast, ovarian, colorectal, gastric, lung, |
| | kidney, bladder, prostate, uterine, thyroid, |
| | pancreatic, cervical, esophageal, mesothelioma, |
| | head and neck, hepatocellular, melanoma, brain |
| | vulval, testicular, sarcoma, intestine, skin, |
| | leukemia, lymphoma |
| NuMA | Cervical, epidermoid, oral, glioma, |
| | leukemia, brain, esophageal, stomach, bladder, |
| | pancreatic, cervical, head and neck, ovarian, |
| | melanoma, lymphoma |
| Ang-1, Ang-2, Tie2 | Cancer |
| MAGE-B (B1, B2, B3, B4), | Melanoma, lymphoma, T cell leukemia, |
| MAGE-C, MAG-A(A1, A3, A5, A6, A8, | non small cell lung, hepatic carcinoma, gastric, |
| A9, A10, A11, A12), Necdin, MAGE-D, | esophagus, colorectal, gastric, endocrine, ovarian, |
| MAGE-E (E1), MAGE-F, MAGE-G, | pancreatic, ovarian, cervical, salivary, head and |
| MAGE-H | neck squamous cell, spermatocytic seminoma, |
| | sporadic medulalry thyroid carcinoma, bladder, |
| | osteosarcoma, non-proliferating testes cells, |
| | neuroblastoma, glioma, cancers related to |
| | malignant mast cells |
| Galactin-1 | Glioma, pancreatic, non small cell lung, |
| | non-Hodgkin's lymphoma |
| Tpt1 | Cancer |
| c-FLIP | Cancer |
| EBAG9 | Prostate, bladder |
| Nrf2 | Lung |
| E6TMF/ARA160 | Cancer |
| Jun, Erg-2 | Cancer |
| CSN5 | Hepatocellular Carcinoma |
| COP1-1 | Hepatocellular Carcinoma |
| PLK1 | Cancer |
| LMP2, LMP7, MECL1 | Metastatic melanoma |
| M2 subunit ribonucleotide | Solid tumor |
| reductase | |
| AHR | Neuroblastoma |
| B4GALNT3 | Neuroblastoma |
| PKN3 | Colorectal cancer metastasizing to the liver |
| KSP | Liver cancer |
| b-catenin | Familial adenomatous polyposis |

### Inflammation and Autoimmune Disease

In another aspect, the disclosure features, a method of treating or preventing a disease or disorder in a subject wherein the disease or disorder is inflammation or an autoimmune disease. The method comprises administering an effective amount of a nucleic acid-hydrophobic polymer conjugate, particle, or composition described herein to thereby treat the disease or disorder. In an embodiment the nucleic acid-hydrophobic polymer conjugates, particles and compositions can be used as part of a first line, second line, or adjunct therapy, and can also be used alone or in combination with one or more additional treatment regimes.

In an embodiment the nucleic acid-hydrophobic polymer conjugates, particles, compositions and methods described herein can be used to treat or prevent a disease or disorder associated with inflammation. In embodiments a nucleic acid-hydrophobic polymer conjugate, particle or composition described herein may be administered prior to the onset of, at, or after the initiation of inflammation. In embodiments, used prophylactically, the nucleic acid-hydrophobic polymer conjugate, particle or composition is provided in advance of any inflammatory response or symptom. In embodiments administration of the nucleic acid-hydrophobic polymer conjugate, particle or composition can prevent or attenuate inflammatory responses or symptoms. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

Other examples of inflammatory conditions include: inflammation associated with acne; anemia (e.g., aplastic anemia, haemolytic autoimmune anaemia); asthma; arteritis (e.g., polyarteritis, temporal arteritis, periarteritis nodosa, Takayasu's arteritis); arthritis (e.g., crystalline arthritis, osteoarthritis, psoriatic arthritis, gouty arthritis, reactive arthritis, rheumatoid arthritis and Reiter's arthritis); ankylosing spondylitis; amylosis; amyotrophic lateral sclerosis; allergies or allergic reactions; Alzheimer's disease; atherosclerosis; bronchitis; bursitis; chronic prostatitis; conjunctivitis; Chagas disease; chronic obstructive pulmonary disease; cermatomyositis; diverticulitis; diabetes (e.g., type I diabetes mellitus, type 2 diabetes mellitus); dermatitis; eosinophilic gastrointestinal disorders (e.g., eosinophilic esophagitis, eosinophilic gastritis, eosinophilic gastroenteritis, eosinophilic colitis); eczema; endometriosis; gastrointestinal bleeding; gastritis; gastroesophageal reflux disease (GORD, or its synonym GERD); Guillain-Barre syndrome; infection; ischaemic heart disease; Kawasaki disease; glomerulonephritis; gingivitis; hypersensitivity; headaches (e.g., migraine headaches, tension headaches); ileus (e.g., postoperative ileus and ileus during sepsis); idiopathic thrombocytopenic purpura; interstitial cystitis; inflammatory bowel disease (IBD) (e.g., Crohn's disease, ulcerative colitis, collagenous colitis, lymphocytic colitis, ischaemic colitis, diversion colitis, Behcet's syndrome, indeterminate colitis); inflammatory bowel syndrome (IBS); lupus; multiple sclerosis; morphea; myeasthenia gravis; myocardial ischemia; nephrotic syndrome; pemphigus vulgaris; pernicious anemia; peptic ulcers; psoriasis; polymyositis; primary biliary cirrhosis; Parkinson's disease; pelvic inflammatory disease; reperfusion injury; regional enteritis; rheumatic fever; systemic lupus erythematosus; schleroderma; scierodoma; sarcoidosis; spondyloarthopathies; Sjogren's syndrome; thyroiditis; transplantation rejection; tendonitis; trauma or injury (e.g., frostbite, chemical irritants, toxins, scarring, burns, physical injury); vasculitis; vitiligo; and Wegener's granulomatosis.

In another embodiment, a nucleic acid-hydrophobic polymer conjugate, particle, composition or method described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The nucleic acid-hydrophobic polymer conjugate, particle or composition may be used to treat chronic hepatitis infection, including hepatitis B and hepatitis C.

In embodiments a nucleic acid-hydrophobic polymer conjugate, particle, composition or method described herein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

Other examples of autoimmune disorders include: but are not limited to: acute disseminated encephalomyelitis (ADEM); Addison's disease; antiphospholipid antibody syndrome (APS); aplastic anemia; autoimmune hepatitis; cancer; coeliac disease; Crohn's disease; Diabetes mellitus (type 1); Goodpasture's syndrome; Graves' disease; Guillain-Barre syndrome (GBS); Hashimoto's disease; lupus erythematosus; multiple sclerosis; myasthenia gravis; opsoclonus myoclonus syndrome (OMS); optic neuritis; Ord's thyroiditis; oemphigus; polyarthritis; primary biliary cirrhosis; psoriasis; rheumatoid arthritis; Reiter's syndrome; Takayasu's arteritis; temporal arteritis (also known as “giant cell arteritis”); warm autoimmune hemolytic anemia; Wegener's granulomatosis; alopecia universalis; Chagas disease; chronic fatigue syndrome; dysautonomia; endometriosis; hidradenitis suppurativa; interstitial cystitis; neuromyotonia; sarcoidosis; scleroderma; ulcerative colitis; vitiligo; and vulvodynia.

In an embodiment, a nucleic acid-hydrophobic polymer conjugate, particle or composition, e.g., containing an siRNA that targets a gene listed in Table 4, is administered, e.g, to treat or prevent, an associated disease listed in Table 4.

TABLE 4
The nucleic acid, e.g. nucleic acid agent, e.g., an siRNA, can target a gene
listed in the table, for example, to treat or prevent the associated disease.
Inflammatory/Autoimmune Diseases

| Gene | Diseases |
|---|---|
| ICAM-1 | Inflammatory skin diseases (allergic |
| | contact dermatitis, fixed drug eruption, lichen |
| | planus, psoriasis), asthma, allergic rhinitis, |
| | allergic conjunctivitis, immune based nephritis, |
| | contact dermal hypersensitivity, type 1 diabetes, |
| | inflammatory lung diseases, inflammatory bowel |
| | disease, inflammatory skin disorders, allograft |
| | rejection, immune cell interactions, mixed t cell |
| | reaction, meningitis, multiple sclerosis, |
| | rheumatoid arthritis, septic arthritis, uveitis, age |
| | related macular degeneration |
| IL-18 | Chronic Obstructive Pulmonary Disease |
| | (COPD) |
| IFNgamma | COPD |
| PKR | COPD |
| VEGF | Preventing post operative |
| | neovascularization and post operative |
| | inflammation in ophthalmic |
| IL2R | Lupus, nephritis, inflammatory bowel |
| | disease, inflammation associated with |
| | transplanted |
| NPRA | Respiratory allergy, viral infection |
| FIZZ1 | Airway inflammation |
| Akt & p85alpha | Inflammatory bowel disease, chronic |
| | inflammatory state associated with organ |
| | transplants, pancreatitis, arthritis, enterocolitis, |
| | autoimmune disease, chronic inflammatory state |
| | associated with infection, toxin, allergy |
| TREM-1 | Asthma, rheumatoid arthritis |
| BIM, PUMA, BAX, BAK | Sepsis |
| STAT6 | Asthma, non-atopic asthma, rhinitis |
| BLT2 | Asthma |
| FCepsilonR alpha chain, | Allergic rhinitis, asthma |
| FCepsilonRbeta chain, c-Kit, LYN, SYK, | |
| ICOS, OX40L, CD40, CD80, CD86, RELA, | |
| RELB, 4-1BB ligand, TLR1, TLR2, TLR3, | |
| TLR5, TLR6, TLR7, TLR8, TLR9, CD83, | |
| SLAM, common gamma chain, COX2 | |
| IL-1, IL-2, IL-3, IL4, IL-5, IL-6, IL- | Allergic rhinitis, asthma, COPD |
| 7, IL-8, IL-9, IL-10, IL-11, IL-12, IL-13, | |
| IL-14, IL-15, IL-16, IL-17, IL-18, IL-19, | |
| IL-20, IL-21, IL-22, IL-23, IL-24, IL-25, | |
| IL-26, IL-27, IL-1R, IL-2R, IL-3R, IL4R, | |
| IL-5R, IL-6R, IL-7R, IL-8R, IL-9R, IL-10R, | |
| IL-11R, IL-12R, IL-13R, IL-14R, IL-15R, | |
| IL-16R, IL-17R, IL-18R, IL-19R, IL-20R, | |
| IL-21R, IL-22R, IL-23R, IL-24R, IL-25R, | |
| IL-26R, IL-27R | |
| Calpain 1 & Calpain 2 | Asthma, asthma exacerbation, chronic |
| | obstructive pulmonary disease, opportunistic |
| | pathogenic infection of cystic fibrosis, respiratory |
| | infection, pneumonia, ventilator associated |
| | pneumonia, obstructive airway disease, bronchial |
| | condition, pulmonary inflammation, eosinophil |
| | related disorder |
| IL-1, TNFalpha, Fas, FasL | Hepatitis, cirrhosis, transplant rejection |
| IL-1, IL-2, IL-4, IL-7, IL-12, IFNs, | Rheumatoid arthritis, chron's disease, |
| GMCSF, TNFalpha | multiple sclerosis, psoriasis |
| ICAM1, VCAM1, IFN gamma, IL- | Suppressing rejection of transplanted |
| 1, IL-6, IL-8, TNFalpha, CD8-, CD86, | organ by a recipient of the organ |
| MHC-II, MHC-I, CD28, CTLA4, PV-B19 | |
| TGFB1, COX2 | Wound healing |
| Cyclin D1 | Inflammatory bowel disease, ulcerative |
| | colitis, crohn's disease, celiac disease, |
| | autoimmune hepatitis, chronic rheumatoid |
| | arthritis, psoratic arthritis, insulin dependent |
| | diabetes mellitus, multiple sclerosis, enterogenic |
| | spondyloarthropathies, autoimmune myocarditis, |
| | psoriasis, scleroderma, myasthenia gravis, |
| | multiple myostisis/dermatomyostisis, hashimoto's |
| | disease, autoimmune hypocytosis, pure red cell |
| | apalsia, aplastic anemia, sjogren's syndrome, |
| | vascultis syndrome, systemic lupus |
| | erythematosus, glomerulonephritis, pulmonary |
| | inflammation, septic shock, transplant rejection |

### Cardiovascular Disease

In another aspect, the disclosure features, a method of treating or preventing a disease or disorder in a subject wherein in the disorder is a cardiovascular disease. The method comprises administering an effective amount of a nucleic acid-hydrophobic polymer conjugate, particle, or composition described herein to thereby treat the disease or disorder. In an embodiment the nucleic acid-hydrophobic polymer conjugates, particles and compositions can be used as part of a first line, second line, or adjunct therapy, and can also be used alone or in combination with one or more additional treatment regimes.

In embodiments the disclosed methods may be useful in the prevention and treatment of cardiovascular disease. Cardiovascular diseases that can be treated or prevented using nucleic acid-hydrophobic polymer conjugates, particles, compositions and methods described herein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using nucleic acid-hydrophobic polymer conjugates, particles, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. In embodiments other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The nucleic acid-hydrophobic polymer conjugates, particles, compositions and methods described herein may also be used for increasing HDL levels in plasma of an individual.

Yet other disorders that may be treated with nucleic acid-hydrophobic polymer conjugates, particles, compositions and methods described herein include restenosis, e.g., following coronary intervention, and disorders relating to an abnormal level of high density and low density cholesterol.

In embodiments the nucleic acid-hydrophobic polymer conjugate, particle or composition can be administered to a subject undergoing or who has undergone angioplasty. In one embodiment, the nucleic acid-hydrophobic polymer conjugate, particle or composition is administered to a subject undergoing or who has undergone angioplasty with a stent placement. In some embodiments, the nucleic acid-hydrophobic polymer conjugate, particle or composition can be used as a coating for a stent.

In embodiments the nucleic acid-hydrophobic polymer conjugates, particles or compositions can be used during the implantation of a stent, e.g., as a separate intravenous administration, as a coating for a stent.

Examples of cardiovascular diseases include, but are not limited to: angina; arrhythmias (atrial or ventricular or both), or long-standing heart failure; arteriosclerosis; atheroma; atherosclerosis; cardiac hypertrophy including both atrial and ventricular hypertrophy; cardiac or vascular aneurysm; cardiac myocyte dysfunction; carotid obstructive disease; congestive heart failure; endothelial damage after PTCA (percutaneous transluminal coronary angioplasty); hypertension including essential hypertension, pulmonary hypertension and secondary hypertension (renovascular hypertension, chronic glomerulonephritis); myocardial infarction; myocardial ischemia; peripheral obstructive arteriopathy of a limb, an organ, or a tissue; peripheral artery occlusive disease (PAOD); reperfusion injury following ischemia of the brain, heart or other organ or tissue; restenosis; stroke; thrombosis; transient ischemic attack (TIA); vascular occlusion; vasculitis; and vasoconstriction.

In some embodiments, the cardiovascular disease can be an inflammatory disease of the heart such as cardiomyopathy, ischemic heart disease, hypercholesterolemia, and atherosclerosis.

In an embodiment, a nucleic acid-hydrophobic polymer conjugate, particle or composition, e.g., containing an siRNA that targets a gene listed in Table 5, is administered, e.g, to treat or prevent, an associated disease listed in Table 5.

TABLE 5
The nucleic acid, e.g. nucleic acid agent, e.g., an siRNA, can target a gene
listed in the table, for example, to treat or prevent the associated disease.
Cardiovascular Diseases

| Gene | Diseases |
|---|---|
| ICAM-1 | Atherosclerosis, myocarditis, pulmonary |
| | fibrosis |
| S1P2 & Caspase 11 | Heart disease, stroke, peripheral vascular |
| | disease, vasculitis |
| ApoB | Hypercholesterolemia, atherosclerosis, |
| | angina pectoris, high blood pressure, diabetes, |
| | hypothyroidism |
| KLF5 | Arteriosclerosis, restenosis occurring after |
| | coronary intervention, cardiac hypertrophy |
| CETP | Cardiovascular disorders |
| PLOD2 | Fibrotic tissue formation occurring in |
| | myocardial infarct related fibrosis, cardiac fibrosis, |
| | valvular stenosis, intimal hyperplasia, diabetic |
| | ulcers, peridural fibrosis, perineural fibrosis |
| Ku | Cardiac hypertrophy, heart failure |
| Agtr11, Apelin, | Cardiovascular disease, atherosclerosis, |
| Stabilin 1, | atherosclerotic plaque formation, plaque |
| Stabilin 2, | destabilization, vulnerable plaque formation and |
| TNFaip811, | rupture |
| TNFaip8, | |
| FGD5 | |
| ROCK1 | Cardiac failure |
| PCSK9, | Heart disease |
| apolipoprotein B | Cardiovascular disease, angina pectoris, |
| sNRF | arrhythmia, cardiac fibrosis, congenital |
| | cardiovascular disease, coronary artery disease, |
| | dilated cardiomyopathy, myocardial infarction, |
| | heart failure, hypertrophic cardiomyopathy, |
| | systemic hypertension from any cause, edematous |
| | disorders caused by liver or renal disease, mitral |
| | regurgitation, myocardial tumors, myocarditis, |
| | rheumatic fever, Kawasaki disease, Takaysu |
| | arteritis, cor pulmonale, primary pulmonary |
| | hypertension, amyloidosis, hemachromatosis, toxic |
| | effects on the heart due to poisoning, Chaga's |
| | disease, heart transplantation, cardiac rejection |
| | after heart transplant, cardiomyopathy of |
| | chachexia, arrhythmogenic right ventricular |
| | dysplasia, cardiomyopathy of pregnancy, Marfan |
| | Syndrome, Turner syndrome, Loeys-Dietz |
| | Syndrome, familial bicuspid aortic valve |

### Metabolic Disorders

The disclosure also features the use of nucleic acid-polymer conjugates described herein for the treatment or prevention of a metabolic disorder in a subject, e.g., a human subject. The term “metabolic disorder” includes a disorder, disease or condition which is caused or characterized by an abnormal metabolism (i.e., the chemical changes in living cells by which energy is provided for vital processes and activities) in a subject. Examples of disorders include obesity, diabetes, a co-morbidity of obesity, and an obesity related disorder. The subject to whom the polymer-agent, particle or composition is administered may be overweight or obese. Alternatively, or in addition, the subject may be diabetic, for example having insulin resistance or glucose intolerance, or both. The subject may have diabetes mellitus, for example, the subject may have Type II diabetes. The subject may be overweight or obese and have diabetes mellitus, for example, Type II diabetes.

In addition, or alternatively, the subject may have, or may be at risk of having, a disorder in which obesity or being overweight is a risk factor. As used herein, “obesity” refers to a body mass index (BMI) of 30 kg/m² or more (National Institute of Health, Clinical Guidelines on the Identification, Evaluation, and Treatment of Overweight and Obesity in Adults (1998)). However, the disclosure is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, all of which are typically referred to as overweight (National Institute of Health, Clinical Guidelines on the Identification, Evaluation, and Treatment of Overweight and Obesity in Adults (1998)). Such disorders include, but are not limited to, cardiovascular disease, for example hypertension, atherosclerosis, congestive heart failure, and dyslipidemia; stroke; gallbladder disease; osteoarthritis; sleep apnea; reproductive disorders for example, polycystic ovarian syndrome; cancers, for example breast, prostate, colon, endometrial, kidney, and esophagus cancer; varicose veins; acanthosis nigricans; eczema; exercise intolerance; insulin resistance; hypertension; hypercholesterolemia; cholithiasis; osteoarthritis; orthopedic injury; insulin resistance, for example, type 2 diabetes and syndrome X; metabolic syndrome; and thromboembolic disease (see Kopelman (2000), Nature 404:635-43; Rissanen et al., British Med. J. 301, 835, 1990).

Other disorders associated with obesity include depression, anxiety, panic attacks, migraine headaches, PMS, chronic pain states, fibromyalgia, insomnia, impulsivity, obsessive-compulsive disorder, irritable bowel syndrome (IBS), and myoclonus. Furthermore, obesity is a recognized risk factor for increased incidence of complications of general anesthesia. (See e.g., Kopelman, Nature 404:635-43, 2000). In general, obesity reduces life span and carries a serious risk of co-morbidities such as those listed above.

Other diseases or disorders associated with obesity are birth defects, maternal obesity being associated with increased incidence of neural tube defects, carpal tunnel syndrome (CTS); chronic venous insufficiency (CVI); daytime sleepiness; deep vein thrombosis (DVT); end stage renal disease (ESRD); gout; heat disorders; impaired immune response; impaired respiratory function; infertility; liver disease; lower back pain; obstetric and gynecologic complications; pancreatititis; as well as abdominal hernias; acanthosis nigricans; endocrine abnormalities; chronic hypoxia and hypercapnia; dermatological effects; elephantitis; gastroesophageal reflux; heel spurs; lower extremity edema; mammegaly which causes considerable problems such as bra strap pain, skin damage, cervical pain, chronic odors and infections in the skin folds under the breasts, etc.; large anterior abdominal wall masses, for example abdominal panniculitis with frequent panniculitis, impeding walking, causing frequent infections, odors, clothing difficulties, lower back pain; musculoskeletal disease; pseudo tumor cerebri (or benign intracranial hypertension), and sliding hiatil hernia.

Conditions or disorders associated with increased caloric intake include, but are not limited to, insulin resistance, glucose intolerance, obesity, diabetes, including type 2 diabetes, eating disorders, insulin-resistance syndromes, metabolic syndrome X, and Alzheimer's disease.

### Central Nervous System Disorders

The disclosure also features the use of nucleic acid-polymer conjugates, particles, and compositions as described herein for the treatment of central nervous system disorders in a subject, e.g., a human subject. Examples of central nervous system disorders include, but are not limited to: a myelopathy; an encephalopathy; central nervous system (CNS) infection; encephalitis (e.g., viral encephalitis, bacterial encephalitis, parasitic encephalitis); meningitis (e.g., spinal meningitis, bacterial meningitis, viral meningitis, fungal meningitis); neurodegenerative diseases (e.g., Huntington's disease; Alzheimer's disease; Parkinson's disease; multiple sclerosis; amyotrophic lateral sclerosis; traumatic brain injury); mental health disorder (e.g., schizophrenia, depression, dementia); pain and addiction disorders; brain tumors (e.g., intra-axial tumors, extra-axial tumors); adult brain tumors (e.g., glioma, glioblastoma); pediatric brain tumors (e.g., medulloblastoma); cognitive impairment; genetic disorders (e.g., Huntington's disease, neurofibromatosis type 1, neurofibromatosis type 2, Tay-Sachs disease, tuberous sclerosis); headache (e.g., tension headache; migraine headache, cluster headache, meningitis headache, cerebral aneurysm and subarachnoid hemorrhage headache, brain tumor headache); stroke (e.g., cerebral ischemia or cerebral infarction, transient ischemic attack, hemorrhagic (e.g., aneurysmal subarachnoid hemorrhage, hypertensive hemorrhage, other sudden hemorrhage)); epilepsy; spinal disease (e.g., degenerative spinal disease (e.g., herniated disc disease, spinal stenosis, and spinal instability), traumatic spine disease; spinal cord trauma; spinal tumors; hydrocephalus (e.g., communicating or non-obstructive hydrocephalus, non-communicating or obstructive hydrocephalus, adult hydrocephalus, pediatric hydrocephalus, normal pressure hydrocephalus, aqueductal stenosis, tumor associated hydrocephalus, pseudotumor cerebri); CNS vasculitis (e.g., primary angiitis of the central nervous system, benign angiopathy of the central nervous system; Arnold Chiari malformation; neuroAIDS; retinal disorders (e.g., age-related macular degeneration, wet age-related macular degeneration, myopic macular degeneration, retinitis pigmentosa, proliferative retinopathies); inner ear disorders; tropical spastic paraparesis; arachnoid cysts; locked-in syndrome; Tourette's syndrome; adhesive arachnoiditis; altered consciousness; autonomic neuropathy; benign essential tremor; brain anomalies; cauda equine syndrome with neurogenic bladder; cerebral edema; cerebral spasticity; cerebral vascular disorder; and Guillain-Barre syndrome.

### Neurological Deficits

The nucleic acid-polymer conjugates, particles, and compositions can be used to treat neurological deficits due to neurodegeneration in the brain of a subject, e.g., a human subject. The method can include administering a polymer-agent, particle or composition described herein to the subject. As used herein, the phrase “neurological deficits” includes an impairment or absence of a normal neurological function or presence of an abnormal neurological function. Neurodegeneration of the brain can be the result of disease, injury, and/or aging. As used herein, neurodegeneration includes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, head trauma, stroke, ALS, multiple sclerosis, Huntington's disease, Parkinson's disease, and Alzheimer's disease.

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

Unless otherwise defined, all technical and scientific terms used herein have the same meaning as commonly understood by one of ordinary skill in the art to which this disclosure belongs. All publications, patent applications, patents, and other references mentioned herein are incorporated by reference in their entirety. In case of conflict, the specification, including definitions, will control. In addition, the materials, methods, and examples are illustrative only and not intended to be limiting.

### EXAMPLES

The disclosure is further described in the following examples, which do not limit the scope of the disclosure described in the claims.

### Example 1

### Purification and characterization of 5050 PLGA

Step A: A 3-L round-bottom flask equipped with a mechanical stirrer was charged with 5050PLGA (300 g, Mw: 7.8 kDa; Mn: 2.7 kDa) and acetone (900 mL). The mixture was stirred for 1 h at ambient temperature to form a clear yellowish solution.
Step B: A 22-L jacket reactor with a bottom-outlet valve equipped with a mechanical stirrer was charged with MTBE (9.0 L, 30 vol. to the mass of 5050 PLGA). Celite® (795 g) was added to the solution with overhead stirring at ˜200 rpm to produce a suspension. To this suspension was slowly added the solution from Step A over 1 h. The mixture was agitated for an additional one hour after addition of the polymer solution and filtered through a polypropylene filter. The filter cake was washed with MTBE (3×300 mL), conditioned for 0.5 h, air-dried at ambient temperature (typically 12 h) until residual MTBE was ≦5 wt % (as determined by ¹H NMR analysis).
Step C: A 12-L jacket reactor with a bottom-outlet valve equipped with a mechanical stirrer was charged with acetone (2.1 L, 7 vol. to the mass of 5050 PLGA). The polymer/Celite® complex from Step B was charged into the reactor with overhead stirring at ˜200 rpm to produce a suspension. The suspension was stirred at ambient temperature for an additional 1 h and filtered through a polypropylene filter. The filter cake was washed with acetone (3×300 mL) and the combined filtrates were clarified through a 0.45 mM in-line filter to produce a clear solution. This solution was concentrated to ˜1000 mL.
Step D: A 22-L jacket reactor with a bottom-outlet valve equipped with a mechanical stirrer was charged with water (9.0 L, 30 vol.) and was cooled down to 0-5° C. using a chiller. The solution from Step C was slowly added over 2 h with overhead stirring at ˜200 rpm. The mixture was stirred for an additional one hour after addition of the solution and filtered through a polypropylene filter. The filter cake was conditioned for 1 h, air-dried for 1 day at ambient temperature, and then vacuum-dried for 3 days to produce the purified 5050 PLGA as a white powder [258 g, 86% yield]. The ¹H NMR analysis was consistent with that of the desired product and Karl Fisher analysis showed 0.52 wt % of water. The product was analyzed by HPLC (AUC, 230 nm) and GPC (AUC, 230 nm). The process produced a narrower polymer polydispersity, i.e. Mw: 8.8 kDa and Mn: 5.8 kDa.

### Example 2

### Synthesis, purification and characterization of O-acetyl-5050-PLGA

A 2000-mL, round-bottom flask equipped with an overhead stirrer was charged with purified 5050 PLGA [220 g, Mn of 5700] and DCM (660 mL). The mixture was stirred for 10 min to form a clear solution. Ac₂O (11.0 mL, 116 mmol) and pyridine (9.4 mL, 116 mmol) were added to the solution, resulting in a minor exotherm of ˜0.5° C. The reaction was stirred at ambient temperature for 3 h and concentrated to ˜600 mL. The solution was added to a suspension of Celite® (660 g) in MTBE (6.6 L, 30 vol.) over 1 h with overhead stirring at ˜200 rpm. The suspension was filtered through a polypropylene filter and the filter cake was air-dried at ambient temperature for 1 day. It was suspended in acetone (1.6 L, ˜8 vol) with overhead stirring for 1 h. The slurry was filtered though a fritted funnel (coarse) and the filter cake was washed with acetone (3×300 mL). The combined filtrates were clarified though a Celite® pad to afford a clear solution. It was concentrated to ˜700 mL and added to cold water (7.0 L, 0-5° C.) with overhead stirring at 200 rpm over 2 h. The suspension was filtered though a polypropylene filter. The filter cake was washed with water (3×500 mL), and conditioned for 1 h to afford 543 g of wet cake. It was transferred to two glass trays and air-dried at ambient temperature overnight to afford 338 g of wet product, which was then vacuum-dried at 25° C. for 2 days to constant weight to afford 201 g of product as a white powder [yield: 91%]. The ¹H NMR analysis was consistent with that of the desired product. The product was analyzed by HPLC (AUC, 230 nm) and GPC (Mw: 9.0 kDa and Mn: 6.3 kDa).

### Example 3

### Synthesis, purification, and characterization of 2-(2-(Pyridin-2-yl)disulfanyl)ethylamine

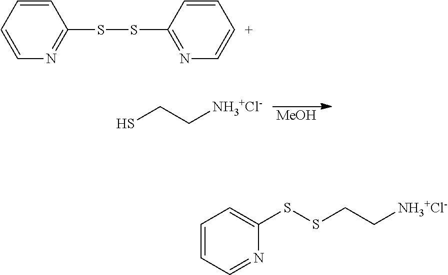

In a 25 mL round bottom flask, 2,2′-dithiodipyridine (2.0 g, 9.1 mmol) was dissolved in methanol (8 mL) with acetic acid (0.3 mL). Cysteamine hydrochloride (520 mg, 4.5 mmol) was dissolved in methanol (5 mL) and added dropwise into the mixture over ½ h. The mixture was stirred overnight. It was then concentrated under vacuum to yield yellow oil. The oil was dissolved back in methanol (5 mL) and then precipitated into diethyl ether (100 mL). The precipitate was filtered off and dried. It was then redissolved in methanol (5 mL) and reprecipitated in diethyl ether (100 mL). This procedure was repeated for two more times. The pale yellow solid was filtered off and dried to yield the final product (0.74 g, 74% yield) which was used without further purification. The ¹H NMR analysis was consistent with that of the desired product.

### Example 4

### Synthesis, purification, and characterization of 2-(2-(pyridin-2-yl)disulfanyl)ethylamino-5050-PLGA-O-acetyl

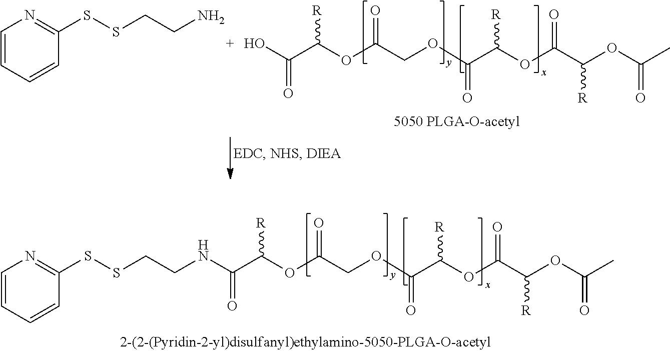

In a 50 mL round bottom flask, 5050 PLGA_{6.3k}-O-acetyl (2.0 g, 0.32 mmol), NHS (66 mg, 0.57 mmol) and EDC (122 mg, 0.63 mmol) was dissolved in DMF (12 mL). To the reaction mixture, 2-(2-(pyridin-2-yl)disulfanyl)ethylamine (127 mg, 0.57 mmol) and diisopropylethylamine (82 mg, 0.63 mmol) in DMF (6 mL) were added. The reaction mixture was then stirred at room temperature for 4 h. Water (40 mL) was added to the reaction mixture to give a gummy solid. The gummy solid was dissolved in DCM (15 mL) and washed twice with 0.1% aqueous HCl solution (50 mL×2) followed by brine (100 mL). The organic layer was dried over sodium sulphate and further purified by precipitation into cold ether (100 mL). Solvent was removed and the material was dried under vacuum to yield white solid (1.4 g, 68% yield). The ¹H NMR analysis was consistent with that of the desired product.

### Example 5

### Synthesis, purification, and characterization of oligonucleotide-C6-SS-5050 PLGA-O-acetyl

C6-thiol modified oligonucleotides (siRNA, 0.2 mg, 14.7 nmol) were conjugated to 2-(2-(pyridin-2-yl)disulfanyl)ethylamino-5050-PLGA-O-acetyl (10 mg, 1.58 μmol) as prepared in Example 4 in a solvent mixture of 95:5 DMSO:TE buffer (1 mL). The reaction mixture was stirred at 65° C. for 2 hours. The oligonucleotide-5050-PLGA-O-acetyl conjugate was analyzed by reverse phase HPLC and gel electrophoresis.

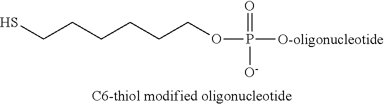

### Example 6

### Synthesis, purification, and characterization of oligonucleotide-C6-SS-5050 PLGA-O-acetyl

C6-thiol modified oligonucleotides against EGFP (enhanced green fluorescent protein) having a Mw of 13.2 kDa (siRNA, 20 mg, 1.51 μmol) with sense strands having nucleotide sequences substantially identical to a portion of the EGFP sequence, being 19 base pairs in length with a UU overhang, and having complementary antisense strands, were conjugated to 2-(2-(Pyridin-2-yl)disulfanyl)ethylamino-5050-PLGA-O-acetyl (85 mg, 11 μmol) as prepared in Example 4 in a solvent mixture of 95:5 DMSO:TE buffer (10 mL). The reaction mixture was stirred at 65° C. for 3 hours. The oligonucleotide-5050-PLGA-O-acetyl conjugate was analyzed by reverse phase HPLC and gel electrophoresis.

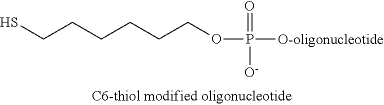

### Example 7

### Synthesis, purification, and characterization of oligonucleotide-C6-SS-5050 PLGA-O-acetyl

C6-thiol modified oligonucleotides against luciferase (siRNA, 20 mg, 1.51 μmol, Mw of 13.6 kDa) with sense strands having nucleotide sequences substantially identical to a portion of the luciferase sequence, being 19 base pairs in length with a UU overhang, and having complementary antisense strands, were conjugated to 2-(2-(Pyridin-2-yl)disulfanyl)ethylamino-5050-PLGA-O-acetyl (85 mg, 11 μmol) as prepared in Example 4 in a solvent mixture of 95:5 DMSO:TE buffer (10 mL). The reaction mixture was stirred at 65° C. for 3 hours. The oligonucleotide-5050-PLGA-O-acetyl conjugate was analyzed by reverse phase HPLC and gel electrophoresis.

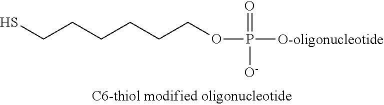

### Example 8

### Synthesis, purification, and characterization of azide terminated-PEG linker-5050 PLGA-O-acetyl

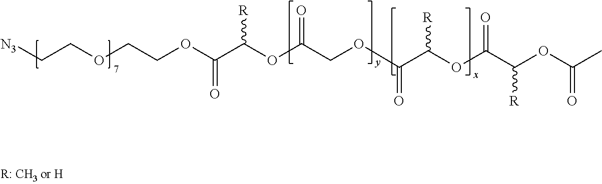

In a 50 mL round bottom flask, 5050 PLGA-O-acetyl (2.0 g, 0.13 mmol) will be dissolved in DCM (10 mL). To the reaction mixture, azide-PEG₈-OH (40 mg, 0.13 mmol), NHS (29 mg, 0.25 mmol) and EDC (39 mg, 0.25 mmol) will be added. It was then stirred at RT for 4 h. Cold Et₂O (100 mL) will then be added to the reaction mixture to precipitate out the polymer. The precipitated polymer will be dried under vacuum to yield a white foam. The ¹H NMR analysis will be carried out to determine the identity of the desired compound.

### Example 9

### Synthesis, purification, and characterization of oligonucleotide-C6-triazole-PEG-5050 PLGA-O-acetyl

10 μL precomplexed Cu(I) will be added (10 mM; 1 mg CuBr (99.99%) dissolved in 700 μL of 10 mM TBTA tris(benzyltriazolylmethyl)amine ligand in tert-BuOH:DMSO 1:3) to a reaction mixture of C6-alkyne-modified oligonucleotides (siRNA or DNA) (1 to 4 pmol siRNA or DNA, 10 mM Tris) and azide terminated-PEG-5050 PLGA-O-acetyl solution (10 μL of 5 mM, diluted with 10 mM Tris with 5% tBuOH from a stock of 0.1 N in DMSO) (Example 8). The sample will be stirred at room temperature for 2 hours. The reaction mixture will be analyzed by anionic-exchange and reversed phase HPLC.

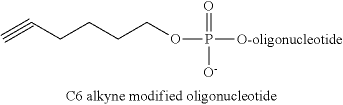

### Example 10

### Synthesis, purification, and characterization of oligonucleotide-PEG-triazole-PEG-5050 PLGA-O-acetyl

10 μL precomplexed Cu(I) will be added (10 mM; 1 mg CuBr (99.99%) dissolved in 700 μL of 10 mM TBTA tris(benzyltriazolylmethyl)amine ligand in tert-BuOH:DMSO 1:3) to a reaction mixture of alkyne-PEG-modified oligonucleotides (siRNA or DNA) (1 to 4 pmol siRNA or DNA, 10 mM Tris) and azide terminated-PEG-5050 PLGA-O-acetyl solution (10 μL of 5 mM, diluted with 10 mM Tris with 5% tBuOH from a stock of 0.1 N in DMSO) (Example 8). The sample will be stirred at room temperature for 2 hours. The reaction mixture will be analyzed by anionic-exchange and reversed phase HPLC.

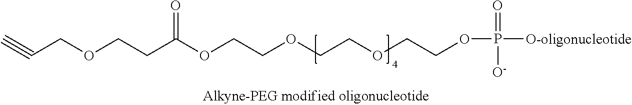

### Example 11

### Separation of an oligonucleotide-C6-SS-5050 PLGA Conjugate

### Materials

siRNA of several 20-21 base pair sequences was received from Thermo Fisher with a reduced hexyl thiol end group on the sense strand. The siRNA was conjugated to the activated PLGA polymer, PLGA-SS-pyridine from Albany Molecular Research, Inc. (Albany N.Y.), on a 1:10 of siRNA to activated PLGA polymer ratio in 95% DMSO (ACS grade) 5% TE buffer. Mobile Phase components DMF, ACN, and water were HPLC grade from Baker and anhydrous LiBr was ACS grade.

### Run Parameters

5 mL scale
Column: Inertsil DIOL 300A<USP L20 classification
Mobile Phase: acetonitrile, 20 mM LiBr in N,N-dimethylformamide, and water
Column length: 150 mm

### Column ID: 4.6 mm

No. of columns: 1
Sample concentration: 40 mg/mL RNA

### Temperature: 25° C.

| | |
|---|---|
| Flow rate (ml/min) | 0.750 |
| Run Time (min) | 17 |
| Pressure (max.) (bar) | 60 |
| Pressure (min.) (bar) | 30 |

### Methods

Analyses of the nucleic acid-hydrophobic polymer conjugates were performed on an Agilent 1200 series automated HPLC equipped with a quaternary pump, diode array detector and autosampler with thermostatic control. Separations were made on a GL sciences Inertsil® DIOL column 4.6×150 mm part #1290L150W046, held at 25° C. The flow rate was 0.75 mL/min. with 5 microliter injections. Integration of the UV trace was performed using area percent following a blank subtraction of the UV 280 nm signal from 4-13 minutes using ChemStations™ data processing software.

The separated nucleic acid-hydrophobic polymer conjugate will be concentrated using TFF and purity will be based on area percent using UV detection at 280 nm. This analytical method will determine the % conjugation of the nucleic acid-hydrophobic polymer conjugate as a fraction of the total amount of nucleic acid. Additionally, after removal of the solvents using TFF, the separated nucleic acid-hydrophobic polymer conjugate will be analyzed using mass spectrometry.

### Example 12

### Monitoring of Conjugation Reactions to Form oligonucleotide-C6-5050 PLGA Conjugate

### Materials

siRNA of several 20-21 base pair sequences was received from Thermo Fisher with a reduced hexyl thiol end group on the sense strand. Prior to the conjugation reaction, the siRNA was assessed for purity, e.g., degradation or dimerization of the siRNA, on an Agilent 1200 series automated HPLC equipped with a quaternary pump, diode array detector and autosampler with thermostatic control. An Agilent PLRP-S PS-DVB column 4000 Angstrom 4.6×150 mm was used and held at 25° C. The flow rate was 0.75 mL/min. The mobile phase used was a gradient solvent system using acetonitrile and water with tetra-n-butylammonium bromide (TBAB) as the ion pairing agent.

The siRNA was conjugated to the activated PLGA polymer, PLGA-SS-pyridine from Albany Molecular Research, Inc. (Albany N.Y.), on a 1:10 ratio of siRNA (2 mg/mL of siRNA in 95% DMSO/5% TE) to activated PLGA polymer (10× weight of activated polymer in 95% DMSO (ACS grade) 5% TE buffer) under inert conditions. Mobile Phase components DMF, ACN, and water were HPLC grade from Baker and anhydrous LiBr was ACS grade.

### Run Parameters

The nucleic acid-hydrophobic polymer conjugate run parameters were the same as the run parameters described in Example 11.

### Methods

The conjugation reaction was run at three different temperatures: room temperature; 40° C.; and 50° C. For the conjugation reactions run at room temperature and at 40° C., 5 microliter aliquots were removed from the reaction vials at 0 hours and again at 24 hours. The conjugation reaction run at 50° C. was first incubated at 60° C. for three hours prior to removing 5 microliter aliquots at 0 hours, 6 hours and 24 hours.

Monitoring of the conjugation reactions was performed by taking the aliquots (at times 0 hours, 6 hours, and 24 hours) and immediately injecting them onto an Agilent 1200 series automated HPLC equipped with a quaternary pump, diode array detector and autosampler with thermostatic control. A GL Sciences Inertsil® DIOL column 4.6×150 mm part #1290L150W046 was used and the column was held at 25° C. The flow rate was 0.75 mL/min. Integration of the UV trace was performed using area % following a blank subtraction of the UV 280 nm signal from 4-13 minutes using Agilent ChemStations™ data processing software.

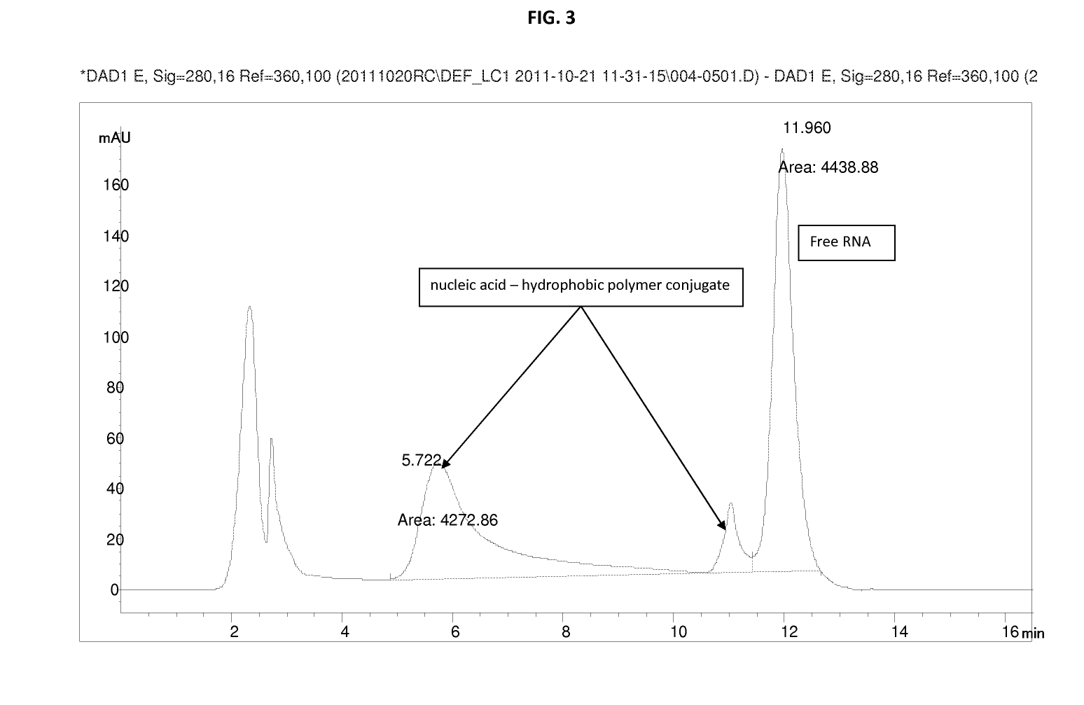

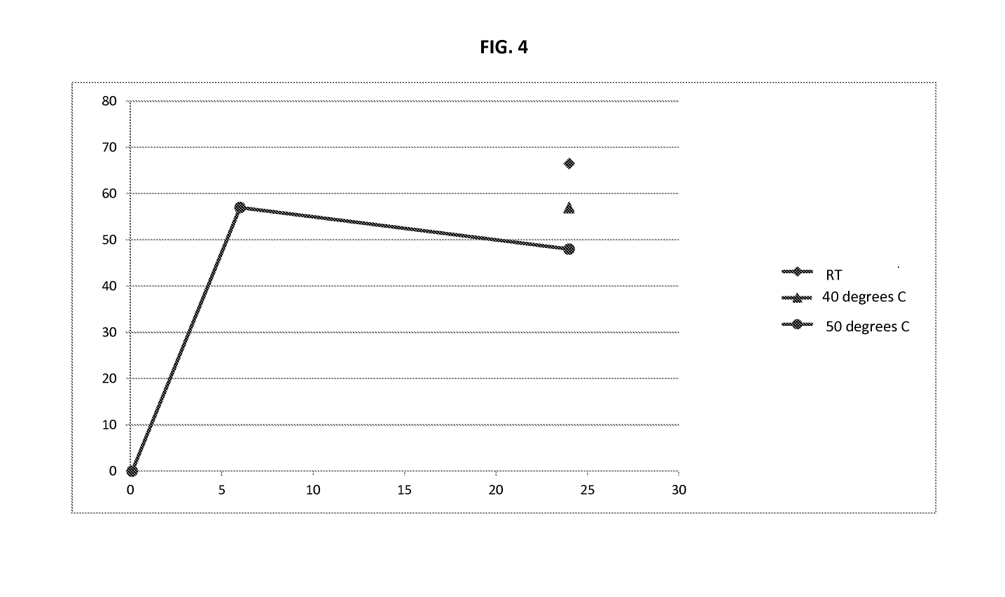

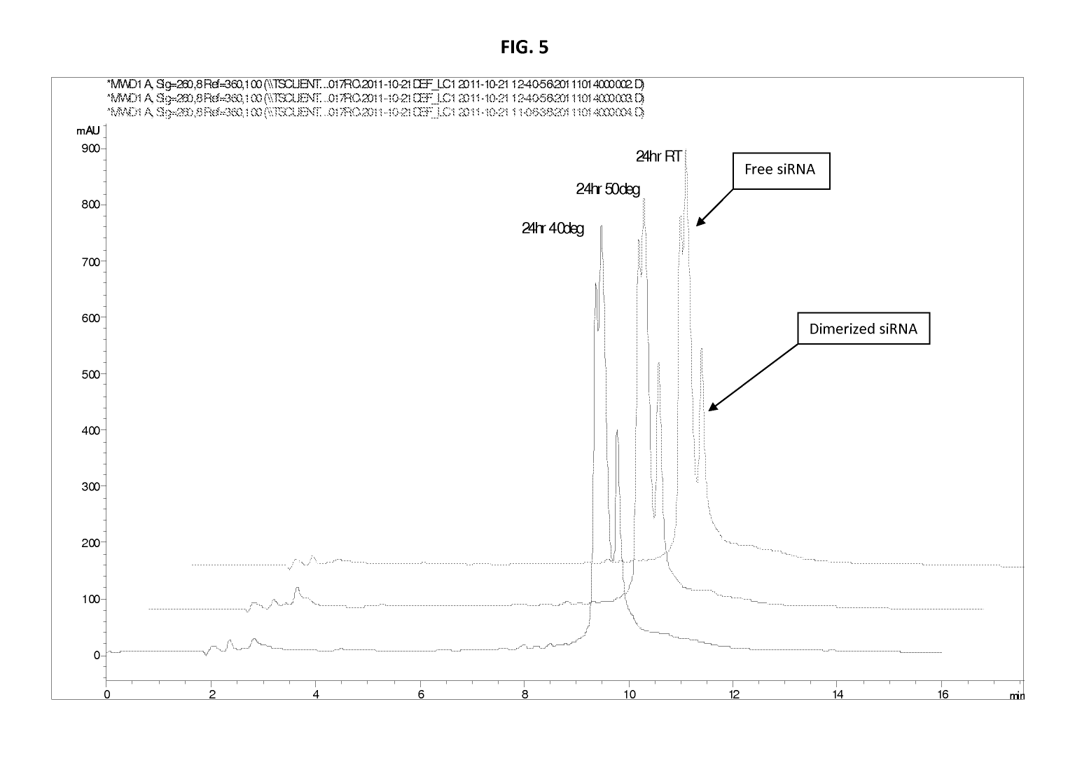

The conjugation reaction efficiency (measured as % conjugation) was determined by dividing the area % of the nucleic acid-hydrophobic polymer conjugate (integrating the peaks at 6 minutes and at 11 minutes) by the total area % (which included integrating the free RNA peak at 13 minutes). FIG. 3 shows the UV chromatogram for an aliquot taken at reaction time 24 hours for a conjugation reaction run at 40° C. FIG. 4 shows the conjugation efficiencies plotted as % conjugation as a function of time (hours) for the conjugation reactions run at room temperature (RT), 40° C., and 50° C. FIG. 4 shows that the % conjugation increased until about 5 hours, but then decreased at about 24 hours. An increase in temperature aided in the rate of the conjugation reaction. No significant change in the amount of siRNA degradation, e.g. siRNA dimerization, was observed at room temperature, 40° C. or 50° C. at 24 hours as shown in FIG. 5.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method of separating a nucleic acid-hydrophobic polymer conjugate from at least one other component, e.g., a contaminant, e.g., an unreacted starting material, of a mixture, the method comprising:
contacting the mixture with a first phase, e.g., a stationary phase, comprising a hydrogen bond donor or acceptor; and
applying a second phase, e.g., a mobile phase, comprising a polar solvent to selectively elute the nucleic acid-hydrophobic polymer conjugate from the stationary phase, thereby separating the nucleic acid-hydrophobic polymer conjugate from at least one other component of the mixture.

2. The method of claim 1, wherein the second phase, e.g., the mobile phase comprises a de-aggregating agent, e.g., an agent that disrupts hydrogen bonding of a nucleic acid molecule.

3. The method of claim 1, wherein the second phase, e.g., the mobile phase comprises a salt.

4. The method of claim 1, wherein the stationary phase is a normal phase.

5. The method of claim 1, wherein the hydrogen bond donor or acceptor is attached to a support, e.g., a solid support.

6. The method of claim 5, wherein the hydrogen bond donor or acceptor comprises hydroxyl moieties.

7-8. (canceled)

9. The method of claim 5, wherein the support has a diameter of 3 micrometers to 10 micrometers.

10. The method of claim 9, wherein the support comprises silica, e.g. porous silica, silica gel.

11. The method of claim 1, wherein the polar solvent comprises a polar aprotic solvent.

12. (canceled)

13. The method of claim 11, wherein the polar aprotic solvent is selected from acetonitrile, N,N-dimethylformamide, acetone, cyclohexanone, methyl ethyl ketone, methyl tert-butyl ether, diethyl ether, dimethyl ether, methyl acetate, ethyl acetate and nitromethane.

14. The method of claim 1, wherein the polar solvent comprises a polar protic solvent.

15. The method of claim 14, wherein the polar protic solvent is selected from an alcohol and water.

16-18. (canceled)

19. The method of claim 1, wherein the second phase, e.g., the mobile phase is applied as a gradient elution wherein the gradient elution increases in the % by volume of a first polar solvent and decreases in the % by volume of a second polar solvent, to elute the nucleic acid-hydrophobic polymer conjugate from the stationary phase.

20. The method of claim 19, wherein the % by volume of the first polar solvent increases, e.g., from 60% to at least 85%, from 60% to at least 90%, from 60% to at least 95%, or from 60% to at least 100%.

21. The method of claim 19, wherein the % by volume of the second polar solvent decreases from, e.g., 40% to 15% or less, 40% to 10% or less, 40% to 5% or less, or 40% to 0%.

22. The method of claim 19, wherein the first polar solvent comprises a solution of a salt, e.g., a lithium salt, and N,N-dimethylformamide.

23. The method of claim 19, wherein the second polar solvent comprises acetonitrile.

24-30. (canceled)

31. The method of claim 3, wherein the salt is an alkali metal halide, e.g., a lithium salt.

32. (canceled)

33. The method of claim 1, wherein the nucleic acid comprises RNA.

34. (canceled)

35. The method of claim 1, wherein the hydrophobic polymer comprises poly(lactic-co-glycolic acid) (PLGA).

36-49. (canceled)

50. A method of analyzing a mixture comprising a nucleic acid-hydrophobic polymer conjugate, e.g., a preparation made by the method of claim 1, for the presence of a component comprising: providing a mixture, e.g., a conjugation reaction mixture, comprising a nucleic acid or nucleic acid agent-hydrophobic polymer conjugate; and evaluating the mixture for the presence of one or more of an unconjugated nucleic acid, unconjugated hydrophobic polymer, or other conjugation reaction side product, thereby analyzing the mixture.

51. The method of claim 50, wherein said analyzing comprises applying spectrometric analysis.

52-60. (canceled)

61. The method of claim 1, wherein:
the first phase, e.g., the stationary phase comprises silica of a diameter of between 3 micrometers and 10 micrometers covalently bonded to hydroxypropyl moieties; and
the second phase, e.g., the mobile phase comprises acetonitrile, N,N-dimethylformamide, and lithium bromide.

62-71. (canceled)

72. A preparation of a nucleic acid-hydrophobic polymer conjugate prepared by the method of claim 1.

73-96. (canceled)
